(19) United States
(12) Patent Application Publication
(10) Pub. No.: US 2021/0154249 A1
(43) Pub. Date: May 27, 2021

(54) COXSACKIE VIRUS B FOR TREATING TUMORS

(71) Applicants: Xiamen University, Xiamen (CN); Yang Sheng Tang Company, Ltd., Hangzhou, Zhejiang (CN)

(72) Inventors: Tong CHENG, Xiamen (CN); Wei WANG, Xiamen (CN); Xiangzhong YE, Xiamen (CN); Wenkun FU, Xiamen (CN); Dequan PAN, Xiamen (CN); Jun ZHANG, Xiamen (CN); Ningshao XIA, Xiamen (CN)

(21) Appl. No.: 17/047,783

(22) PCT Filed: Apr. 15, 2019
(86) PCT No.: PCT/CN2019/082608

§ 371 Date: Oct. 15, 2020

(30) Foreign Application Priority Data
Apr. 16, 2018 (CN) 201810338046.2

(51) Int. Cl. A61K 35/76; C12N 7/00; A61P 35/04

(52) U.S. Cl. CPC A61K 35/76; A61P 35/04; C12N 7/00

(57) ABSTRACT

Provided are Coxsackie virus CVB1 or a modified form thereof, or a genomic sequence or cDNA sequence comprising CVB1 or the modified form thereof, or a nucleic acid molecule of a complement sequence of the genomic sequence or cDNA sequence, the use of same for treating tumors in subjects including humans, and the use of same in the preparation of a pharmaceutical composition for treating tumors in subjects including humans. Also provided is a method for treating tumors, comprising administering CVB1 or the modified form thereof, or the genomic sequence or cDNA sequence comprising CVB1 or the modified form thereof, or the nucleic acid molecule of the complement sequence of the genomic sequence or cDNA sequence to a subject in need thereof.

### TECHNICAL FIELD

The invention relates to the fields of virus and tumor therapy. In particular, the present invention relates to use of a CBV1 or modified form thereof, or of a nucleic acid molecule comprising a genomic nucleotide sequence of the CBV1 or modified form thereof or a complementary sequence thereof, for treating a tumor in a subject (e.g., a human), and for manufacture of a medicament for treating a tumor in a subject (e.g., a human). The present invention also relates to a method for treating a tumor, which comprises a step of administering to a subject in need thereof a CBV1 or modified form thereof, or a nucleic acid molecule comprising a genomic nucleotide sequence of the CBV1 or modified form thereof or a complementary sequence thereof.

### BACKGROUND ART

The current means for treatment of malignant tumors mainly include surgical treatment, chemotherapy and radiotherapy. These traditional therapies are not satisfactory in the treatment of metastasized tumors, and may further cause great harm to the health of patients. In contrast, as a new type of treatment, the method for treating tumors by using oncolytic viruses has the characteristics of high specificity, good effect, and low side effects, and thus is currently considered as a promising method for treating tumors.

An oncolytic virus is a virus that can self-replicate in tumor cells, thereby killing or lysing tumor cells, or arresting the growth of tumor cells. When used in in vivo treatment, oncolytic viruses exhibit specific selectivity for tumor cells and can directly induce the death of tumor cells, but have little or no effect on normal cells; meanwhile, oncolytic viruses can also stimulate the response of B lymphocytes and T lymphocytes in immune system, thereby indirectly killing tumor cells.

Enteroviruses with oncolytic activity that have been reported so far include the chimeric poliovirus for treatment of human solid tumors such as malignant gliomas (Dobrikova et al., Mol Ther 2008, 16(11): 1865-1872); echovirus ECHO1 that kills human gastric cancer cells and ovarian cancer cells (Shafren et al., Int J Cancer 2005, 115(2): 320-328; Haley et al., J Mol Med (Berl) 2009, 87(4): 385-399); and so on. However, it is still necessary to obtain a virus with both tumor specificity and tumor killing activity.

CVB1 belongs to the Enterovirus genus in the Picomaviridae family. Studies have found that CVB1 generally only causes mild symptoms such as fever, sneezing, and coughing in infected people after infection, but it may also cause severe chronic autoimmune diseases such as viral myocarditis, pancreatitis, hepatitis, aseptic meningitis, and insulin-dependent diabetes in neonates, infants and immunodeficiency adults (Rinehart et al., J Virol 1997, 71(5): 3986-3991). At present, there are no reports on oncolytic activity of CVB1 in the art.

### Contents of the Invention

In the present invention, unless otherwise stated, the scientific and technical terms used herein have the meaning commonly understood by those skilled in the art. In addition, the operating steps for cell culture, biochemistry, cell biology, nucleic acid chemistry and so on used herein are conventional steps widely used in the corresponding fields. Meanwhile, in order to better understand the present invention, definitions and explanations of related terms are provided below.

As used herein, the term “CVB1 (Coxsackivirus B1)” refers to one species of Coxsackivirus B of Enterovirus genus of Picomaviridae family, the genome of which is a single-stranded positive-sense RNA consisting of 5′ non-coding region (5′ UTR), one open reading frame (ORF), 3′ non-coding region (3′ UTR), and poly(A) tail. The ORF encoding a precursor polyprotein, which can be hydrolyzed and cleaved by its own protease to produce structural proteins VP1 to VP4 and non-structural proteins 2A, 2B, 2C, 3A, 3B, 3C, and 3D. In order to more clearly describe the present invention, the nucleic acid sequences corresponding to the above-described proteins in the CVB1 genome are called VP1 gene, VP2 gene, VP3 gene, VP4 gene, 2A gene, 2B gene, 2C gene, 3A gene, 3B gene, 3C gene and 3D gene. In the present invention, the expression “Coxsackivirus B1 (CVB1)” means wild-type CVB1, which can be isolated from sources in nature and has not been intentionally and artificially modified, examples of which include but are not limited to prototype strain Conn-5, and various clinical isolates (for example, the clinical isolates described in Example 1 of the present invention). The genomic sequence or cDNA sequence of wild-type CVB1 are well known in the art, and can be found in various public databases (for example, GenBank database accession number: MG780414).

As used herein, the term “modified form” of a virus refers to a modified virus obtained by modifying a wild-type virus, which retains the desired activities (e.g., oncolytic activity) of the wild-type virus. In the present invention, “modified form” of CVB1 includes, but is not limited to, a modified CVB1 virus, the genomic sequence of which has a substitution, insertion or deletion of one or more nucleotides compared to that of a wild-type CVB1, and at least retains the oncolytic activity of CVB1.

As used herein, the term “oncolytic virus” refers to a virus that can infect a tumor cell, replicate in the tumor cell, cause the death and lysis of the tumor cell, or prevent the growth of the tumor cell. Preferably, the virus has minimal toxic effects on a non-tumor cell.

As used herein, the term “tumor-specificity” refers to selectively exhibiting a biological function or activity in a tumor cell. For example, in the present invention, when the term “tumor specificity” is used to describe the killing selectivity of a virus, it means that the virus can selectively kill a tumor cell without killing or substantially not killing a non-tumor cell, or, the virus is more effective in killing a tumor cell than killing a non-tumor cell.

As used herein, the term “oncolytic activity” mainly comprises tumor-killing activity. When describing the oncolytic activity of a virus, the oncolytic activity of the virus can typically be measured by its ability to infect a tumor cell, its ability to replicate in the tumor cell, and/or its ability to kill the tumor cell. The oncolytic activity of a virus can be measured using any method known in the art. For example, the ability of a virus to infect a tumor cell can be evaluated by measuring the viral dose required to infect a given percentage of tumor cells (e.g., 50% of cells); the ability to replicate in a tumor cell can be evaluated by measuring the growth of the virus in the tumor cell; the ability to kill a tumor cell can be evaluated by monitoring cytopathic effect (CPE) or measuring tumor cell activity.

As used herein, the expression “cDNA sequence of CVB1” means that the DNA form of the viral genomic RNA sequence which differs from the RNA sequence only in that the ribonucleotides in the RNA sequence are replaced by corresponding deoxyribonucleotides, for example, uracil ribonucleotide (UMP) is replaced by thymine deoxyribonucleotide (dTMP).

As used herein, the term “exogenous nucleic acid” refers to an artificially introduced nucleotide sequence that is foreign to the original sequence. Exogenous nucleic acid includes, but is not limited to, any genes or nucleotide sequences not found in the viral genome. However, in the present invention, it is particularly preferred that the exogenous nucleic acid consists of at most 1500, for example at most 1200, at most 1000 nucleotides. In some cases, preferably, the exogenous nucleic acid encodes a protein or polypeptide having anti-tumor killing activity, such as a cytokine, or an anti-tumor protein or polypeptide; or, the exogenous nucleic acid includes a target sequence of microRNA (miRNA). In the present invention, the microRNA is preferably a microRNA having an expression level in tumor cells significantly lower than that in normal cells and/or having obvious tissue specificity, examples of which include, but are not limited to, miR-122, miR-192, miR-483, etc., which are specifically expressed in liver tissue; miR216a/b, miR217 and miR-375, which are specifically expressed in pancreatic tissue; miR-1, miR-133a/b, miR-208, etc., which are specifically expressed in heart; miR-192, miR-196a/b, miR-204, miR-215, etc., which are specifically expressed in kidney tissue; miR-133a/b, miR-206, etc., which are specifically expressed in muscle tissue; miR-124a, MiR-125a/b, miR-128a/b, miR-138, etc., which are specifically expressed in brain tissue; and miR-34, miR-122a, miR-26a, which are under-expressed in liver tumor tissue; miR-107, miR-96 and miR-196, which are under-expressed in pancreatic tumor tissue; miR-34, which is under-expressed in kidney tumor tissue; miR-143, miR-133a/b, which are under-expressed in bladder tumor tissue; miR-Let-7, miR-29, which are under-expressed in lung tumor tissue; and the like (see, for example, Ruiz A J and Russell S J. MicroRNAs and oncolytic viruses. [J]. Curr Opin Virol, 2015, 13: 40-48; all of which are incorporated herein by reference in their entireties).

In the present invention, when the modified CVB1 contains the target sequence of the above microRNA, it is regulated by the microRNA in the cells/tissues where the microRNA is highly expressed or specifically expressed, so that the replication of the oncolytic virus is weakened and even the killing activity is lost, while it can normally replicate in the tumor cells/tissues with low or no expression of the microRNA, and thus kill the tumor cells.

As used herein, the term “cytokine” has a meaning well known to those skilled in the art. However, in the present invention, when the oncolytic virus of the present invention is used to treat a tumor, it is particularly preferred that the cytokine is a cytokine that can be used for tumor treatment. Examples of “cytokine” include, but are not limited to, interleukins (e.g, IL-2, IL-12 and IL-15), interferons (e.g., IFNα, IFNβ, IFNγ), tumor necrosis factors (e.g., TNFα), colony stimulating factors (e.g., GM-CSF), and any combination thereof (see, for example, Ardolino M, Hsu J, Raulet D H. Cytokine treatment in cancer immunotherapy [J]. Oncotarget, 2015, 6 (23): 19346-19347).

As used herein, the term “anti-tumor protein or polypeptide” refers to a protein or polypeptide that has therapeutic activity against tumor, including but not limited to: (1) a protein or polypeptide that is toxic to a cell, or capable of inhibiting cell proliferation or inducing apoptosis, its examples include but are not limited to, thymidine kinase TK (TK/GCV), TRAIL, and FasL (see, for example, Candolfi M, King G D, Muhammad A G, et al. Evaluation of proapototic transgenes to use in combination with Flt3L in an immune-stimulatory gene therapy approach for Glioblastoma multiforme (GBM) [J]. FASEB J, 2008, 22: 1077.13); (2) a protein or polypeptide with immunotherapeutic effect, its examples include but are not limited to, single chain antibodies (scFv) against cytotoxic T lymphocyte-associated antigen 4 (anti-CTLA-4), programmed death receptor 1 (anti-PD-1) and programmed death ligand 1 (anti-PDL-1) (see, for example, Nolan E, Savas P, Policheni A N, et al. Combined immune checkpoint blockade as a therapeutic strategy for BRCA1-mutated breast cancer [J]. Science Trans Med, 2017, 9: eaa14922; all of which are incorporated herein by reference); (3) a protein or polypeptide that inhibits tumor angiogenesis, its examples include but are not limited to, single chain antibody (scFv) against vascular endothelial growth factor (anti-VEGF), VEGF-derived polypeptide (e.g., D(LPR), KSRVRKGKGQKRKRKKSRYK, etc.), and ATN-161 (see, for example, Rosca E V, Koskimaki J E, Rivera C G, et al. Anti-angiogenic peptides for cancer therapeutics [J]. Curr Pharm Biotechnol, 2011, 12 (8): 1101-1116; all of which are incorporated herein by reference).

As used herein, the term “scFv” refers to a single polypeptide chain comprising a heavy chain variable region (VH) and a light chain variable region (VL), where the VL and VH are ligated by a linker (See, for example, Bird et al., Science 242:423-426 (1988); Huston et al., Proc. Natl. Acad. Sci. USA 85:5879-5883 (1988); and Pluckthun, The Pharmacology of Monoclonal Antibodies, Volume 113, edited by Roseburg and Moore, Springer-Verlag, New York, pages 269-315 (1994)). Such scFv molecule can have a general structure: NH2-VL-linker-VH—COOH or NH2-VH-linker-VL-COOH.

As used herein, the term “identity” refers to the match degree between two proteins/polypeptides or between two nucleic acids. When two sequences for comparison have the same monomer sub-unit of base or amino acid at a certain site (e.g., each of two DNA molecules has an adenine at a certain site, or each of two proteins/polypeptides has a lysine at a certain site), the two molecules are identical at the site. The percent identity between two sequences is a function of the number of identical sites shared by the two sequences over the total number of sites for comparison×100. For example, if 6 of 10 sites of two sequences are matched, these two sequences have an identity of 60%. For example, DNA sequences: CTGACT and CAGGTT share an identity of 50% (3 of 6 sites are matched). Generally, the comparison of two sequences is conducted in a manner to produce maximum identity. Such alignment can be conducted by for example using a computer program such as Align program (DNAstar, Inc.) which is based on the method of Needleman, et al. (J. Mol. Biol. 48:443-453, 1970). The percentage of identity between two amino acid sequences can also be determined using the algorithm of E. Meyers and W. Miller (Comput. Appl. Biosci., 4:11-17 (1988)) which has been incorporated into the ALIGN program (version 2.0), using a PAM120 weight residue table, and with a gap length penalty of 12 and a gap penalty of 4. In addition, the percentage of identity between two amino acid sequences can be determined by the algorithm of Needleman and Wunsch (J. Mol. Biol. 48:444-453 (1970)) which has been incorporated into the GAP program in the GCG software package (available at http://www.gcg.com), using either a Blossum 62 matrix or a PAM250 matrix, and with a gap weight of 16, 14, 12, 10, 8, 6, or 4 and a length weight of 1, 2, 3, 4, 5, or 6.

As used herein, the term “vector” refers to a nucleic acid vehicle into which a polynucleotide can be inserted. When a vector enables expression of a protein encoded by an inserted polynucleotide, the vector is referred to as an expression vector. A vector can be introduced into a host cell by transformation, transduction, or transfection, so that the genetic material elements carried by the vector can be expressed in the host cell. The vector is well known to those skilled in the art and includes, but is not limited to: plasmids; phagemids; cosmids; artificial chromosomes, such as yeast artificial chromosomes (YAC), bacterial artificial chromosomes (BAC) or P1-derived artificial chromosomes (PAC); bacteriophages such as λ-phage or M13 phage and animal viruses. Animal viruses that can be used as vectors include, but are not limited to, retroviruses (including lentiviruses), adenoviruses, adeno-associated viruses, herpesviruses (such as herpes simplex virus), poxviruses, baculoviruses, papillomaviruses, and papovaviruses (such as SV40). A vector may contain a variety of elements that control expression, including, but not limited to, promoter sequences, transcription initiation sequences, enhancer sequences, elements for selection, and reporter genes. In addition, the vector may contain a replication initiation site.

As used herein, the term “internal ribosome entry site (IRES)” refers to a nucleotide sequence located in a messenger RNA (mRNA) sequence, which can initiate translation without relying on the 5′ cap structure. IRES is usually located in the 5′ untranslated region (5′UTR), but may also be located in other positions of the mRNA.

As used herein, the term “human rhinovirus 2 (HRV2)” refers to a virus in the family of picornaviridae whose genomic sequence or cDNA sequence is well known in the art, and can be found in various public databases (e.g., GenBank database accession number X02316.1).

As used herein, the expression “a nucleic acid molecule comprising a genomic sequence of CVB1 or a modified form thereof” or “a nucleic acid molecule comprises a genomic sequence of CVB1 or a modified form thereof” has the meaning commonly understood by those skilled in the art, that is, when the nucleic acid molecule is DNA, the nucleic acid molecule comprises a genomic sequence of CVB1 or a modified form thereof in form of DNA; when the nucleic acid molecule is RNA, the nucleic acid molecule comprises a genomic sequence of CVB1 or a modified form thereof.

As used herein, the term “pharmaceutically acceptable carrier and/or excipient” refers to a carrier and/or excipient that is pharmacologically and/or physiologically compatible with the subject and the active ingredient, which is well known in the art (see, for example, Remington's Pharmaceutical Sciences. Edited by Gennaro A R, 19th ed. Pennsylvania: Mack Publishing Company, 1995), and includes, but is not limited to: pH adjusting agents, surfactants, ionic strength enhancers, agents to maintain osmotic pressure, agents to delay absorption, diluents, adjuvants, preservatives, stabilizers, etc. For example, pH adjusting agents include, but are not limited to, phosphate buffered saline. Surfactants include, but are not limited to, cationic, anionic or non-ionic surfactants, such as Tween-80. Ionic strength enhancers include, but are not limited to, sodium chloride. Agents that maintain osmotic pressure include, but are not limited to, sugar, NaCl, and the like. Agents that delay absorption include, but are not limited to, monostearate and gelatin. Diluents include, but are not limited to, water, aqueous buffers (such as buffered saline), alcohols and polyols (such as glycerol), and the like. Adjuvants include, but are not limited to, aluminum adjuvants (such as aluminum hydroxide), Freund's adjuvants (such as complete Freund's adjuvant), and the like. Preservatives include, but are not limited to, various antibacterial and antifungal agents, such as thimerosal, 2-phenoxyethanol, parabens, trichloro-t-butanol, phenol, sorbic acid, and the like. Stabilizers have the meaning commonly understood by those skilled in the art, which can stabilize the desired activity (such as oncolytic activity) of the active ingredients in the drug, including but not limited to sodium glutamate, gelatin, SPGA, sugars (e.g., sorbitol, mannitol, starch, sucrose, lactose, dextran, or glucose), amino acids (e.g., glutamic acid, glycine), proteins (e.g., dried whey, albumin, or casein) or their degradation products (e.g., lactalbumin hydrolysates).

As used herein, the term “treating” refers to treating or curing a disease (e.g., a tumor), delaying the onset of symptoms of a disease (e.g., a tumor), and/or delaying the development of a disease (e.g., a tumor).

As used herein, the term “effective amount” refers to an amount that can effectively achieve the intended purpose. For example, a therapeutically effective amount can be an amount effective or sufficient to treat or cure a disease (e.g., a tumor), delay the onset of symptoms of a disease (e.g., a tumor), and/or delay the development of a disease (e.g., a tumor). Such an effective amount can be easily determined by a person skilled in the art or a doctor, and can be related to the intended purpose (such as treatment), the general health condition, age, gender, weight of the subject, severity of the disease to be treated, complications, administration routes, etc. The determination of such an effective amount is well within the capabilities of those skilled in the art.

As used herein, the term “subject” refers to a mammal, such as a primate mammal, such as a human. In certain embodiments, the subject (e.g., a human) has a tumor, or is at risk for having a tumor.

After a lot of experiments and repeated explorations, the inventors of the present application have unexpectedly discovered that CVB1 has a broad-spectrum and significant tumor cell killing ability. Based on this discovery, the inventors have developed a new oncolytic virus for treating a tumor and a method for tumor treatment based on the virus.

Therefore, in a first aspect, the present invention provides use of a Coxsackievirus B1 (CVB1) or a modified form thereof or a nucleic acid molecule for treating a tumor in a subject, or for manufacture of a medicine for treating a tumor in a subject; wherein the nucleic acid molecule comprises a sequence selected from the following:

(1) a genomic sequence or cDNA sequence of CVB1 or modified form thereof; and

(2) a complementary sequence of the genomic sequence or cDNA sequence.

In certain preferred embodiments, the CVB1 is a wild-type CVB1. In certain preferred embodiments, the CVB1 may be a clinical isolate isolated from an individual infected with Coxsackievirus B1.

In certain preferred embodiments, the genomic sequence of CVB1 or modified form thereof has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% to the nucleotide sequence as shown in SEQ ID NO:12. In certain preferred embodiments, the genomic sequence of CVB1 or modified form thereof is the nucleotide sequence as shown in SEQ ID NO:12.

In certain preferred embodiments, the cDNA sequence of CVB1 or modified form thereof has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% to the nucleotide sequence as shown in SEQ ID NO:1. In certain preferred embodiments, the cDNA sequence of CVB1 or modified form thereof is the nucleotide sequence as shown in SEQ ID NO: 1.

In certain preferred embodiments, the modified form is a modified CVB1 that has a substitution, insertion or deletion of one or more nucleotides in the genome compared to a wild-type CVB1.

In certain preferred embodiments, as compared to the wild-type CVB1, the modified CVB1 has one or more modifications selected from the following:

(1) one or more mutations in an untranslated region (e.g. 5′UTR or 3′UTR);

(2) an insertion of one or more exogenous nucleic acids;

(3) a deletion or mutation of one or more endogenous genes; and

(4) any combination of the above three items.

In certain preferred embodiments, the modified CVB1 comprises one or more mutations in a 5′ untranslated region (5′UTR).

In certain preferred embodiments, the modified CVB1 has a substitution of all or part of the 5′UTR sequence. In certain preferred embodiments, the internal ribosome entry site (IRES) sequence in the 5′UTR of the modified CVB1 is replaced with an exogenous IRES sequence, such as an internal ribosome entry site sequence of human rhinovirus 2 (HRV2). In certain preferred embodiments, the internal ribosome entry site sequence of human rhinovirus 2 (HRV2) is shown in SEQ ID NO:2.

The use of the internal ribosome entry site sequence of human rhinovirus 2 (HRV2) is advantageous in some cases, for example, to improve the tumor specificity of oncolytic viruses. It has been previously reported that in normal human nerve cells, the internal ribosome entry site sequence of human rhinovirus 2 is specifically bound by host RNA-binding proteins (DRBP76 and NF45), thereby preventing the recruitment of factors such as elF4G (Merrill et al. J Virol 2006,80 (7): 3147-3156; Merrill and Gromeier, J Virol 2006,80 (14): 6936-6942; Neplioueva et al. PLoS One 2010,5(7): e11710); in the meantime, without the support of Raf/Erk1/2/MAPK and other signaling pathways, ribosomes can hardly be bound to the internal ribosome entry site sequence of human rhinovirus 2 and therefore translation of viral protein cannot be initiated (Dobrikov et al., Mol Cell Biol 2011, 31 (14): 2947-2959; Dobrikov et al., Mol Cell Biol 2013, 33 (5): 937-946). In human glioma tumor cells, the internal ribosome entry site of human rhinovirus 2 is not affected by the above two factors, and thus can normally initiate transcription and translation of viral protein. Therefore, in some cases, replacing the internal ribosome entry site sequence of CVB1 with the internal ribosome entry site sequence of human rhinovirus 2 is beneficial to avoid or reduce the toxic and side effects of the virus of the present invention on normal human nerve cells without affecting the use of the virus in the treatment of human gliomas.

In certain preferred embodiments, the modified CVB1 comprises an exogenous nucleic acid.

In certain preferred embodiments, the exogenous nucleic acid encodes a cytokine (e.g., GM-CSF, preferably human GM-CSF), or an anti-tumor protein or polypeptide (e.g., scFv against PD-1 or PD-L1, preferably, scFv against human PD-1 or PD-L1). In certain preferred embodiments, the exogenous nucleic acid is inserted between 5′UTR and VP4 gene, or between VP1 gene and 2A gene of a genome of the modified CVB1.

In certain preferred embodiments, the exogenous nucleic acid comprises a target sequence of microRNA (miRNA) (e.g., miR-133 or miR-206). In certain preferred embodiments, the target sequence of microRNA is inserted in a 3′ untranslated region (3′UTR) of a genome of the modified CVB1.

It has been previously reported that the expression level of certain microRNAs in tumor cells is significantly lower than that in normal cells and/or has obvious tissue specificity. Thus, in some cases, it is advantageous that the modified CVB1 of the present invention comprises a target sequence of such microRNAs, because such microRNAs that are highly expressed in normal cells or tissues can reduce or even block the replication of the modified CVB1 in the normal cells or tissues via the corresponding target sequence, thereby reducing or even avoiding the toxic and side effects of the modified CVB1 on non-tumor cells. Such microRNAs include, but are not limited to, miR-133, miR-206, miR-1, miR-143, miR-145, miR-217, let-7, miR-15, miR-16, etc. (see, for example, PCT International Application WO2008103755A1, US patent application US20160143969A1, or Baohong Zhang et al., Developmental Biology, Volume 302, Issue 1, 1 Feb. 2007, Pages 1-12; all of which are incorporated herein in their entirety by reference).

In certain preferred embodiments, the exogenous nucleic acid includes a target sequence of one or more (e.g., 2, 3 or 4) microRNAs as described above. In certain preferred embodiments, the exogenous nucleic acid comprises a target sequence of miR-133 and/or miR-206. In certain preferred embodiments, the target sequence of miR-133 is shown in SEQ ID NO:3. In certain preferred embodiments, the target sequence of miR-206 is shown in SEQ ID NO:4. In some cases, the insertion of the target sequence of miR-133 and/or miR-206 is advantageous. This is because miR-133 and miR-206 are specifically expressed in muscle tissue, so that the insertion of the target sequence of miR-133 and/or miR-206 into the modified CVB1 may change the tissue tropism of the oncolytic virus, thereby reducing or avoiding damage to normal muscle tissue.

In certain preferred embodiments, the modified CVB1 comprises at least one insertion of the exogenous nucleic acid as described above and/or at least one mutation in the untranslated region as described above.

In certain preferred embodiments, the genomic sequence of the modified CVB1 has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% to a nucleotide sequence selected from the following: nucleotide sequences as shown in SEQ ID NOs: 13-16. In certain preferred embodiments, the genomic sequence of the modified CVB1 is any one selected from the nucleotide sequences as shown in SEQ ID NOs: 13-16.

In certain preferred embodiments, the cDNA sequence of the modified CVB1 has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% to a nucleotide sequence selected from the following: nucleotide sequences as shown in SEQ ID NOs: 8-11. In certain preferred embodiments, the cDNA sequence of the modified CVB1 is any one selected from the nucleotide sequences as shown in SEQ ID NOs: 8-11.

The modified CVB1 of the present invention can be obtained by reverse genetics technology, which is known in the art, for example, see Yang L S, Li S X, Liu Y J, et al. Virus Res, 2015, 210: 165-168; Hou W H, Yang L S, Li S X, et al. Virus Res, 2015, 205: 41-44; all of which are incorporated herein by reference. In such embodiments, the cDNA of wild-type CVB1 is typically subjected to modification (e.g., insertion of an exogenous nucleic acid, deletion or mutation of an endogenous gene, or mutation in an untranslated region) to obtain the modified CVB1.

The CVB1 or modified form thereof according to the present invention may be subjected to a pretreatment to reduce or eliminate an immune response against the virus in a subject, wherein the pretreatment may comprise: packaging the CVB1 in a liposome or micelle, and/or using a protease (e.g., chymotrypsin or trypsin) to remove a capsid protein of the virus to reduce a humoral and/or cellular immunity against the virus in the host.

In the present invention, the CVB1 or modified form thereof as described herein can be serially passaged for adaptation in tumor cells. In certain preferred embodiments, the tumor cells may be tumor cell lines or tumor cell strains known in the art, or tumor cells obtained by in vivo surgical resection or clinical isolation from an individual (e.g., a subject) having a tumor. In certain preferred embodiments, the CVB1 or modified form thereof is serially passaged for adaptation in tumor cells obtained from an individual (e.g., a subject) having a tumor. In certain preferred embodiments, the tumor cells are obtained by surgical resection or clinical isolation from an individual (e.g., a subject) having a tumor. In certain preferred embodiments, the method of serial passaging for adaptation comprises a plurality of (e.g., at least 5, at least 10, at least 15, at least 20) cycles that consists of the following processes: 1) infecting a target tumor cell with the virus; 2) harvesting the virus in the supernatant; and 3) reinfecting a fresh target tumor cell with the obtained virus.

In certain preferred embodiments, the CVB1 and modified form thereof as described above may be used in combination. Therefore, the medicament may comprise one or several of the CVB1 and modified forms thereof.

In certain preferred embodiments, the nucleic acid molecule consists of a genomic sequence or cDNA sequence of the CVB1 or modified form thereof as described herein, or a complementary sequence of the genomic sequence or cDNA sequence. In certain preferred embodiments, the nucleic acid molecule has a genomic sequence of the CVB1 or modified form thereof as described herein. In certain preferred embodiments, the nucleic acid molecule is RNA. In certain preferred embodiments, the nucleic acid molecule has a nucleotide sequence as shown in any one of SEQ ID NOs: 12-16.

In certain preferred embodiments, the nucleic acid molecule is a vector (e.g., cloning vector or expression vector) comprising a genomic sequence or cDNA sequence of the CVB1 or modified form thereof as described herein, or a complementary sequence of the genomic sequence or cDNA sequence. In certain preferred embodiments, the nucleic acid molecule is a vector (e.g., cloning vector or expression vector) comprising a cDNA sequence of the CVB1 or modified form thereof as described herein, or a complementary sequence of the cDNA sequence.

In certain preferred embodiments, the nucleic acid molecule comprises a complementary sequence of the genomic sequence of the CVB1 or modified form thereof. In certain preferred embodiments, the complementary sequence is complementary to a nucleotide sequence selected from the following:

(1) a nucleotide sequence as shown in SEQ ID NO: 12;

(2) a nucleotide sequence having a sequence identity of at least 70%, at least 80%, at least 85%, at least 90%, at least 91%, at least 92%, at least 93%, at least 94%, at least 95%, at least 96%, at least 97%, at least 98%, at least 99%, or 100% to the nucleotide sequence as shown in SEQ ID NO:12;

(3) a nucleotide sequence as shown in any one of SEQ ID NOs: 13-16; and

(4) a nucleotide sequence having a sequence identity of at least 70%, at least 80%, at least 85%, at least 90%, at least 91%, at least 92%, at least 93%, at least 94%, at least 95%, at least 96%, at least 97%, at least 98%, at least 99%, or 100% to the nucleotide sequence as shown in any one of SEQ ID NOs: 13-16.

In certain preferred embodiments, the nucleic acid molecule comprises a complementary sequence of the cDNA sequence of the CVB1 or modified form thereof. In certain preferred embodiments, the complementary sequence is complementary to a nucleotide sequence selected from the following:

(1) a nucleotide sequence as shown in SEQ ID NO: 1;

(2) a nucleotide sequence having a sequence identity of at least 70%, at least 80%, at least 85%, at least 90%, at least 91%, at least 92%, at least 93%, at least 94%, at least 95%, at least 96%, at least 97%, at least 98%, at least 99%, or 100% to the nucleotide sequence as shown in SEQ ID NO:1;

(3) a nucleotide sequence as shown in any one of SEQ ID NOs: 8-11; and

(4) a nucleotide sequence having a sequence identity of at least 70%, at least 80%, at least 85%, at least 90%, at least 91%, at least 92%, at least 93%, at least 94%, at least 95%, at least 96%, at least 97%, at least 98%, at least 99%, or 100% to the nucleotide sequence as shown in any one of SEQ ID NOs: 8-11.

The nucleic acid molecule of the present invention can be delivered by any means known in the art, for example, a naked nucleic acid molecule (e.g., a naked RNA) can be directly injected, or a non-viral delivery system can be used. The non-viral delivery system can be obtained from a variety of materials well known in the art, including, but not limited to, the materials described in detail in “Yin H, et al. Nat Rev Genet. 2014 August; 15(8): 541-55.” and “Riley M K, Vermerris W. Nanomaterials (Basel). 2017 Apr. 28; 7(5). Pii: E94.”, which are incorporated herein by reference in their entirety, such as liposomes, inorganic nanoparticles (such as gold nanoparticles), polymers (such as PEG).

In certain preferred embodiments, the medicament comprises a therapeutically effective amount of the CVB1 and/or modified form thereof, or a therapeutically effective amount of the nucleic acid molecule as described herein. In certain preferred embodiments, the medicament may be in any form known in the medical arts. For example, the medicament may be in the form of a tablet, a pill, a suspension, an emulsion, a solution, a gel, a capsule, a powder, a granule, an elixir, a lozenge, a suppository, or an injection (including injection liquid, lyophilized powder) and so on. In some embodiments, the medicament is an injection liquid or a lyophilized powder.

In certain preferred embodiments, the medicament further comprises a pharmaceutically acceptable carrier or excipient. In certain preferred embodiments, the medicament comprises a stabilizer.

In certain preferred embodiments, the medicament optionally further comprises an additional pharmaceutically active agent. In certain preferred embodiments, the CVB1 or modified form thereof according to the present invention, or the nucleic acid molecule of the present invention, is used in combination with an additional pharmaceutically active agent. In a preferred embodiment, the additional pharmaceutically active agent is a drug with anti-tumor activity, such as an additional oncolytic virus, chemotherapeutic agent or immunotherapeutic agent.

In the present invention, the additional oncolytic virus includes but is not limited to herpes virus, adenovirus, parvovirus, reovirus, Newcastle disease virus, vesicular stomatitis virus, measles virus, or any combination thereof. The chemotherapeutic agent includes but is not limited to 5-fluorouracil, mitomycin, methotrexate, hydroxyurea, cyclophosphamide, dacarbazine, mitoxantrone, anthracyclines (e.g., epirubicin or doxorubicin), etoposide, platinum compounds (e.g., carboplatin or cisplatin), taxanes (e.g., paclitaxel or docetaxel), or any combination thereof. The immunotherapeutic agent includes but is not limited to immune checkpoint inhibitor (e.g., PD-L1/PD-1 inhibitor or CTLA-4 inhibitor), tumor-specific targeting antibody (e.g., rituximab or herceptin), or any combination thereof.

In certain preferred embodiments, the medicament comprises a unit dose of the CVB1 and/or modified form thereof, for example, comprises at least 1×10² pfu, at least 1×10³ pfu, at least 1×10⁴ pfu, 1×10⁵ pfu, 1×10⁶ pfu, at least 1×10⁷ pfu, at least 1×10⁸ pfu, at least 1×10⁹ pfu, at least 1×10¹⁰ pfu, at least 1×10¹¹ pfu, at least 1×10¹² pfu, at least 1×10¹³ pfu, at least 1×10¹⁴ pfu or at least 1×10¹⁶ pfu of the CVB1 and/or modified form thereof. In certain preferred embodiments, the medicament comprises 1×10² pfu to 1×10¹⁷ pfu of the CVB1 and/or modified form thereof.

In certain preferred embodiments, the medicament comprises a unit dose of the nucleic acid molecule as described herein, for example, comprises 3×10¹⁰ to 3×10¹⁴ virus genome copies of the nucleic acid molecule.

In certain preferred embodiments, the medicament can be administered in combination with an additional therapy. This additional therapy may be any therapy known for tumors, such as surgery, chemotherapy, radiation therapy, immunotherapy, hormone therapy, or gene therapy. This additional therapy can be administered before, at the same time, or after administration of the medicament.

In certain preferred embodiments, the tumor is selected from colorectal cancer, gastric cancer, lung cancer, liver cancer, ovarian cancer, endometrial cancer, cervical cancer, melanoma, breast cancer, kidney cancer, pancreatic cancer, lymphoma, osteogenic sarcoma, prostate cancer, glioma, neuroblastoma, tongue cancer, nasopharyngeal cancer, squamous cell carcinoma of nasal septum, pharyngeal squamous cell carcinoma, squamous cell carcinoma of submandibular gland, laryngeal caner, thyroid cancer, thyroid ductal carcinoma and bladder cancer. In certain preferred embodiments, the tumor is selected from lung cancer, esophageal cancer, ovarian cancer, endometrial cancer, pancreatic cancer, tongue cancer, kidney cancer, prostate cancer, nasopharyngeal cancer, and bladder cancer.

In certain preferred embodiments, the subject is a mammal, such as a human.

In a second aspect, the present invention provides a method for treating a tumor, which comprises a step of administering to a subject in need thereof an effective amount of a CBV1 or modified form thereof, or an effective amount of a nucleic acid molecule; wherein, the nucleic acid molecule comprises a sequence selected from the following:

(1) a genomic sequence or cDNA sequence of the CBV1 or modified form thereof; and

(2) a complementary sequence of the genomic sequence or cDNA sequence.

In certain preferred embodiments, the subject is administered with the CBV1. In certain preferred embodiments, the CBV1 is a wild-type CBV1. In certain preferred embodiments, the CBV1 may be a clinical isolate isolated from an individual infected with a Coxsackievirus B1.

In certain preferred embodiments, the genomic sequence of the CBV1 or modified form thereof has a sequence identity of at least 70%, at least 80%, at least 85%, at least 90%, at least 91%, at least 92%, at least 93%, at least 94%, at least 95%, at least 96%, at least 97%, at least 98%, at least 99%, or 100%, to the nucleotide sequence as shown in SEQ ID NO: 12. In certain preferred embodiments, the genomic sequence of the CBV1 or modified form thereof is the nucleotide sequence as shown in SEQ ID NO: 12.

In certain preferred embodiments, the cDNA sequence of the CBV1 or modified form thereof has a sequence identity of at least 70%, at least 80%, at least 85%, at least 90%, at least 91%, at least 92%, at least 93%, at least 94%, at least 95%, at least 96%, at least 97%, at least 98%, at least 99%, or 100%, to the nucleotide sequence as shown in SEQ ID NO: 1. In certain preferred embodiments, the cDNA sequence of the CBV1 or modified form thereof is the nucleotide sequence as shown in SEQ ID NO: 1.

In certain preferred embodiments, the subject is administered with the modified form of the CBV1. In certain preferred embodiments, the modified form is a modified CBV1, which has a substitution, insertion or deletion of one or more nucleotides in the genome as compared to a wild-type CBV1.

In certain preferred embodiments, the modified CBV1 has one or more modifications selected from the following as compared to a wild-type CBV1:

(1) one or more mutations in an untranslated region (e.g. 5′UTR or 3′UTR);

(2) an insertion of one or more exogenous nucleic acids;

(3) a deletion or mutation of one or more endogenous genes; and

(4) any combination of the above three items.

In certain preferred embodiments, the modified CVB1 comprises one or more mutations in a 5′ untranslated region (5′UTR).

In certain preferred embodiments, the modified CVB1 has a substitution of all or part of the 5′UTR sequence. In certain preferred embodiments, the internal ribosome entry site (IRES) sequence in the 5′UTR of the modified CVB1 is replaced with an exogenous IRES sequence, such as an internal ribosome entry site sequence of human rhinovirus 2 (HRV2). In certain preferred embodiments, the internal ribosome entry site sequence of human rhinovirus 2 (HRV2) is shown in SEQ ID NO:2.

In certain preferred embodiments, the modified CVB1 comprises an exogenous nucleic acid.

In certain preferred embodiments, the exogenous nucleic acid encodes a cytokine (e.g., GM-CSF, preferably human GM-CSF), or an anti-tumor protein or polypeptide (e.g., scFv against PD-1 or PD-L1, preferably, scFv against human PD-1 or PD-L1). In certain preferred embodiments, the exogenous nucleic acid is inserted between 5′UTR and VP4 gene, or between VP1 gene and 2A gene of a genome of the modified CVB1.

In certain preferred embodiments, the exogenous nucleic acid comprises a target sequence of microRNA (miRNA) (e.g., miR-133 or miR-206). In certain preferred embodiments, the target sequence of microRNA is inserted in a 3′ untranslated region (3′UTR) of a genome of the modified CVB1.

In certain preferred embodiments, the exogenous nucleic acid includes a target sequence of one or more (e.g., 2, 3 or 4) microRNAs as described above. In certain preferred embodiments, the exogenous nucleic acid comprises the target sequence of miR-133 and/or miR-206. In certain preferred embodiments, the target sequence of miR-133 is shown in SEQ ID NO:3. In certain preferred embodiments, the target sequence of miR-206 is shown in SEQ ID NO:4.

In certain preferred embodiments, the modified CVB1 comprises at least one insertion of the exogenous nucleic acid as described above and/or at least one mutation in the untranslated region as described above.

In certain preferred embodiments, the genomic sequence of the modified CVB1 has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% to a nucleotide sequence selected from the following: nucleotide sequences as shown in SEQ ID NOs: 13-16. In certain preferred embodiments, the genomic sequence of the modified CVB1 is any one selected from the nucleotide sequences as shown in SEQ ID NOs: 13-16.

In certain preferred embodiments, the cDNA sequence of the modified CVB1 has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% to a nucleotide sequence selected from the following: nucleotide sequences as shown in SEQ ID NOs: 8-11. In certain preferred embodiments, the cDNA sequence of the modified CVB1 is any one selected from the nucleotide sequences as shown in SEQ ID NOs: 8-11.

In certain preferred embodiments, the CVB1 and modified forms thereof as described above may be used in combination. Therefore, one or more of the CVB1 and modified forms thereof can be administered to the subject.

In certain preferred embodiments, the nucleic acid molecule as described herein is administered to the subject.

In certain preferred embodiments, the nucleic acid molecule consists of a genomic sequence or cDNA sequence of the CVB1 or modified form thereof as described herein, or a complementary sequence of the genomic sequence or cDNA sequence. In certain preferred embodiments, the nucleic acid molecule has a genomic sequence of the CVB1 or modified form thereof as described herein. In certain preferred embodiments, the nucleic acid molecule is RNA. In certain preferred embodiments, the nucleic acid molecule has a nucleotide sequence as shown in any one of SEQ ID NOs: 12-16.

In certain preferred embodiments, the nucleic acid molecule is a vector (e.g., cloning vector or expression vector) comprising a genomic sequence or cDNA sequence of the CVB1 or modified form thereof as described herein, or a complementary sequence of the genomic sequence or cDNA sequence. In certain preferred embodiments, the nucleic acid molecule is a vector (e.g., cloning vector or expression vector) comprising a cDNA sequence of the CVB1 or modified form thereof as described herein, or a complementary sequence of the cDNA sequence.

In certain preferred embodiments, the nucleic acid molecule comprises a complementary sequence of the genomic sequence of the CVB1 or modified form thereof. In certain preferred embodiments, the complementary sequence is complementary to a nucleotide sequence selected from the following:

(1) a nucleotide sequence as shown in SEQ ID NO: 12;

(2) a nucleotide sequence having a sequence identity of at least 70%, at least 80%, at least 85%, at least 90%, at least 91%, at least 92%, at least 93%, at least 94%, at least 95%, at least 96%, at least 97%, at least 98%, at least 99%, or 100% to the nucleotide sequence as shown in SEQ ID NO:12;

(3) a nucleotide sequence as shown in any one of SEQ ID NOs: 13-16; and

(4) a nucleotide sequence having a sequence identity of at least 70%, at least 80%, at least 85%, at least 90%, at least 91%, at least 92%, at least 93%, at least 94%, at least 95%, at least 96%, at least 97%, at least 98%, at least 99%, or 100% to the nucleotide sequence as shown in any one of SEQ ID NOs: 13-16.

In certain preferred embodiments, the nucleic acid molecule comprises a complementary sequence of the cDNA sequence of the CVB1 or modified form thereof. In certain preferred embodiments, the complementary sequence is complementary to a nucleotide sequence selected from the following:

(1) a nucleotide sequence as shown in SEQ ID NO: 1;

(2) a nucleotide sequence having a sequence identity of at least 70%, at least 80%, at least 85%, at least 90%, at least 91%, at least 92%, at least 93%, at least 94%, at least 95%, at least 96%, at least 97%, at least 98%, at least 99%, or 100% to the nucleotide sequence as shown in SEQ ID NO:1;

(3) a nucleotide sequence as shown in any one of SEQ ID NOs: 8-11; and

(4) a nucleotide sequence having a sequence identity of at least 70%, at least 80%, at least 85%, at least 90%, at least 91%, at least 92%, at least 93%, at least 94%, at least 95%, at least 96%, at least 97%, at least 98%, at least 99%, or 100% to the nucleotide sequence as shown in any one of SEQ ID NOs: 8-11.

In the present invention, the nucleic acid molecule of the present invention can be delivered by any means known in the art, for example, a naked nucleic acid molecule (e.g., naked RNA) can be directly injected, or a non-viral delivery system can be used. The non-viral delivery system can be obtained from a variety of materials well known in the art, including, but not limited to, the materials described in detail in “Yin H, et al. Nat Rev Genet. 2014 August; 15(8): 541-55.” and “Riley M K, Vermerris W. Nanomaterials (Basel). 2017 Apr. 28; 7(5). Pii: E94.”, which are incorporated herein by reference in their entirety, such as liposomes, inorganic nanoparticles (such as gold nanoparticles), polymers (such as PEG).

In certain preferred embodiments, the CVB1 and/or modified form thereof, or nucleic acid molecules as described herein, can be formulated and administered as a pharmaceutical composition. Such pharmaceutical composition may comprise a therapeutically effective amount of the CVB1 and/or modified form thereof, or a therapeutically effective amount of the nucleic acid molecule as described herein. In certain preferred embodiments, the pharmaceutical composition may be in any form known in the medical arts. For example, the pharmaceutical composition may be a tablet, pill, suspension, emulsion, solution, gel, capsule, powder, granule, elixir, lozenge, suppository, injection (including injection liquid, lyophilized powder), and other forms. In some embodiments, the pharmaceutical composition is an injection liquid or a lyophilized powder.

In certain preferred embodiments, the pharmaceutical composition further comprises a pharmaceutically acceptable carrier or excipient. In certain preferred embodiments, the pharmaceutical composition comprises a stabilizer.

In the present invention, the CVB1 and/or modified forms thereof, or the nucleic acid molecules as described herein can be administered to a subject by various suitable routes. In some cases, the administration route of the CVB1 and/or modified form thereof, or the nucleic acid molecules as described herein, depends on the location and type of tumor. For example, for a solid tumor that is easily accessible, the virus or nucleic acid molecule is optionally administered by injection directly into the tumor (e.g., intratumoral injection); for a tumor of hematopoietic system, the virus or nucleic acid molecule can be administered by intravenous or other intravascular routes; for a tumor that is not easily accessible in the body (e.g., metastases), the virus or nucleic acid molecule can be administered systematically so that it can run over the whole body and thereby reaching the tumor (e.g., intravenous or intramuscular injection). Optionally, the virus or nucleic acid molecule of the present invention can be administrated via subcutaneous, intraperitoneal, intrathecal (e.g., for brain tumors), topical (e.g., for melanoma), oral (e.g., for oral or esophageal cancer), intranasal or inhalation spray (e.g., for lung cancer) routes, and the like. In certain preferred embodiments, the CVB1 and/or modified form thereof of the invention, or the nucleic acid as described herein, can be administered via intradermal, subcutaneous, intramuscular, intravenous and oral routes, and the like.

In certain preferred embodiments, the method further comprises administering an additional pharmaceutically active agent having anti-tumor activity. Such additional pharmaceutically active agent may be administered before, simultaneously or after administration of the CVB1 and/or modified form thereof, or the nucleic acid molecule as described herein.

In certain preferred embodiments, the additional pharmaceutically active agent comprises additional oncolytic virus, chemotherapeutic agent, or immunotherapeutic agent.

In the present invention, the additional oncolytic virus includes but is not limited to herpes virus, adenovirus, parvovirus, reovirus, Newcastle disease virus, vesicular stomatitis virus, measles virus, or any combination thereof. The chemotherapeutic agent includes but is not limited to 5-fluorouracil, mitomycin, methotrexate, hydroxyurea, cyclophosphamide, dacarbazine, mitoxantrone, anthracyclines (e.g., epirubicin or doxorubicin), etoposide, platinum compounds (e.g., carboplatin or cisplatin), taxanes (e.g., paclitaxel or docetaxel), or any combination thereof. The immunotherapeutic agent includes but is not limited to immune checkpoint inhibitor (e.g., PD-L1/PD-1 inhibitor or CTLA-4 inhibitor), tumor-specific targeting antibody (e.g., rituximab or herceptin), or any combination thereof.

In certain preferred embodiments, the CVB1 and/or modified form thereof can be administered in any amount from 1 to 1×10¹⁵ pfu/kg of the subject's body weight, for example, the CVB1 and/or modified form thereof can be administered in an amount of at least 1×10³ pfu/kg, at least 1×10⁴ pfu/kg, 1×10⁵ pfu/kg, 1×10⁶ pfu/kg, at least 1×10⁷ pfu/kg, at least 1×10⁸ pfu/kg, at least 1×10⁹ pfu/kg, at least 1×10¹⁰ pfu/kg, at least 1 ×10¹¹ pfu/kg, or at least 1×10¹² pfu/kg of the subject's body weight. In certain preferred embodiments, the nucleic acid molecule as described herein can be administered in any amount from 3×10¹⁰ to 3×10¹⁴ virus genome copies per kg of the subject's body weight. In certain preferred embodiments, the CVB1 and/or modified form thereof or the nucleic acid molecule as described herein can be administered 3 times per day, 2 times per day, once per day, once every two days, or once per week, and the above-mentioned dosage regimen may be optionally repeated weekly or monthly as appropriate.

In certain preferred embodiments, the method further comprises administering an additional therapy. This additional therapy may be any therapy known for tumors, such as surgery, chemotherapy, radiation therapy, immunotherapy, hormone therapy, or gene therapy. This additional therapy can be administered before, at the same time, or after administration of the method as described above.

In certain preferred embodiments, the subject is a mammal, such as a human.

In certain preferred embodiments, the tumor is selected from colorectal cancer, gastric cancer, lung cancer, liver cancer, ovarian cancer, endometrial cancer, cervical cancer, melanoma, breast cancer, kidney cancer, pancreatic cancer, lymphoma, osteogenic sarcoma, prostate cancer, glioma, neuroblastoma, tongue cancer, nasopharyngeal cancer, squamous cell carcinoma of nasal septum, pharyngeal squamous cell carcinoma, squamous cell carcinoma of submandibular gland, laryngeal cancer, thyroid cancer, thyroid ductal carcinoma and bladder cancer. In certain preferred embodiments, the tumor is selected from lung cancer, esophageal cancer, ovarian cancer, endometrial cancer, pancreatic cancer, tongue cancer, kidney cancer, prostate cancer, nasopharyngeal cancer, and bladder cancer.

In a third aspect, the present invention also relates to a pharmaceutical composition, which comprises the CVB1 and/or modified form thereof as defined in the first or second aspect, or the nucleic acid molecule as defined in the first or second aspect.

In certain preferred embodiments, the pharmaceutical composition may be in any form known in the medical art. For example, the pharmaceutical composition may be a tablet, pill, suspension, emulsion, solution, gel, capsule, powder, granule, elixir, lozenge, suppository, injection (including injection liquid, lyophilized powder), and other forms. In some embodiments, the pharmaceutical composition is an injection liquid or a lyophilized powder.

In certain preferred embodiments, the pharmaceutical composition further comprises a pharmaceutically acceptable carrier or excipient. In certain preferred embodiments, the pharmaceutical composition comprises a stabilizer.

In certain preferred embodiments, the pharmaceutical composition optionally further comprises an additional pharmaceutically active agent. In a preferred embodiment, the additional pharmaceutically active agent is a drug with anti-tumor activity, such as an additional oncolytic virus, chemotherapeutic agent or immunotherapeutic agent.

In certain preferred embodiments, the pharmaceutical composition is used to treat a tumor in a subject.

In certain preferred embodiments, the subject is a mammal, such as a human.

In certain preferred embodiments, the tumor is selected from colorectal cancer, gastric cancer, lung cancer, liver cancer, ovarian cancer, endometrial cancer, cervical cancer, melanoma, breast cancer, kidney cancer, pancreatic cancer, lymphoma, osteogenic sarcoma, prostate cancer, glioma, neuroblastoma, tongue cancer, nasopharyngeal cancer, squamous cell carcinoma of nasal septum, pharyngeal squamous cell carcinoma, squamous cell carcinoma of submandibular gland, laryngeal cancer, thyroid cancer, thyroid ductal carcinoma and bladder cancer. In certain preferred embodiments, the tumor is selected from lung cancer, esophageal cancer, ovarian cancer, endometrial cancer, pancreatic cancer, tongue cancer, kidney cancer, prostate cancer, nasopharyngeal cancer, and bladder cancer.

In a fourth aspect, the invention also relates to the CVB1 and/or modified form thereof as defined in the first or second aspect, or the nucleic acid molecule as defined in the first or second aspect, for use as a medicament.

In a fifth aspect, the present invention provides a modified CVB1 which has a substitution of an internal ribosome entry site (IRES) sequence in a 5′UTR with an internal ribosome entry site sequence of human rhinovirus 2 (HRV2) as compared to a wild-type CVB1.

In certain preferred embodiments, the internal ribosome entry site sequence of human rhinovirus 2 (HRV2) is shown in SEQ ID NO:2.

In certain preferred embodiments, the genomic sequence of the wild-type CVB1 has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% to the nucleotide sequence as shown in SEQ ID NO: 12. In certain preferred embodiments, the genomic sequence of the wild-type CVB1 is the nucleotide sequence as shown in SEQ ID NO:12.

In certain preferred embodiments, the cDNA sequence of the wild-type CVB1 has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% to the nucleotide sequence as shown in SEQ ID NO: 1. In certain preferred embodiments, the cDNA sequence of the wild-type CVB1 is the nucleotide sequence as shown in SEQ ID NO:1.

In certain preferred embodiments, the modified CVB1 also contains an exogenous nucleic acid.

In certain preferred embodiments, the exogenous nucleic acid encodes a cytokine (e.g., GM-CSF, preferably human GM-CSF), or an anti-tumor protein or polypeptide (e.g., scFv against PD-1 or PD-L1, preferably, scFv against human PD-1 or PD-L1). In certain preferred embodiments, the exogenous nucleic acid is inserted between 5′UTR and VP4 gene, or between VP1 gene and 2A gene of a genome of the modified CVB1.

In certain preferred embodiments, the exogenous nucleic acid comprises a target sequence of microRNA (miRNA) (e.g., miR-133 or miR-206). In certain preferred embodiments, the target sequence of microRNA is inserted in a 3′ untranslated region (3′UTR) of a genome of the modified CVB1.

In certain preferred embodiments, the exogenous nucleic acid includes a target sequence of one or more (e.g., 2, 3 or 4) microRNAs as described above. In certain preferred embodiments, the exogenous nucleic acid comprises the target sequence of miR-133 and/or miR-206. In certain preferred embodiments, the target sequence of miR-133 is shown in SEQ ID NO:3. In certain preferred embodiments, the target sequence of miR-206 is shown in SEQ ID NO:4.

In certain preferred embodiments, the modified CVB1 comprises at least one insertion of the exogenous nucleic acid as described above.

In certain preferred embodiments, the genomic sequence of the modified CVB1 has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% to the nucleotide sequence as shown in SEQ ID NO: 13. In certain preferred embodiments, the genomic sequence of the modified CVB1 is the nucleotide sequence as shown in SEQ ID NO: 13.

In certain preferred embodiments, the cDNA sequence of the modified CVB1 has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% to the nucleotide sequence as shown in SEQ ID NO: 8. In certain preferred embodiments, the cDNA sequence of the modified CVB1 is the nucleotide sequence as shown in SEQ ID NO: 8.

In certain preferred embodiments, the modified CVB1 is used for treating a tumor in a subject, or for manufacture of a medicament for treating a tumor in a subject.

In certain preferred embodiments, the tumor is selected from colorectal cancer, gastric cancer, lung cancer, liver cancer, ovarian cancer, endometrial cancer, cervical cancer, melanoma, breast cancer, kidney cancer, pancreatic cancer, lymphoma, osteogenic sarcoma, prostate cancer, glioma, neuroblastoma, tongue cancer, nasopharyngeal cancer, squamous cell carcinoma of nasal septum, pharyngeal squamous cell carcinoma, squamous cell carcinoma of submandibular gland, laryngeal cancer, thyroid cancer, thyroid ductal carcinoma and bladder cancer. In certain preferred embodiments, the tumor is selected from lung cancer, esophageal cancer, ovarian cancer, endometrial cancer, pancreatic cancer, tongue cancer, kidney cancer, prostate cancer, nasopharyngeal cancer, and bladder cancer. In certain preferred embodiments, the tumor is thyroid cancer.

In certain preferred embodiments, the subject is a mammal, such as a human.

In a sixth aspect, the present invention provides a nucleic acid molecule comprising a sequence selected from the following:

(1) a genomic sequence or cDNA sequence of the modified CVB1 as described in the fifth aspect; and

(2) a complementary sequence of the genomic sequence or cDNA sequence.

In certain preferred embodiments, the nucleic acid molecule consists of the genomic sequence or cDNA sequence of the modified CVB1 as described above, or a complementary sequence of the genomic sequence or cDNA sequence.

In certain preferred embodiments, the nucleic acid molecule has a genomic sequence of the modified CVB1 as described above. In certain preferred embodiments, the nucleic acid molecule is RNA. In certain preferred embodiments, the nucleic acid molecule has the nucleotide sequence as shown in SEQ ID NO: 13.

In certain preferred embodiments, the nucleic acid molecule is a vector (e.g., cloning vector or expression vector) comprising a genomic sequence or cDNA sequence of the modified CVB1 as described herein, or a complementary sequence of the genomic sequence or cDNA sequence. In certain preferred embodiments, the nucleic acid molecule is a vector (e.g., cloning vector or expression vector) comprising a cDNA sequence of the modified CVB1 as described herein, or a complementary sequence of the cDNA sequence.

In certain preferred embodiments, the nucleic acid molecule comprises a complementary sequence of the genomic sequence of the modified CVB1. In certain preferred embodiments, the complementary sequence is complementary to a nucleotide sequence selected from the following:

(1) a nucleotide sequence as shown in SEQ ID NO: 13; and

(2) a nucleotide sequence having a sequence identity of at least 70%, at least 80%, at least 85%, at least 90%, at least 91%, at least 92%, at least 93%, at least 94%, at least 95%, at least 96%, at least 97%, at least 98%, at least 99%, or 100% to the nucleotide sequence as shown in SEQ ID NO: 13.

In certain preferred embodiments, the nucleic acid molecule comprises a complementary sequence of the cDNA sequence of the modified CVB1 as described above. In certain preferred embodiments, the complementary sequence is complementary to a nucleotide sequence selected from the following:

(1) a nucleotide sequence as shown in SEQ ID NO: 8; and

(2) a nucleotide sequence having a sequence identity of at least 70%, at least 80%, at least 85%, at least 90%, at least 91%, at least 92%, at least 93%, at least 94%, at least 95%, at least 96%, at least 97%, at least 98%, at least 99%, or 100% to the nucleotide sequence as shown in SEQ ID NO: 8.

In certain preferred embodiments, the nucleic acid molecule is used for treating a tumor in a subject, or for manufacture of a medicament for treating a tumor in a subject.

In certain preferred embodiments, the tumor is selected from colorectal cancer, gastric cancer, lung cancer, liver cancer, ovarian cancer, endometrial cancer, cervical cancer, melanoma, breast cancer, kidney cancer, pancreatic cancer, lymphoma, osteogenic sarcoma, prostate cancer, glioma, neuroblastoma, tongue cancer, nasopharyngeal cancer, squamous cell carcinoma of nasal septum, pharyngeal squamous cell carcinoma, squamous cell carcinoma of submandibular gland, laryngeal cancer, thyroid cancer, thyroid ductal carcinoma and bladder cancer. In certain preferred embodiments, the tumor is selected from lung cancer, esophageal cancer, ovarian cancer, endometrial cancer, pancreatic cancer, tongue cancer, kidney cancer, prostate cancer, nasopharyngeal cancer, and bladder cancer. In certain preferred embodiments, the tumor is thyroid cancer.

In certain preferred embodiments, the subject is a mammal, such as a human.

In a seventh aspect, the invention relates to a pharmaceutical composition comprising the modified CVB1 according to the fifth aspect, or the nucleic acid molecule according to the sixth aspect.

In certain preferred embodiments, the pharmaceutical composition may be in any form known in the medical art. For example, the pharmaceutical composition may be tablet, pill, suspension, emulsion, solution, gel, capsule, powder, granule, elixir, lozenge, suppository, injection (including injection liquid, lyophilized powder) and other forms. In some embodiments, the pharmaceutical composition is an injection liquid or a lyophilized powder.

In certain preferred embodiments, the pharmaceutical composition further comprises a pharmaceutically acceptable carrier or excipient. In certain preferred embodiments, the pharmaceutical composition comprises a stabilizer.

In certain preferred embodiments, the pharmaceutical composition optionally further comprises an additional pharmaceutically active agent. In a preferred embodiment, the additional pharmaceutically active agent is a drug with anti-tumor activity, such as an additional oncolytic virus, chemotherapeutic agent or immunotherapeutic agent.

In certain preferred embodiments, the pharmaceutical composition is used for treating a tumor in a subject.

In certain preferred embodiments, the tumor is selected from colorectal cancer, gastric cancer, lung cancer, liver cancer, ovarian cancer, endometrial cancer, cervical cancer, melanoma, breast cancer, kidney cancer, pancreatic cancer, lymphoma, osteogenic sarcoma, prostate cancer, glioma, neuroblastoma, tongue cancer, nasopharyngeal cancer, squamous cell carcinoma of nasal septum, pharyngeal squamous cell carcinoma, squamous cell carcinoma of submandibular gland, laryngeal cancer, thyroid cancer, thyroid ductal carcinoma and bladder cancer. In certain preferred embodiments, the tumor is selected from lung cancer, esophageal cancer, ovarian cancer, endometrial cancer, pancreatic cancer, tongue cancer, kidney cancer, prostate cancer, nasopharyngeal cancer, and bladder cancer. In certain preferred embodiments, the tumor is thyroid cancer.

In certain preferred embodiments, the subject is a mammal, such as a human.

In an eighth aspect, the present invention also relates to use of the modified CVB1 according to the fifth aspect, or the nucleic acid molecule according to the sixth aspect, for treating a tumor in a subject, or for manufacture of a medicament for treating a tumor in a subject.

In certain preferred embodiments, the tumor is selected from colorectal cancer, gastric cancer, lung cancer, liver cancer, ovarian cancer, endometrial cancer, cervical cancer, melanoma, breast cancer, kidney cancer, pancreatic cancer, lymphoma, osteogenic sarcoma, prostate cancer, glioma, neuroblastoma, tongue cancer, nasopharyngeal cancer, squamous cell carcinoma of nasal septum, pharyngeal squamous cell carcinoma, squamous cell carcinoma of submandibular gland, laryngeal cancer, thyroid cancer, thyroid ductal carcinoma and bladder cancer. In certain preferred embodiments, the tumor is selected from lung cancer, esophageal cancer, ovarian cancer, endometrial cancer, pancreatic cancer, tongue cancer, kidney cancer, prostate cancer, nasopharyngeal cancer, and bladder cancer. In certain preferred embodiments, the tumor is thyroid cancer.

In certain preferred embodiments, the subject is a mammal, such as a human.

In a ninth aspect, the present invention also relates to a method of treating a tumor, which comprises a step of administering to a subject in need thereof an effective amount of the modified CVB1 as described in the fifth aspect, or an effective amount of the nucleic acid molecule as described in the sixth aspect.

In certain preferred embodiments, the tumor is selected from colorectal cancer, gastric cancer, lung cancer, liver cancer, ovarian cancer, endometrial cancer, cervical cancer, melanoma, breast cancer, kidney cancer, pancreatic cancer, lymphoma, osteogenic sarcoma, prostate cancer, glioma, neuroblastoma, tongue cancer, nasopharyngeal cancer, squamous cell carcinoma of nasal septum, pharyngeal squamous cell carcinoma, squamous cell carcinoma of submandibular gland, laryngeal cancer, thyroid cancer, thyroid ductal carcinoma and bladder cancer. In certain preferred embodiments, the tumor is selected from lung cancer, esophageal cancer, ovarian cancer, endometrial cancer, pancreatic cancer, tongue cancer, kidney cancer, prostate cancer, nasopharyngeal cancer, and bladder cancer. In certain preferred embodiments, the tumor is thyroid cancer.

In certain preferred embodiments, the subject is a mammal, such as a human.

Beneficial effects of the invention

Compared with the prior art, the technical solution of the present invention has at least the following beneficial effects:

The inventors of the present application have found for the first time that CVB1 has a broad-spectrum tumor-killing activity. Based on this finding, the present invention further provides an oncolytic virus based on CVB1, which has higher tumor killing activity and tumor specificity, thus can be used alone in the treatment of tumors, and can also be used as an auxiliary method for traditional tumor treatment, or as a treatment method when other treatment methods are lacking.

The CVB1 or modified form thereof according to the present invention has little or no effect on normal cells, and can be safely administered to a subject (such as a human). Therefore, the CVB1 modified form thereof according to the present invention has great clinical value.

The embodiments of the present invention will be described in detail below in conjunction with the drawings and examples, but those skilled in the art will understand that the following drawings and examples are only used to illustrate the present invention, not to limit the scope of the present invention. The various objects and advantageous aspects of the invention will become apparent to those skilled in the art from the following detailed description of the drawings and preferred embodiments.

### BRIEF DESCRIPTION OF THE DRAWINGS

FIGS. 1A to 1D show the micrographs of the in vitro killing experiments in Example 2 of wild type CVB1 on human pancreatic ductal epithelial cell line hTERT-HPNE, human nasopharyngeal carcinoma cell line CNE, human liver cancer cell line HepG2, human endometrial cancer cell line Ishikawa, human breast cancer cell line BT-474, human non-small cell lung cancer cell line EBC-1, human laryngeal cancer cell line HEp-2, human tongue cancer cell line SCC-25, human colorectal cancer cell line HT-29, human ovarian cancer cell line A2780, human pancreatic cancer cell line AsPC-1, and human prostate cancer cell line DU145, in which MOCK indicates cells that were not infected with the virus. The results show that after 72 hours of infection with a multiplicity of infection (MOI) of 1, CVB1 showed a significant oncolytic effect on human tumor cell lines CNE, HepG2, Ishikawa, BT-474, EBC-1, HEp-2, SCC-25, HT-29, A2780, AsPC-1 and DU145, but had no effect on human non-tumor cell hTERT-HPNE.

FIG. 2 shows an electropherogram of one sample of the wild-type CVB1 virus genomic RNA obtained by the in vitro transcription method in Example 2.

FIG. 3 shows the killing effect of the wild-type CVB1 virus genomic RNA on human cervical cancer cell line Hela in Example 2. The results show that the Hela cells transfected with the CVB1 genomic RNA were almost completely lysed and died 48 hours after the transfection.

FIGS. 4A to 41 show the results of in vivo anti-tumor experiments of the wild-type CVB1 against human breast cancer cell line BcaP37 (A), human non-small cell lung cancer cell line A549 (B) and SPC-A-1 (C), human Burkitt's lymphoma cell lines Raji (D), human endometrial cancer cell lines Ishikawa (E) and HEC-1-B (F), human cervical cancer cell lines Hela (G) and C-33A (H), and human glioma cell line GBM (I) in Example 3 of the present invention. The results show that in the challenge experiment groups, 10⁶ TCID50 per tumor mass of CVB1 were injected intratumorally every two days. After 5 treatments in total, the growth of the tumors formed by subcutaneous inoculation of BcaP37, A549, SPC-A-1, Raji, Ishikawa, HEC-1-B, Hela, C-33A or GBM cells in SCID mice significantly slowed down and arrested, and the tumors were even lysed and disappeared. In contrast, the tumors of the negative group (CTRL) without treatment of oncolytic virus maintained the normal growth, and their tumor volumes were significantly larger than those in the challenge groups.

FIG. 5 shows the results of in vivo anti-tumor experiments of CVB1-WT, CVB1-HRV2, CVB1-miR133&206T, CVB1-GM-CSF, and CVB1-Anti-PD-1 against human glioma cell line GBM in Example 3. The results showed that, in the challenge experimental groups, 10⁶ TCID50 per tumor mass of CVB1 or modified forms thereof were injected intratumorally every two days. After 5 treatments in total for 10 days, the growth of the tumors formed by subcutaneous inoculation of GBM cells in SCID mice arrested, and the tumors were even lysed and disappeared. In contrast, the tumors of the negative group (CTRL) without treatment of oncolytic virus maintained the normal growth, and their tumor volumes were significantly larger than those in the challenge groups.

### SEQUENCE INFORMATION

Information of parts of sequences involved in the present invention is provided in Table 1 below.

TABLE 1
Description of the sequences

| SEQ ID NO: | Description |
|---|---|
| 1 | cDNA sequence of wild type CVB1 (CVB1-WT) |
| 2 | RNA sequence of internal ribosome entry site |
| | sequence of human rhinovirus 2 (HRV2) |
| 3 | RNA sequence of miR-133 target sequence |
| 4 | RNA sequence of miR-206 target sequence |
| 5 | RNA sequence of tandem sequence of miR-133 target |
| | sequence and miR-206 target sequence |
| 6 | DNA sequence of human granulocyte-macrophage |
| | colony stimulating factor (GM-CSF) gene |
| 7 | DNA sequence of anti-PD-1 single chain antibody |
| | (Anti-PD-1 scFv) |
| 8 | cDNA sequence of the modified form of CVB1 |
| | (CVB1-HRV2) |
| 9 | cDNA sequence of the modified form of CVB1 |
| | (CVB1-miR133&206T) |
| 10 | cDNA sequence of the modified form of CVB1 |
| | (CVB1-GM-CSF) |
| 11 | cDNA sequence of the modified form of CVB1 |
| | (CVB1-Anti-PD1) |
| 12 | Genomic sequence of wild type CVB1 (CVB1-WT) |
| 13 | Genomic sequence of the modified form of CVB1 |
| | (CVB1-HRV2) |
| 14 | Genomic sequence of the modified form of CVB1 |
| | (CVB1-miR133&206T) |
| 15 | Genomic sequence of the modified form of CVB1 |
| | (CVB1-GM-CSF) |
| 16 | Genomic sequence of modified form of CVB1 |
| | (CVB1-Anti-PD1) |
| 17 | DNA sequence of miR-133 target sequence |
| 18 | DNA sequence of miR-206 target sequence |
| 19 | DNA sequence of tandem sequence of miR-133 target |
| | sequence and miR-206 target sequence |
| 20 | DNA sequence of internal ribosome entry site |
| | sequence of human rhinovirus 2 (HRV2) |

### Specific Models for Carrying Out the Invention

The present invention will now be described with reference to the following examples intended to illustrate the invention (not to limit the invention).

Unless otherwise specified, the molecular biology experimental methods and immunoassays used in the present invention basically referred to J. Sambrook et al., Molecular Cloning: A Laboratory Manual, 2^{nd} Edition, Cold Spring Harbor Laboratory Press, 1989, and F M Ausubel et al., Short Protocols in Molecular Biology, 3^{rd} Edition, John Wiley & Sons, Inc., 1995. The use of restriction enzymes was in accordance with the conditions recommended by the product manufacturers. If no specific conditions were indicated in the examples, the conventional conditions or the conditions recommended by the manufacturers should be followed. The used reagents or instruments, of which manufacturers were not given, were all conventional products that were commercially available. Those skilled in the art know that the examples describe the present invention by way of example, and are not intended to limit the claimed scope of the invention. All publications and other references mentioned herein are incorporated by reference in their entirety.

### Example 1: Acquisition and Preparation of CVB1 and Modified Forms Thereof

1.1 Isolation of Enterovirus CVB1 from Clinical Specimens of Patients

(1) The pharyngeal and anal swabs of patients were from the Center for Disease Control and Prevention of Xiamen City, China; African green monkey kidney cells (Vero cells; ATCC® Number: CCL-81™) were preserved by the National Institute of Diagnostics and Vaccine Development in Infectious Diseases, Xiamen University, China, and were cultured in MEM medium supplemented with 10% fetal bovine serum, glutamine, penicillin and streptomycin.

(2) Sample processing: the pharyngeal swabs and anal swabs of patients were sufficiently agitated in a specimen preservation solution to wash off the virus and virus-containing cells adhering to the swabs, and then the specimen preservation solution was subjected to high speed centrifugation at 4000 rpm and 4° C. for 30 min;

(3) Inoculation and observation:

A. Vero cells were plated in a 24-well plate with 1×10⁵ cells/well. The growth medium (MEM medium, containing 10% fetal bovine serum, as well as glutamine, penicillin and streptomycin) was aspirated, and 1 mL of maintenance medium (MEM medium, containing 2% fetal calf serum, as well as glutamine, penicillin and streptomycin) was added in each well. Then except the negative control wells, each well was inoculated with 50 μL of the sample supernatant, and cultured in an incubator at 37° C., 5% CO₂.

B. The cells were observed under a microscope every day for one week, and the occurrence of specific cytopathic effect (CPE) in the inoculated wells was recorded.

C. If the enterovirus-specific cytopathic effect appeared in the cells in the inoculated wells within 7 days, the cells and supernatant were collected and frozen at −80° C.; if no CPE appeared after 7 days, the cells were subjected to blind passage.

D. If CPE appeared within 6 blind passages, the cells and supernatant were collected and frozen at −80° C.; If CPE did not appear after 6 blind passages, the cells were determined as negative.

(4) Virus Isolation and Cloning:

The viruses isolated from the clinical specimens were identified by RT-PCR (Hou et al., Virus Res 2015, 205: 41-44) and specific antibody-based enzyme-linked immunospot assay (ELISPOT) (Yang et al. Clin Vaccine Immunol 2014, 21(3): 312-320), and Coxsackievirus B1 positive cultures were selected and subjected to at least 3 cloning experiments. The virus clones obtained by the limiting dilution method in each experiment were also identified by RT-PCR and ELISPOT, and Coxsackievirus B1 positive clones were selected and subjected to the next round of cloning. A single strain of Coxsackievirus B1 with strong growth viability were selected as a candidate oncolytic virus strain.

1.2 Obtaining Rescued Strains of CVB1 and Modified Forms Thereof Based on Infectious Cloning and Reverse Genetics Technology

This example used the wild-type CVB1 (SEQ ID NO: 1) as an example to show how to obtain CVB1 and modified forms thereof used in the present invention by reverse genetics technology. The specific method was as follows.

(1) Construction of viral infectious clones: The cDNA sequence of the wild-type CVB1 (named CVB1-WT) was shown in SEQ ID NO:1, and its genomic RNA sequence was shown in SEQ ID NO:12; or the gene insertion or substitution based on the cDNA of the wild-type CVB1 (SEQ ID NO:1), comprising:

Modified form 1: The internal ribosome entry site sequence of the wild-type CVB1 was replaced with the internal ribosome entry site sequence of human rhinovirus 2 (which has a DNA sequence shown in SEQ ID NO: 20), to obtain the cDNA (SEQ ID NO: 8) of a recombinant virus (named as CVB1-HRV2), which has a genomic RNA sequence shown as SEQ ID NO: 13;

Modified form 2: The tandem sequence (which has a DNA sequence shown in SEQ ID NO: 19) of the miR-133 target sequence (which has a DNA sequence shown in SEQ ID NO: 17) and the miR-206 target sequence (which has a DNA sequence shown in SEQ ID NO: 18) was inserted between 7303-7304 bp of the 3′ untranslated region of the cDNA (SEQ ID NO: 1) of the wild-type CVB1, to obtain the cDNA (SEQ ID NO: 9) of a recombinant virus (named CVB1-miR133&206T), which has a genomic RNA sequence shown as SEQ ID NO: 14;

Modified form 3: The human granulocyte-macrophage colony stimulating factor (GM-CSF) gene (SEQ ID NO: 6) was inserted between the VP1 gene and 2A gene of the cDNA (SEQ ID NO: 1) of wild-type CVB1 to obtain the cDNA (SEQ ID NO: 10) of a recombinant virus (named CVB1-GM-CSF), which has a genomic RNA sequence shown as SEQ ID NO: 15;

Modified form 4: The sequence (SEQ ID NO: 7) encoding the single chain antibody against human programmed death receptor 1 (Anti-PD-1 scFv) was inserted into the VP1 gene and the 2A gene of wild-type CVB1 to obtain the cDNA (SEQ ID NO: 11) of a recombinant virus (named CVB1-Anti-PD-1), which has a genomic RNA sequence shown as SEQ ID NO: 16.

The cDNA sequences (SEQ ID NOs: 1, 8-11) of the above five oncolytic viruses were sent to the gene synthesis company (Shanghai Shenggong Bioengineering Co., Ltd.) for full gene synthesis, and ligated into pSVA plasmids (Hou et al. Human, Virus Res 2015, 205: 41-44), thereby obtaining infectious cloning plasmids of the CVB1 or its modified forms (i.e., CVB1-WT, CVB1-HRV2, CVB1-miR133&206T, CVB1-GM-CSF and CVB1-Anti-PD-1).

(2) Plasmid mini-kit and E coli. DH5a competent cells were purchased from Beijing Tiangen Biochemical Technology Co., Ltd.; Hela cells (ATCC® Number: CCL-2™) and human rhabdomyosarcoma cells (RD cells; ATCC® Number: CCL-i36™) were kept by National Institute of Diagnostics and Vaccine Development in Infectious Diseases, Xiamen University, China, and were cultured with DMEM and MEM media respectively, in which 10% fetal bovine serum as well as glutamine, penicillin and streptomycin were added; transfection reagents Lipofactamine2000 and Opti-MEM were purchased from Thermo Fisher Scientific Company.

(3) The infectious cloning plasmids containing the cDNA sequences of the above five oncolytic viruses were transformed into E coli DH5a competent cells, the monoclonal strains were picked out and shaken after the outgrowth of clones, and the plasmids were extracted using the plasmid mini-kit, and then sent to the company (Shanghai Biotech Engineering Co., Ltd.) for sequencing analysis.

(4) The infectious cloning plasmids with correct sequence and the helper plasmid pAR3126 were co-transfected into the cells to rescue virus (Hou et al. Virus Res 2015, 205: 41-44). Hela cells were first transfected according to the instructions of the transfection reagent; then observed under a microscope. When CPE appeared in Hela cells, the cells and culture supernatant were harvested, and inoculated with RD cells followed by passaging and culturing. The rescued strains obtained thereby can be used as the candidate strain of oncolytic virus.

### Example 2: In Vitro Anti-Tumor Experiment of CVB1 and its Modified Forms

2.1 Viruses and Cell Lines as Used

(1) Viruses: In this example, the CVB1-WT (SEQ ID NO: 12), CVB1-HRV2 (SEQ ID NO: 13), CVB1-miR133&206T (SEQ ID NO: 14), CVB1-GM-CSF (SEQ ID NO: 15) and CVB1-Anti-PD-1 (SEQ ID NO: 16) as provided in Example 1 and a strain of wild-type Coxsackievirus B type 3 (hereinafter referred to as: CVB3-WT; GenBank database accession number: KY286529.1) were used.

(2) Cell lines: human rhabdomyosarcoma cell RD (ATCC® Number: CCL-136™); human colorectal cancer cell lines SW1116 (ATCC® Number: CCL-233™), SW480 (ATCC® Number: CCL-228™) and HT-29 (ATCC® Number: HTB-38™); human gastric cancer cell lines AGS (ATCC® Number: CRL-1739™), SGC7901 (CCTCC deposit number: GDC150), BGC823 (CCTCC deposit number: GDC151) and NCI-N87 (ATCC® Number: CRL-5822™); human esophageal cancer cell line TE-1 (purchased from the Cell Resource Center, Shanghai Institute for Biological Sciences, Chinese Academy of Sciences, No. 3131C0001000700089); human small cell lung cancer cell line DMS114 (ATCC® Number: CRL-2066™); human non-small cell lung cancer cell lines SPC-A-1 (CCTCC deposit number: GDC050), NCI-H1975 (ATCC® Number: CRL-5908™), NCI-H1299 (ATCC® Number: CRL-5803™), A549 (ATCC® Number: CCL-185™), NCI-H661 (ATCC® Number: HTB-183™), EBC-1 (Thermo Fisher Scientific, Catalog #: 11875101) and NCI-H1703 (ATCC® Number: CRL-5889™); human liver cancer cell lines C3A (ATCC® Number: CRL-10741™), HepG2 (ATCC® Number: HB-8065™), SMMC7721 (purchased from the Basic Medical Cell Center, Institute of Basic Medical Sciences, Chinese Academy of Medical Sciences, Number: 3111C0001CCC000087), BEL7402 (CCTCC deposit number: GDC035), BEL7404 (purchased from the Cell Resource Center, Shanghai Institutes for Biological Sciences, Chinese Academy of Sciences, number: 3131C0001000700064), Huh? (CCTCC deposit number: GDC134) and PLC/PRF/5 (ATCC® Number: CRL-8024™); human ovarian cancer cell lines SKOV3 (ATCC® Number: HTB-77™) and Caov3 (ATCC® Number: HTB-75™); human endometrial cancer cell lines Hec-1-A (ATCC® Number: HTB-112™), Hec-1-B (ATCC® Number: HTB-113™) and Ishikawa (ECACC No. 99040201); human cervical cancer cell lines Hela (ATCC® Number: CCL-2™), Caski (ATCC® Number: CRL-1SSO™) and C-33A (ATCC® Number: HTB-31™); human melanoma cell lines SK-MEL-1 (ATCC® Number: HTB-67™) and MeWo (ATCC® Number: HTB-65™); human breast cancer cell lines BcaP37 (CCTCC deposit number: GDC206), BT-474 (ATCC® Number: HTB-20™) and MDA-MB-231 (ATCC® Number: HTB-26™); human kidney cancer cell lines A-498 (ATCC® Number: HTB-44™) and 786-0 (ATCC® Number: CRL-1932™); human pancreatic cancer cell lines Capan-2 (ATCC® Number: HTB-80™), AsPC-1 (ATCC® Number: CRL-1682™), SW1990 (ATCC® Number: CRL-2172™), HPAF-2 (ATCC® Number: CRL-1997™) and CFPAC-1 (ATCC® Number: CRL-1918™); human osteosarcoma cell line U2OS (ATCC® Number: HTB-96™); human prostate cancer cell lines DU145 (ATCC® Number: HTB-81™) and LNCap (ATCC® Number: CRL-1740™); human neuroglioma cell line GBM (primary tumor cell line isolated from a patient tumor tissue); human neuroblastoma cell line SH-SYSY (ATCC® Number: CRL-2266™); human tongue squamous carcinoma cell lines CAL27 (ATCC® Number: CRL-2095™) and SCC-25 (ATCC® Number: CRL-1628™); human nasopharyngeal carcinoma cell line CNE (purchased from the Center for Basic Medical Cells, Institute of Basic Medical Sciences, Chinese Academy of Medical Sciences, No.: 3131C0001000700013); human nasal septum squamous cell carcinoma cell line RPMI 2650 (ATCC® Number: CCL-30™); human laryngeal carcinoma cell line HEp-2 (ATCC® Number: CCL-23™); human thyroid cancer cell lines SW579 (preserved by the National Engineering Research Center for Diagnostic Reagents and Vaccines for Infectious Disease) and human thyroid ductal carcinoma cell line TT (ATCC® Number: CRL-1803™); human bladder cancer cell lines J82 (ATCC® Number: HTB-1™) and 5637 (ATCC® Number: HTB-9™); human Burkitt's lymphoma cell lines Daudi (ATCC® Number: CCL-213™) and Raji (ATCC® Number: CCL-86™); human normal cell lines including: human pancreatic ductal epithelial cell line hTERT-HPNE (ATCC® Number: CRL-4023™), human skin keratinocyte cell line HaCat (CCTCC, deposit number: GDC106), human embryonic lung fibroblast cell line MRC-5 (ATCC® Number: CCL-171™), human foreskin fibroblast cell line HFF-1 (ATCC® Number: SCRC-1041™), human prostate stromal cell line WPMY-1 (ATCC® Number: CRL-2854™), human umbilical vein endothelial cell line HUVEC (Thermo Fisher Scientific, Catalog #: C01510C) and the differentiated human liver progenitor cell line HepaRG (with the characteristics of primary hepatocytes; Thermo Fisher Scientific, Catalog #: HPRGC10). The above cells were all preserved by National Institute of Diagnostics and Vaccine Development in Infectious Diseases, Xiamen University, China. HepaRG cells were cultured in WME medium (added with 1.5% DMSO); AGS and TT were cultured in F-12K medium; SH-SYSY was cultured in DMEM:F12 (1:1) medium; CFPAC-1 was cultured in IMDM medium; RD, C-33A, EBC-1, SK-MEL-1, J82 and DU145 were cultured in MEM medium; Raji, Daudi, 5637, 786-0, TE-1, Caski, NCI-H1299, NCI-H1703, NCI-H1975, NCI-H661, SGC7901, BGC823, SW1116, HEp-2 and LNCap were cultured in RPMI-1640 medium; and other cells were cultured in DMEM medium. These mediums were all supplemented with 10% fetal bovine serum, glutamine and penicillin-streptomycin. All the above cells were cultured under standard conditions of 37° C. and 5% CO₂.

2.2 Cultivation of Virus

The RD cells were evenly plated on 10 cm cell culture plates, under the culturing conditions of MEM medium containing 10% fetal bovine serum, glutamine, penicillin and streptomycin, 37° C., 5% CO₂, and saturated humidity. When the cell confluence reached 90% or more, the cell culture medium was replaced with serum-free MEM medium, and each plate was inoculated with 10⁷ TCID50 of CVB1-WT, CVB1-HRV2, CVB1-miR133&206T, CVB1-GM-CSF or CVB1-Anti-PD-1. After 24 hours of continuous cultivation, CVB1 or its modified forms proliferated in RD cells and caused CPE in the cells. When more than 90% of the cells turned contracted and rounded, showed increased graininess, and became detached and lysed, the cells and their culture supernatants were harvested. After freeze-thawing for three cycles, the culture supernatants were collected and centrifuged to remove cell debris, under the centrifugation conditions of 4000 rpm, 10 min, and 4° C. Finally, the supernatants were filtered with 0.22 μm disposable filter (Millipore) to remove all cell debris and other impurities.

2.3 Determination of Virus Titer

The RD cells were coated in a 96-well plate with a cell density of 10⁴ cells/well. After the cells adhered, the virus solution obtained in Example 2.2 was subjected to a 10-fold gradient dilution starting at 10-fold with serum-free MEM medium. 50 μl of the diluted virus was added to the wells with cells. After 7 days, the wells where CPE appeared were monitored and recorded, followed by calculation using Karber method, in which the calculation formula was lg^{TCID50}=L−D (S−0.5), L: logarithm of the highest dilution, D: difference between logarithms of dilutions, S: sum of proportions of positive wells. The unit of TCID50 thereby calculated was TCID50/50μ1, which should be converted into TCID50/ml.

2.4 In Vitro Anti-Tumor Experiments of Viruses

The human tumor cells and normal cells were inoculated into 96-well plates at 10⁴ per well. After the cells adhered, the medium in each well was replaced with corresponding cell culture medium without serum, and viruses were inoculated at MOIs of 10, 1, 0.1 and 0.01, respectively. Then, CPE of the cells were monitored daily by a microscope.

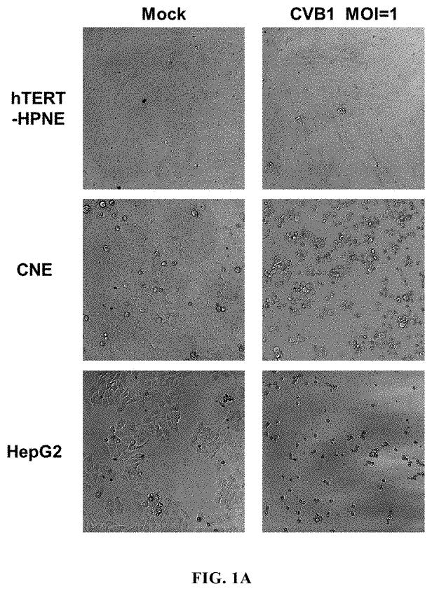

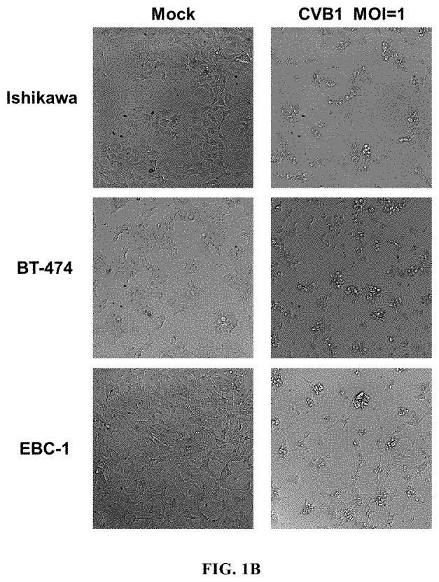

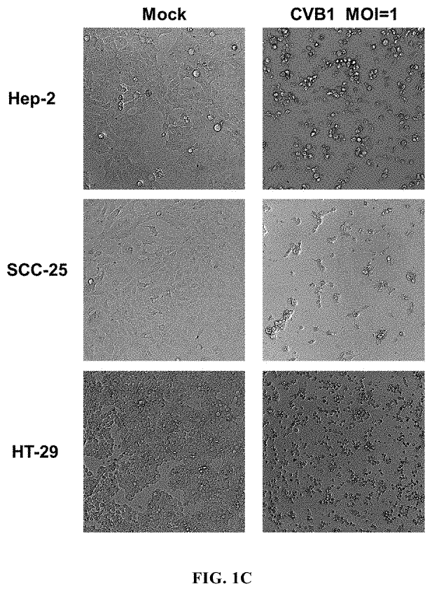

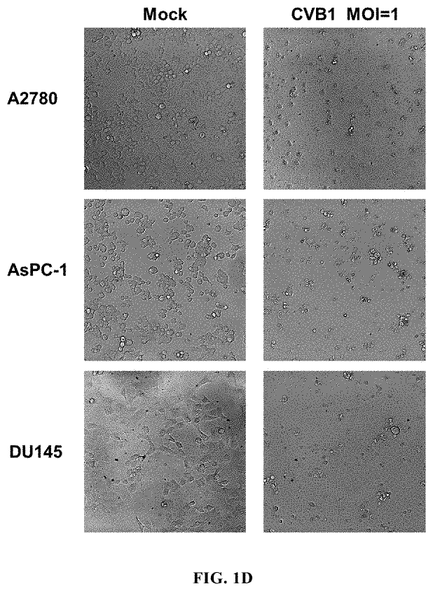

FIGS. 1A to 1D showed the micrographs of the human pancreatic ductal epithelial cell line hTERT-HPNE, human nasopharyngeal carcinoma cell line CNE, human liver cancer cell line HepG2, human endometrial cancer cell line Ishikawa, human breast cancer cell line BT-474, human non-small cell lung cancer cell line EBC-1, human laryngeal cancer cell line HEp-2, human tongue cancer cell line SCC-25, human colorectal cancer cell line HT-29, human ovarian cancer cell line A2780, human pancreatic cancer cell line AsPC-1 and human prostate cancer cell line DU145, which were not infected with viruses (negative control group, Mock) or which were treated with CVB1-WT at MOI=1 for 72 hours. The results showed that after 72 hours of infection with a multiplicity of infection (MOI) of 1, a significant reduction in the number of the tumor cells, marked shrinking and lysis and the like, were detected in the virus-infected groups; while as compared to the non-tumor cells in the Mock group, the non-tumor cells infected with the viruses showed almost no change in cell morphology. The above results indicated that CVB1 showed significant oncolytic effects on human nasopharyngeal cancer cell line CNE, human liver cancer cell line HepG2, human endometrial cancer cell line Ishikawa, human breast cancer cell line BT-474, human non-small cell lung cancer cell line EBC-1, human laryngeal cancer cell line HEp-2, human tongue cancer cell line SCC-25, human colorectal cancer cell line HT-29, human ovarian cancer cell line A2780, human pancreatic cancer cell line AsPC-1 and human prostate cancer cell line DU145, but had no effect on the non-tumor cells such as human pancreatic ductal epithelial cell line hTERT-HPNE.

Cell Counting Kit-8 (CCK-8 kit; Shanghai Biyuntian Biotechnology Co., Ltd.) was used to detect cell survival rate after 72 hours of virus infection and culture. The specific methods were as follows:

For adherent cells, the original medium in a 96-well cell culture plate was directly discarded; for suspension cells, the original medium in a 96-well cell culture plate was carefully discarded after centrifugation; and then 100 μl of fresh serum-free medium was added per well. 10 μl of CCK-8 solution was added to each of the wells inoculated with cells, and an equal amount of CCK-8 solution was also added to the blank culture medium as a negative control, followed by incubation at 37° C. in a cell culture incubator for 0.5-3 hours. The absorbance was detected at 450 nm using a microplate reader at 0.5, 1, 2, 3 hours, respectively, and the time point where the absorbance was within a suitable range was selected as a reference for cell survival rate. The CCK-8 test results of CVB1-WT for each kind of cells were shown in Table 2, where “−” indicated that the cell survival rate after virus treatment was not significantly different from that of the MOCK group; “+” indicated that after virus treatment, the cell number was reduced, the survival rate was still greater than 50% but was significantly different from that of the MOCK group; “++” indicated that the cell survival rate after virus treatment was less than 50%, and was significantly different from that of the MOCK group.

The calculation of cell survival rate is:

$$Cell\_survival \_rate ( \% ) = \frac{( {reading}_{-} {of}_{-} {test}_{-} group - {reading}_{-} {of}_{-} {negative}_{-} group )}{( {reading}_{-} {of}_{-} {positive}_{-} group - {reading}_{-} {of}_{-} {negative}_{-} group )} \times 100 \%$$

TABLE 2
Results of in vitro anti-tumor test of CVB1-WT

MOI

| Cell lines | 10 | 1 | 0.1 | 0.01 |
|---|---|---|---|---|
| RD | ++ | ++ | ++ | + |
| SW1116 | ++ | ++ | ++ | + |
| SW480 | ++ | ++ | ++ | ++ |
| HT29 | ++ | ++ | ++ | ++ |
| AGS | ++ | ++ | ++ | ++ |
| SGC7901 | ++ | ++ | ++ | + |
| BGC823 | ++ | ++ | ++ | ++ |
| NCI-N87 | ++ | ++ | ++ | − |
| TE-1 | ++ | ++ | ++ | + |
| DMS114 | ++ | ++ | ++ | ++ |
| SPC-A-1 | ++ | ++ | ++ | ++ |
| NCI-H1975 | ++ | ++ | ++ | + |
| NCI-H1299 | ++ | ++ | ++ | ++ |
| A549 | ++ | ++ | ++ | + |
| NCI-H661 | ++ | ++ | ++ | ++ |
| EBC-1 | ++ | ++ | ++ | ++ |
| NCI-H1703 | ++ | ++ | ++ | + |
| C3A | ++ | ++ | ++ | ++ |
| HepG2 | ++ | ++ | ++ | ++ |
| SMMC7721 | ++ | ++ | ++ | ++ |
| BEL7404 | ++ | ++ | ++ | ++ |
| BEL7402 | ++ | ++ | ++ | ++ |
| Huh7 | ++ | ++ | ++ | ++ |
| PLC/PRF/5 | ++ | ++ | ++ | ++ |
| SKOV3 | ++ | ++ | + | − |
| CaOV3 | ++ | ++ | ++ | − |
| HEC-1-A | ++ | ++ | ++ | + |
| HEC-1-B | ++ | ++ | ++ | ++ |
| Ishikawa | ++ | ++ | ++ | ++ |
| Hela | ++ | ++ | ++ | ++ |
| CaSki | ++ | ++ | ++ | ++ |
| C-33A | ++ | ++ | ++ | ++ |
| SK-MEL-1 | ++ | ++ | ++ | + |
| MeWo | ++ | ++ | + | − |
| BcaP37 | ++ | ++ | ++ | ++ |
| BT-474 | ++ | ++ | ++ | ++ |
| MDA-MB-231 | ++ | ++ | + | − |
| A498 | ++ | ++ | ++ | + |
| 786-O | ++ | ++ | + | − |
| Capan-2 | ++ | ++ | + | − |
| HPAF-2 | ++ | ++ | + | + |
| AsPC-1 | ++ | ++ | ++ | ++ |
| SW1990 | ++ | ++ | ++ | + |
| CFPAC-1 | ++ | ++ | ++ | + |
| U2OS | ++ | + | − | − |
| DU145 | ++ | ++ | ++ | ++ |
| LNCap | ++ | ++ | ++ | + |
| GBM | ++ | ++ | ++ | ++ |
| SH-SY5Y | ++ | ++ | ++ | ++ |
| CAL27 | ++ | ++ | ++ | + |
| SCC-25 | ++ | ++ | ++ | ++ |
| CNE | ++ | ++ | ++ | + |
| RPMI 2650 | ++ | ++ | + | + |
| HEp-2 | ++ | ++ | ++ | ++ |
| TT | ++ | ++ | ++ | − |
| SW579 | ++ | + | − | − |
| J82 | ++ | + | − | − |
| 5637 | ++ | ++ | ++ | ++ |
| Daudi | ++ | ++ | + | − |
| Raji | ++ | ++ | + | − |
| hTERT-HPNE | + | − | − | − |
| differentiated RG | + | − | − | − |
| Hacat | − | − | − | − |
| MRC-5 | − | − | − | − |
| HFF-1 | − | − | − | − |
| wpmy-1 | − | − | − | − |
| HUVEC | − | − | − | − |

Note:
“−” indicated that there was no significant difference in cell survival rate between virus treatment group and MOCK group;
“+” indicated that after virus treatment, the number of cells was reduced, the survival rate was greater than 50% but was significantly different from that of MOCK group;
“++” indicated that the cell survival rate after virus treatment was less than 50%, and was significantly different from that of the MOCK group.

It can be seen from Table 2 that CVB1-WT had killing effect on most of the detected tumor cells. In particular, the virus had significant killing effects on colorectal cancer cell lines, gastric cancer cell lines, lung cancer cell lines, liver cancer cell lines, cervical cancer cell lines, endometrial cancer cell lines, pancreatic cancer cell lines, prostate cancer cell lines, nasopharyngeal cancer cell lines, tongue cancer cell lines, laryngeal cancer cell lines, glioma cell lines and neuroblastoma cell lines. On the other hand, the virus was substantially non-toxic to the non-tumor cell lines tested, including the human embryonic lung fibroblast cell line MRC-5, human foreskin fibroblast cell line HFF-1, human skin keratinocyte cell line HaCat, human prostate stromal cell line WPMY-1, and human umbilical vein endothelial cell line HUVEC, except that it had certain toxicity to human normal pancreatic ductal epithelial cell line hTERT-HPNE and the differentiated human liver progenitor cell line HepaRG at MOI=10.

It is particularly worth noting that although CVB1-WT and the wild-type Coxsackievirus B type 3 strain (CVB3-WT; GenBank database accession number: KY286529.1) reported to have certain killing activity on specific tumors, belong to Coxsackie viruses, the genome-wide nucleotide homology between the two was only 72.8%, and the nucleotide homology of coding region was only 71%, that was, they were two completely different viruses. In particular, the inventors found that CVB1 had a significantly superior tumor killing effect, and had killing activities on most of the detected tumor cells at least tens of times, or even hundreds of times, as compared with CVB3, by comparing the killing efficacies of CVB1-WT and CVB3-WT on different types of tumor cells (Table 3). It can be seen from this that CVB1 of the present invention can produce more potent anti-tumor activity at a relatively lower dose, which could greatly improve the safety of administration while ensuring the therapeutic efficacy, and thus is particularly suitable for anti-tumor treatment.

TABLE 3
Comparison of results of in vitro anti-tumor tests of CVB1-WT and CVB3-WT

| | | | | | | | | | | Potency |
|---|---|---|---|---|---|---|---|---|---|---|
| | | CVB1-WT | | | | CVB3-WT | | | | multiple |
| | | MOI | | | EC50 | MOI | | | EC50 | (EC50 |
| Cell lines | | 1 | 0.1 | 0.01 | (MOI) | 1 | 0.1 | 0.01 | (MOI) | ratio) |
| Lung cancer | NCI-H1 | ++ | ++ | + | 0.02 | ++ | − | − | 0.97 | 48.5 |
| cell line | 975 | | | | | | | | | |
| | A549 | ++ | ++ | + | 0.02 | ++ | + | + | 0.82 | 41 |
| | EBC-1 | ++ | ++ | ++ | <0.01 | ++ | ++ | + | 0.08 | >8 |
| | NCI-H1 | ++ | ++ | + | 0.03 | ++ | + | + | 0.89 | 29.6 |
| | 703 | | | | | | | | | |
| Esophageal | TE-1 | ++ | ++ | + | 0.04 | + | − | − | 15.23 | 380.75 |
| cancer cell | | | | | | | | | | |
| line | | | | | | | | | | |
| Ovarian | CaOV3 | ++ | ++ | − | 0.03 | ++ | + | − | 0.78 | 26 |
| cancer | | | | | | | | | | |
| cell line | | | | | | | | | | |
| Endometrial | HEC-1- | ++ | ++ | + | 0.05 | + | − | − | 13.34 | 266.8 |
| cancer cell | A | | | | | | | | | |
| line | | | | | | | | | | |
| Pancreatic | Capan-2 | ++ | + | − | 0.08 | − | − | − | >10 | >125 |
| cancer | AsPC-1 | ++ | ++ | ++ | <0.01 | ++ | + | − | 0.96 | >96 |
| cell line | SW1990 | ++ | ++ | + | 0.02 | ++ | + | − | 0.82 | 41 |
| | CFPAC- | ++ | ++ | + | 0.03 | + | − | − | 25.71 | 857 |
| | 1 | | | | | | | | | |
| Tongue | CAL27 | ++ | ++ | + | 0.03 | + | − | − | 17.20 | 573.3 |
| cancer cell | SCC-25 | ++ | ++ | ++ | <0.01 | ++ | + | − | 0.78 | >78 |
| line | | | | | | | | | | |
| Renal | 786-O | ++ | + | − | 0.05 | + | − | − | 17.84 | 356.8 |
| cancer | | | | | | | | | | |
| cell line | | | | | | | | | | |
| Prostate | DU145 | ++ | ++ | ++ | <0.01 | ++ | ++ | + | 0.05 | >5 |
| cancer | | | | | | | | | | |
| cell line | | | | | | | | | | |
| Nasopharyngeal | CNE | ++ | ++ | + | 0.03 | ++ | + | − | 0.88 | 29.33 |
| cancer cell line | | | | | | | | | | |
| Bladder | 5637 | ++ | ++ | ++ | <0.01 | ++ | + | − | 0.53 | 53 |
| cancer | | | | | | | | | | |
| cell line | | | | | | | | | | |

Note:
“−” indicated that there was no significant difference in cell survival rate between virus treatment group and MOCK group;
“+” indicated that after virus treatment, the number of cells was reduced, the survival rate was greater than 50% but was significantly different from that of MOCK group;
“++” indicated that the cell survival rate after virus treatment was less than 50%, and was significantly different from that of the MOCK group; EC50, half of effective dose, referring to herein as the MOI of viruses by which the cell survival rate drops to 50%; potency multiple, referring to herein as the multiple of oncolytic efficacy of CVB1 and CVB3 on specific cells, that is, the EC50 ratio.

In addition, the results of in vitro anti-tumor experiments of CVB1-miR133&206T, CVB1-GM-CSF and CVB1-Anti-PD-1 showed that the above-mentioned modified forms of CVB1 all retained the killing effect of the parental strain of wild-type CVB1 on the detected tumor cells, and the significant killing effects of the parental strain of wild-type CVB1 on colorectal cancer cell lines, gastric cancer cell lines, lung cancer cell lines, liver cancer cell lines, cervical cancer cell lines, endometrial cancer cell lines, pancreatic cancer cell lines, prostate cancer cell lines, nasopharyngeal cancer cell lines, tongue cancer cell lines, laryngeal cancer cell lines, glioma cell lines, and neuroblastoma cell lines, in which the results of CCK-8 detection of the oncolytic activities to human colorectal cancer cell line SW480, human gastric cancer cell line AGS, human endometrial cancer cell line Ishikawa line and human glioma cell line GBM were shown in Table 4.

It is particularly worth noting that CVB1-HRV2 has significant killing activity to some tumor cells to which CVB1-WT showed weak killing activity, and brings a significant beneficial technical effect; in which the results of CCK-8 detection of the oncolytic activity to human thyroid cancer cell line SW579 were shown in Table 5.

TABLE 4
Results of in vitro anti-tumor tests of CVB1-
miR133&206T, CVB1-GM-CSF and CVB1-Anti-PD-1

MOI

| Cell lines | | 10 | 1 | 0.1 | 0.01 |
|---|---|---|---|---|---|
| CVB1-miR133&206T | SW480 | ++ | ++ | ++ | ++ |
| | AGS | ++ | ++ | ++ | ++ |
| | Ishikawa | ++ | ++ | ++ | ++ |
| | GBM | ++ | ++ | ++ | ++ |
| CVB1-GM-CSF | SW480 | ++ | ++ | ++ | ++ |
| | AGS | ++ | ++ | ++ | ++ |
| | Ishikawa | ++ | ++ | ++ | ++ |
| | GBM | ++ | ++ | ++ | ++ |
| CVB1-Anti-PD-1 | SW480 | ++ | ++ | ++ | ++ |
| | AGS | ++ | ++ | ++ | ++ |
| | Ishikawa | ++ | ++ | ++ | ++ |
| | GBM | ++ | ++ | ++ | ++ |

Note:
“−” indicated that there was no significant difference in cell survival rate between virus treatment group and MOCK group;
“+” indicated that after virus treatment, the number of cells was reduced, the survival rate was greater than 50% but was significantly different from that of MOCK group;
“++” indicated that the cell survival rate after virus treatment was less than 50%, and was significantly different from that of the MOCK group.

TABLE 5
Comparison of results of in vitro oncolytic tests of CVB1-WT
and CVB1-HRV2 on human thyroid cancer cell line SW579

MOI

| Cell lines | 10 | 1 | 0.1 | 0.01 |
|---|---|---|---|---|
| CVB1-WT | ++ | + | − | − |
| CVB1-HRV2 | ++ | ++ | ++ | + |

Note:
“−” indicated that there was no significant difference in cell survival rate between virus treatment group and MOCK group;
“+” indicated that after virus treatment, the number of cells was reduced, the survival rate was greater than 50% but was significantly different from that of MOCK group;
“++” indicated that the cell survival rate after virus treatment was less than 50%, and was significantly different from that of the MOCK group.

2.5 Serial Passaging of CVB1 for Adaptation

In this example, CVB1 was serially passaged for adaptation in a certain tumor cell to obtain a strain with enhanced killing activity on the tumor cell.

The wild-type enterovirus CVB1 was serially passaged for adaptation in human osteosarcoma cell line U2OS, human thyroid cancer cell line SW579 and human bladder cancer cell line J82, on which the oncolytic effect of CVB1 was not very significant. The specific methods were as follows:

One kind of the above tumor cells was evenly plated on a 10 cm cell culture plate, and the culture conditions were a corresponding cell culture medium containing 10% fetal bovine serum, glutamine, penicillin and streptomycin, 37° C., 5% CO₂, saturated humidity. When the cell confluence reached 90% or more, the cell culture medium was replaced with serum-free cell culture medium, each plate was inoculated with 10⁷ TCID50 of CVB1 virus, and the culture environment was changed to 33° C., 5% CO₂, saturated humidity. When CVB1 proliferated in tumor cells and caused CPE in the cells (after infection for up to 3 day), the cells and their culture supernatant were harvested. After freeze-thawing for three cycles, centrifugation was performed at 4000 rpm for 10 min at 4° C. The centrifugal supernatant was taken and added onto new tumor cells with a confluence of more than 90%, to complete one round of virus passage. The passage was repeated for more than 10 times in this way, and a part of the virus solution of each round of passage was taken out for titration of virus in RD cells, and the specific method referred to Example 2.3. Generally, the virus replication capacity would increase with the increase of generations, and when a relatively high infectious titer was reached and the virus replication was stable in the tumor cell, the adapted strain of CVB1 for the tumor cells was obtained.

Subsequently, the human tumor cells U2OS, SW579 or J82 were inoculated to 96-well plates at 10⁴ cells/well by the method of the in vitro anti-tumor experiment described in Example 2.4. After the cells adhered, the medium in each well was replaced with the corresponding cell culture medium free of serum, followed by incubation at 37° C. for 30 min, and then the serially passaged CVB1 strains adapted for each of the above kinds of cells were inoculated at MOIs of 10, 1, 0.1, and 0.01 (the viral titers were detected on RD cells), respectively. Subsequently, CPE of the cells were monitored daily by a microscope, and the cell survival rate was detected using CCK-8 method 72 hours after the infection and culture of viruses.

The results were shown in Table 6. After serial passaging of the wild-type CVB1 in a kind of tumor cells on which CVB1 had poor oncolytic effect, its killing activity on the tumor cell was significantly enhanced, indicating that the CVB1 adapted strain with enhanced oncolytic effect on the tumor cells could be obtained by the above-mentioned serial passaging method.

TABLE 6
Results of in vitro killing experiment of CVB1 on a tumor cell
after serial passaging for adaptation in the tumor cell

MOI

| Cell lines | 10 | 1 | 0.1 | 0.01 |
|---|---|---|---|---|
| U2OS | ++ | ++ | ++ | − |
| SW579 | ++ | ++ | + | + |
| J82 | ++ | ++ | ++ | + |

Note:
“−” indicated that there was no significant difference in cell survival rate between virus treatment group and MOCK group;
“+” indicated that after virus treatment, the number of cells was reduced, the survival rate was greater than 50% but was significantly different from that of MOCK group;
“++” indicated that the cell survival rate after virus treatment was less than 50%, and was significantly different from that of the MOCK group.

2.6 Evaluation of Oncolytic Effect of Genomic RNA of CVB1

In this example, a large amount of infectious live viruses of CVB1 could be produced by transfecting the purified genomic RNA of CVB1 into a certain kind of tumor cells, and thus kill the tumor cells.

The viral genomic RNA was first obtained by in vitro transcription, and this method could be found in, for example, Hadac E M, Kelly E J and Russell S J. Mol Ther, 2011, 19(6): 1041-1047. Specifically, the infectious cloning plasmid of wild-type CVB1 obtained in Example 1 was linearized, and the linearized plasmid was used as a template for in vitro transcription using MEGAscript™ T7 Transcription Kit (Thermo Fisher Scientific, AM1333) so as to produce a large amount of viral RNA. And the obtained viral RNA was purified using MEGAclear™ Transcription Clean-Up Kit (Thermo Fisher Scientific, AM1908) for next use. The RNA electropherogram of one sample was shown in FIG. 2.

Subsequently, according to the method of the in vitro anti-tumor experiment described in Example 2.4, the human cervical cancer tumor cell line Hela was inoculated to a 24-well plate at 10⁵ cells/well. After the cells adhered, the medium in each well was replaced with a corresponding cell culture medium free of serum, followed by incubation at 37° C. for 30 min. Then Hela cells were transfected with purified virus RNA at 1 μg per well using transfection reagent Lipofectamine® 2000 (Thermo Fisher Scientific, 11668019), and the negative control group was transfected with irrelevant RNA nucleic acid molecules. Subsequently, CPE of the cells were monitored daily by a microscope.

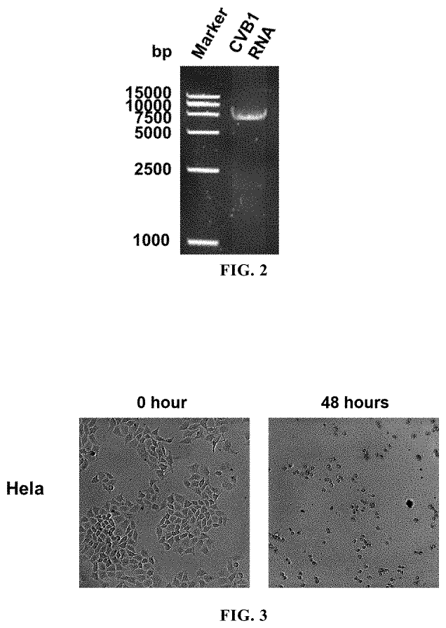

The results showed that CPE began to appear in the Hela cells transfected with genomic RNA of CVB1 about 8 hours after transfection, and then the cytopathy gradually increased. After 48 hours, the survival rate was measured using the CCK8 method, the Hela cells had almost all died and lysed. And the micrographs of Hela cells at 0 and 48 hours after infection were shown in FIG. 3. The culture supernatant was inoculated into new Hela cells and CPE was quickly produced. The results indicated that the direct administration with the nucleic acid of CVB1 also had good killing activity and could be used to treat tumors.

### Example 3: In Vivo Anti-Tumor Experiment of CVB1 and Modified Forms Thereof

3.1 Viruses, Cell Lines and Laboratory Animals

(1) Viruses: In this example, the CVB1-WT (SEQ ID NO: 12), CVB1-HRV2 (SEQ ID NO: 13), CVB1-miR133&206T (SEQ ID NO: 14), CVB1-GM-CSF (SEQ ID NO: 15) and CVB1-Anti-PD-1 (SEQ ID NO: 16) provided in Example 1 were used. For the virus culture and virus titer determination methods, see Examples 2.2 and 2.3, respectively.

(2) Cell lines: human breast cancer cell line BcaP37 (CCTCC deposit number: GDC206), human non-small cell lung cancer cell lines A549 (ATCC® Number: CCL-185™) and SPC-A-1 (CCTCC deposit number: GDC050), human Burkitt's lymphoma cell line Raji (ATCC® Number: CCL-86™), human endometrial cancer cell lines Ishikawa (ECACC No. 99040201) and HEC-1-B (ATCC® Number: HTB-113™), human cervical cancer cell lines Hela (ATCC® Number: CCL-2™) and C-33A (ATCC® Number: HTB-31™), and human glioma cell line GBM (primary tumor cell line isolated from patient tumor tissue). Except that Raji was cultured using RPMI-1640 medium and C-33A was cultured using MEM medium, the above cells were cultured using DMEM medium, and the above mediums were added with 10% fetal bovine serum, glutamine and penicillin-streptomycin. All the above cells were cultured under standard conditions of 37° C. and 5% CO₂.

(3) Laboratory animals: 6-8-week-old female C.B17 SCID mice were from Shanghai Silaike Experimental Animal Co., Ltd.; according to the protocol approved by Experimental Animal Center and Ethics Committee, Xiamen University, the mice were raised under SPF conditions.

3.2 In Vivo Anti-Tumor Experiments of Viruses

The tumor cells used for subcutaneous tumor formation in SCID mice were digested with 0.01% trypsin, and then resuspended into a single cell suspension using cell culture medium containing 10% fetal bovine serum. The cell density of the suspension was counted. The cells were precipitated by centrifugation under 1000 g for 3 min, and then the cells were resuspended with an appropriate volume of PBS to reach a concentration of about 10⁶-10⁷ cells/100 μl PBS. The tumor cells were subcutaneously inoculated in the back of SCID mice at 10⁶-10⁷ cells/100 μl PBS/site with a syringe. When the tumor cells formed a tumor mass of approximately 100 mm³ under the skin of SCID mice after about 14-21 days, the tumor-bearing SCID mice were randomly divided into experimental groups (administrated with CVB1-WT, CVB1-HRV2, CVB1-miR133&206T, CVB1-GM-CSF or CVB1-Anti-PD-1) and negative control group, with 4 animals per group (n=4). Oncolytic virus (CVB1-WT, CVB1-HRV2, CVB1-miR133&206T, CVB1-GM-CSF or CVB1-Anti-PD-1) at 10⁶ TCID50/100 μl serum-free medium/tumor mass or equivalent amount of serum-free medium were intratumorally injected every two days, for a total of 5 treatments. The tumor size was measured with a vernier caliper and recorded every two days, and the method for calculating the tumor size was:

Tumor size (mm³)=tumor length value×(tumor width value)/2.

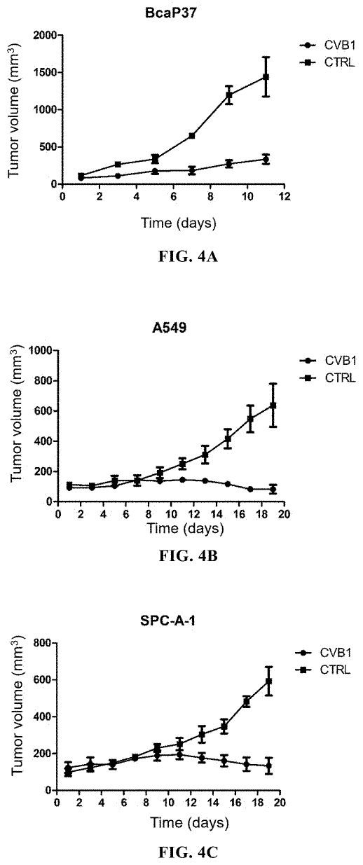

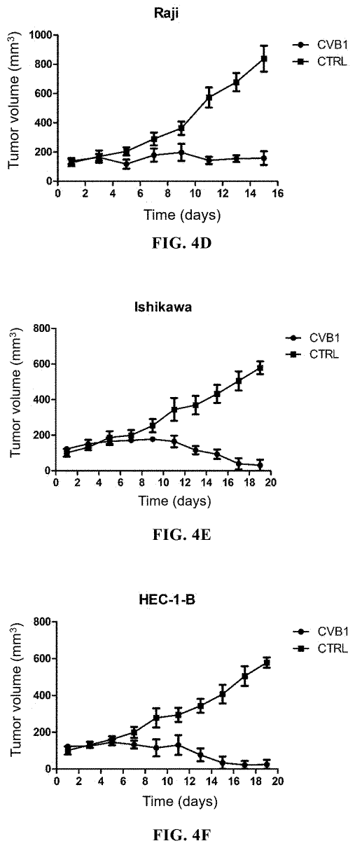

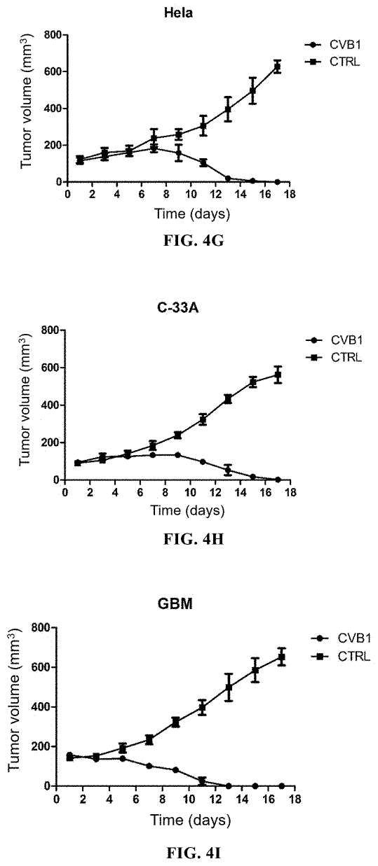

The treatment results of CVB1-WT on the above six tumors were shown in FIGS. 4A to 41, respectively. The results showed that after the challenge of CVB1-WT, the growth of the detected tumors of BcaP37 (A), A549 (B), SPC-A-1 (C), Raji (D), Ishikawa (E), HEC-1-B (F), Hela (G), C-33A (H) and GBM (I) gradually slowed down and arrested, and the tumors were even lysed and disappeared; in contrast, the tumors in the negative group (CTRL) maintained normal growth, and the tumor sizes were significantly larger than those in the experimental groups.

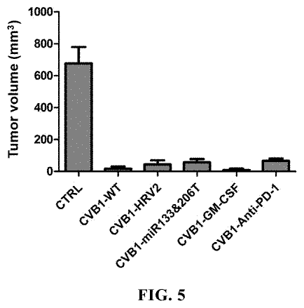

FIG. 5 showed the results obtained after a treatment of the GBM tumor model with CVB1-WT, CVB1-HRV2, CVB1-miR133&206T, CVB1-GM-CSF, or CVB1-Anti-PD-1 for 10 days. The results showed that, as compared with the negative control group without oncolytic virus treatment, the tumors were significantly reduced in volume and even almost disappeared after being treated with CVB1-WT, CVB1-HRV2, CVB1-miR133&206T, CVB1-GM-CSF and CVB1-Anti-PD-1 respectively, and the reduction extents in tumor volume after treatment with the five oncolytic viruses were similar. The above results indicated that CVB1-WT, CVB1-HRV2, CVB1-miR133&206T, CVB1-GM-CSF and CVB1-Anti-PD-1 all exhibited remarkable and favorable anti-tumor activity in vivo.

Although the specific embodiments of the present invention have been described in detail, those skilled in the art will understand that various modifications and changes can be made to the details based on all the teachings that have been published, and these changes are within the scope of the present invention. The entirety of the invention is given by the appended claims and any equivalents thereof

What is claimed is:

1. Use of a Coxsackievirus B1 (CVB1) or a modified form thereof or a nucleic acid molecule for treating a tumor in a subject, or for manufacture of a medicament for treating a tumor in a subject; wherein the nucleic acid molecule comprises a sequence selected from the following:
(1) a genomic sequence or cDNA sequence of the CVB1 or modified form thereof; and
(2) a complementary sequence of the genomic sequence or cDNA sequence.

2. Use according to claim 1, wherein the CVB1 is a wild-type CVB1;
preferably, the CVB1 is a clinical isolate isolated from an individual infected with Coxsackievirus B1;
preferably, the genomic sequence of the CVB1 or modified form thereof has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% to a nucleotide sequence as shown in SEQ ID NO: 12; more preferably, the genomic sequence of the CVB1 or modified form thereof is a nucleotide sequence as shown in SEQ ID NO: 12;
preferably, the cDNA sequence of the CVB1 or modified form thereof has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% to a nucleotide sequence as shown in SEQ ID NO: 1; more preferably, the cDNA sequence of the CVB1 or modified form thereof is a nucleotide sequence as shown in SEQ ID NO: 1.

3. Use according to claim 1 or 2, wherein the modified form is a modified CVB1 which has a substitution, insertion or deletion of one or more nucleotides in its genome as compared to a wild-type CVB1;
preferably, compared to the wild-type CVB1, the modified CVB1 has one or more modifications selected from the following:
(1) one or more mutations in an untranslated region (e.g. 5′UTR or 3′UTR);
(2) an insertion of one or more exogenous nucleic acids;
(3) a deletion or mutation of one or more endogenous genes; and
(4) any combination of the above three items.

4. Use according to claim 3, wherein the modified CVB1 comprises one or more mutations in a 5′ untranslated region (5′ UTR);
preferably, the modified CVB1 has a substitution of all or part of the 5′UTR sequence;
preferably, an internal ribosome entry site (IRES) sequence in the 5′UTR of the modified CVB1 is replaced with an exogenous IRES sequence, such as an internal ribosome entry site sequence of human rhinovirus 2 (HRV2);
preferably, the internal ribosome entry site sequence of human rhinovirus 2 (HRV2) is shown in SEQ ID NO: 2.

5. Use according to claim 3 or 4, wherein the modified CVB1 comprises an exogenous nucleic acid;
preferably, the exogenous nucleic acid encodes a cytokine (e.g., GM-CSF, preferably human GM-CSF), or an anti-tumor protein or polypeptide (e.g., scFV against PD-1 or PD-L1, preferably scFv against human PD-1 or PD-L1);
preferably, the exogenous nucleic acid is inserted between 5′UTR and VP4 gene, or between VP1 gene and 2A gene of a genome of the modified CVB1;
preferably, the exogenous nucleic acid comprises a target sequence of one or more (e.g., 2, 3, or 4) microRNA;
preferably, the target sequence of microRNA is inserted in a 3′ untranslated region (3′UTR) of a genome of the modified CVB1;
preferably, the exogenous nucleic acid comprises a target sequence of miR-133 and/or miR-206;
preferably, the target sequence of miR-133 is shown in SEQ ID NO: 3;
preferably, the target sequence of miR-206 is shown in SEQ ID NO:4.

6. Use according to any one of claims 3 to 5, wherein the modified CVB1 comprises at least one insertion of an exogenous nucleic acid as defined in claim 5 and/or at least one mutation in the untranslated region as defined in claim 4.

7. Use according to any one of claims 3 to 6, wherein the modified CVB1 has one of the following characteristics:
(1) the genomic sequence of the modified CVB1 has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% to a nucleotide sequence selected from the following: the nucleotide sequences as shown in SEQ ID NOs: 13-16; preferably the genomic sequence of the modified CVB1 is any one selected from the nucleotide sequences as shown in SEQ ID NOs: 13-16;
2) the cDNA sequence of the modified CVB1 has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% to a nucleotide sequence selected from the following: the nucleotide sequences as shown in SEQ ID NOs: 8-11; preferably, the cDNA sequence of the modified CVB1 is any one selected from the nucleotide sequences as shown in SEQ ID NOs: 8-11.

8. Use according to any one of claims 1 to 7, wherein the medicament comprises one or several of the CVB1 and modified form thereof.

9. Use according to any one of claims 1 to 8, wherein the nucleic acid molecule consists of the genomic sequence or cDNA sequence of the CVB1 or modified form thereof, or a complementary sequence of the genomic sequence or cDNA sequence;
preferably, the nucleic acid molecule has the genomic sequence of the CVB1 or modified form thereof;
preferably, the nucleic acid molecule has a nucleotide sequence as shown in any one of SEQ ID NOs: 12-16.

10. Use according to any one of claims 1 to 8, wherein the nucleic acid molecule is a vector (e.g., a cloning vector or expression vector) comprising a genomic sequence or cDNA sequence of the CVB1 or modified form thereof, or a complementary sequence of the genomic sequence or cDNA sequence;
preferably, the nucleic acid molecule is a vector (e.g., a cloning vector or expression vector) comprising the cDNA sequence of the CVB1 or modified form thereof, or the complementary sequence of the cDNA sequence;
preferably, the nucleic acid molecule is a vector comprising a nucleotide sequence as shown in any one of SEQ ID NOs: 1, 8-11, or a complementary sequence thereof.

11. Use according to any one of claims 1 to 10, wherein the medicament further comprises an additional pharmaceutically active agent having anti-tumor activity, such as an additional oncolytic virus, chemotherapeutic agent or immunotherapeutic agent;
preferably, the additional oncolytic virus is selected from the group consisting of herpes virus, adenovirus, parvovirus, reovirus, Newcastle disease virus, vesicular stomatitis virus, measles virus or any combination thereof,
preferably, the chemotherapeutic agent is selected from the group consisting of 5-fluorouracil, mitomycin, methotrexate, hydroxyurea, cyclophosphamide, dacarbazine, mitoxantrone, anthracyclines (e.g., epirubicin or doxorubicin), etoposide, platinum compounds (e.g., carboplatin or cisplatin), taxanes (e.g., paclitaxel or docetaxel), or any combination thereof;
preferably, the immunotherapeutic agent is selected from the group consisting of immune checkpoint inhibitors (e.g., PD-L1/PD-1 inhibitors or CTLA-4 inhibitors), tumor-specific targeting antibodies (e.g., rituximab or herceptin), or any combination thereof.

12. Use according to any one of claims 1 to 11, which has at least one of the following characteristics:
(1) the tumor is selected from the group consisting of colorectal cancer, gastric cancer, lung cancer, liver cancer, ovarian cancer, endometrial cancer, cervical cancer, melanoma, breast cancer, kidney cancer, pancreatic cancer, lymphoma, osteogenic sarcoma, prostate cancer, glioma, neuroblastoma, tongue cancer, nasopharyngeal cancer, squamous cell carcinoma of nasal septum, pharyngeal squamous cell carcinoma, squamous cell carcinoma of submandibular gland, laryngeal cancer, thyroid cancer, thyroid ductal carcinoma and bladder cancer;
(2) the subject is a mammal, such as a human.

13. A method for treating a tumor, comprising a step of administering to a subject in need thereof an effective amount of a CVB1 or a modified form thereof, or an effective amount of a nucleic acid molecule; wherein the nucleic acid molecule comprises a sequence selected from the following:
(1) a genomic sequence or cDNA sequence of the CVB1 or modified form thereof; and
(2) a complementary sequence of the genomic sequence or cDNA sequence;
preferably, the CVB1 or modified form thereof, or the nucleic acid molecule is defined as in any one of claims 1 to 12;
preferably, one or more of the CVB1 and modified form thereof is administered to the subject;
preferably, the tumor is selected from the group consisting of colorectal cancer, gastric cancer, lung cancer, liver cancer, ovarian cancer, endometrial cancer, cervical cancer, melanoma, breast cancer, kidney cancer, pancreatic cancer, lymphoma, osteogenic sarcoma, prostate cancer, glioma, neuroblastoma, tongue cancer, nasopharyngeal cancer, squamous cell carcinoma of nasal septum, pharyngeal squamous cell carcinoma, squamous cell carcinoma of submandibular gland, laryngeal cancer, thyroid cancer, thyroid ductal carcinoma and bladder cancer;
preferably, the subject is a mammal, such as a human.

14. A pharmaceutical composition, comprising a CVB1 or a modified form thereof, or a nucleic acid molecule; and, a pharmaceutically acceptable carrier or excipient; wherein the nucleic acid molecule comprises a sequence selected from the following:
(1) a genomic sequence or cDNA sequence of the CVB1 or modified form thereof; and
(2) a complementary sequence of the genomic sequence or cDNA sequence;
preferably, the CVB1 or modified form thereof, or the nucleic acid molecule is defined as in any one of claims 1 to 12;
preferably, the pharmaceutical composition further comprises an additional pharmaceutically active agent having anti-tumor activity, such as an additional oncolytic virus, chemotherapeutic agent or immunotherapeutic agent;
preferably, the pharmaceutical composition is used for treating a tumor in a subject;
preferably, the tumor is selected from the group consisting of colorectal cancer, gastric cancer, lung cancer, liver cancer, ovarian cancer, endometrial cancer, cervical cancer, melanoma, breast cancer, kidney cancer, pancreatic cancer, lymphoma, osteogenic sarcoma, prostate cancer, glioma, neuroblastoma, tongue cancer, nasopharyngeal cancer, squamous cell carcinoma of nasal septum, pharyngeal squamous cell carcinoma, squamous cell carcinoma of submandibular gland, laryngeal cancer, thyroid cancer, thyroid ductal carcinoma and bladder cancer;
preferably, the subject is a mammal, such as a human.

15. A modified CVB1, which has a substitution of an internal ribosome entry site (IRES) sequence in a 5′UTR with an internal ribosome entry site sequence of human rhinovirus 2 (HRV2) as compared to a wild-type CVB1.

16. The modified CVB1 according to claim 15, wherein the modified CVB1 has at least one of the following characteristics:
1) the internal ribosome entry site sequence of human rhinovirus 2 (HRV2) is shown in SEQ ID NO: 2;
2) the wild-type CVB1 has a genomic sequence with a sequence identity of at least 70%, at least 80%, at least 85%, at least 90%, at least 91%, at least 92%, at least 93%, at least 94%, at least 95%, at least 96%, at least 97%, at least 98%, at least 99%, or 100% to a nucleotide sequence as shown in SEQ ID NO: 12; preferably, the genomic sequence of the wild-type CVB1 is a nucleotide sequence as shown in SEQ ID NO: 12;
3) the wild type CVB1 has a cDNA sequence with a sequence identity of at least 70%, at least 80%, at least 85%, at least 90%, at least 91%, at least 92%, at least 93%, at least 94%, at least 95%, at least 96%, at least 97%, at least 98%, at least 99%, or 100% to a nucleotide sequence as shown in SEQ ID NO: 1; preferably, the cDNA sequence of the wild-type CVB1 is a nucleotide sequence as shown in SEQ ID NO: 1;

17. The modified CVB1 according to claim 15 or 16, wherein the modified CVB1 further comprises an exogenous nucleic acid;
preferably, the exogenous nucleic acid encodes a cytokine (e.g., GM-CSF, preferably human GM-CSF), or an anti-tumor protein or polypeptide (e.g., scFv against PD-1 or PD-L1, preferably scFv against human PD-1 or PD-L1);
preferably, the exogenous nucleic acid is inserted between 5′UTR and VP4 gene, or between VP1 gene and 2A gene of a genome of the modified CVB1;
preferably, the exogenous nucleic acid comprises a target sequence of one or more (e.g., 2, 3 or 4) microRNA;
preferably, the target sequence of microRNA is inserted in a 3′ untranslated region (3′UTR) of a genome of the modified CVB1;
preferably, the exogenous nucleic acid comprises a target sequence of miR-133 and/or miR-206;
preferably, the target sequence of miR-133 is shown in SEQ ID NO: 3;
preferably, the target sequence of miR-206 is shown in SEQ ID NO:4.

18. The modified CVB1 according to any one of claims 15 to 17, wherein the modified CVB1 has one of the following characteristics:
1) the modified CVB1 has a genomic sequence with a sequence identity of at least 70%, at least 80%, at least 85%, at least 90%, at least 91%, at least 92%, at least 93%, at least 94%, at least 95%, at least 96%, at least 97%, at least 98%, at least 99%, or 100% to a nucleotide sequence as shown in SEQ ID NO: 13; preferably the genomic sequence of the modified CVB1 is a nucleotide sequence as shown in SEQ ID NO: 13;
2) the modified CVB1 has a cDNA sequence with a sequence identity of at least 70%, at least 80%, at least 85%, at least 90%, at least 91%, at least 92%, at least 93%, at least 94%, at least 95%, at least 96%, at least 97%, at least 98%, at least 99%, or 100% to a nucleotide sequence as shown in SEQ ID NO: 8; preferably the cDNA sequence of the modified CVB1 is a nucleotide sequence as shown in SEQ ID NO: 8.

19. A nucleic acid molecule, comprising a sequence selected from the following:
(1) a genomic sequence or cDNA sequence of the modified CVB1 according to any one of claims 15 to 18; and
(2) a complementary sequence of the genomic sequence or cDNA sequence.

20. The nucleic acid molecule according to claim 19, wherein the nucleic acid molecule consists of the genomic sequence or cDNA sequence of the modified CVB1, or the complementary sequence of the genomic sequence or cDNA sequence;
preferably, the nucleic acid molecule has the genomic sequence of the modified CVB1;
preferably, the nucleic acid molecule has a nucleotide sequence as shown in SEQ ID NO: 13.

21. The nucleic acid molecule according to claim 19, wherein the nucleic acid molecule is a vector (e.g., a cloning vector or expression vector) comprising the genomic sequence or cDNA sequence of the modified CVB1, or the complement of the genomic sequence or cDNA sequence;
preferably, the nucleic acid molecule is a vector (e.g., a cloning vector or an expression vector) comprising the cDNA sequence of the modified CVB1, or the complementary sequence of the cDNA sequence;
preferably, the nucleic acid molecule is a vector comprising a nucleotide sequence as shown in SEQ ID NO: 8 or complementary sequence thereof.


SEQUENCE LISTING (20 sequences)

SEQ ID NO: 1 - DNA, 7371 bp, Artificial Sequence
  cDNA sequence of CVB1-WT
tcctgtgggttgatcccacccacagggcccactgggcgctagcacactggtattccggtacctttgtgcgcctgttttatacacccttcccaagtgtaacttagaagcttatcacacacggtcaacaggtgactcagtatgccaactgggtcttgatcaagcacttctgtttccccggactgagtatcaataagctgctcacgcggctgaaggagaaaacgttcgttaaccggccaattacttcgagaaacctagtaacaccatggaggttgcgcagtgtttcgctccacacaaccccagtgtagatcaggccgatgagtcaccgcactcctcacgggcgaccgtggcggtggctgcgctggcggcctgcccatgggataacccatgggacgcttcaatactgacatggtgcgaagagtctattgagctaattggtagtcctccggcccctgaatgcggctaatcctaactgcggagcagatacccacacgccagtgggcagtctgtcgtaatgggcaactctgcagcggaaccgactactttgggtgtccgtgtttccttttatttttatactggctgcttatggtgacaattgagagattgttaccatatagctattggattggccatccagtgacaaacagagcgattatttatttatttgttggttatatcccattaagccaaaaggccctagttactctcaactttatactattgcttaatacaacgaaatgggagctcaggtgtctacacaaaagacgggtgcacatgagactggcttgaatgccagcggcaattctgtcatccattacaccaacatcaattactacaaggatgctgcatccaactccgcaaataggcaagacttcacacaagacccaggtaagttcactgaacctgtaaaggacatcatggtgaaaactatgcctgcgttgaactcaccctctgctgaggaatgcggctacagtgacagagtgcgttccatcacattaggtaattccactatcaccacccaagagtgtgccaacgtggtcgtcgggtacggagtgtggccagagtatctaaaagacaacgaggccacagccgaagatcagcccacacaacctgatgtggccacctgccgcttctatactttagaatcagtgcagtggatgaaaaattcggccgggtggtggtggaagctgcctgatgcactgtcacagatgggcttgtttggtcaaaacatgcagtaccactacttgggaagaacagggtatactatacacgtacaatgcaatgcctcaaagttccatcaaggatgcctgcttgtagtgtgtgtgcctgaagctgagatggggtgctctaacttgaacaacacgcccgaattcagtgagttatccggaggagacagtgccagaatgttcaccgatacacaggtcggggagtcaaacgccaagaaagtacagacagcggtgtggaacgccggaatgggcgttggagtgggtaatctcactattttccctcaccaatggatcaacctgaggaccaacaacagtgcaacgctagtgatgccttacataaacagcgtccccatggataacatgttcaggcacaataacttaacactaatgattatcccatttgtaccgcttaattacagcgagggatcctcaccgtacgtgcccataacagtcaccatcgctcctatgtgtgcagagtacaacgggctacggctggccagcaaccagggtctaccagttatgacaacacctgggagcacgcaatttctaacttcagatgacttccagtcaccgtcagcgatgccacagtttgatgtcaccccagagatgcaaataccaggccgcgtaaataatttgatggagattgccgaggtggactcagtggtgcctgtgaataacacagaggacaacgtgagtagccttaaggcataccagattccagtacagtccaacagtgacaatggtaagcaggtttttggttttcccttacaaccgggtgccaacaacgttctgaatagaaccctcttgggtgaaattctaaactattacacccattggagtggcagcataaaacttacattcatgttctgtgggtctgccatggccactggcaagttccttctggcatactcaccacctggagcaggagtcccgaaaaaccgaaaagatgctatgctgggcacccacgtcatatgggatgttgggttacaatcaagctgcgtgttgtgcgtaccgtggattagccaaacacactacagatacgtggttgaggatgaatacacagcagccggatatgttacatgctggtatcaaacaaatatcgtggtgccagctgatgtgcaaagctcatgtgacatcttgtgctttgtttcagcctgcaacgatttctctgtgcggatgctgaaagatacgccctttataagacaggacacattttatcacggcccagtggaggagtctgtggatcgtgcagtggcacgtgtggcggacacgattagttcacggcccactaactcggagtcaattccagcgctcaccgccgccgagactgggcatacctcccaagttgtgcctagcgacaccatgcaaacaagacatgtgaaaaactaccactcacggtccgaatcgtccattgaaaatttcctatgccggtctgcctgtgtatactatgccacatacaccaataactcaaaaaaagaggggtttgcagagtgggttatcaacactagacaggtggcacagctaagaagaaagttggagctcttcacatatctaaggtttgacttagaactaacatttgttataacgagcgcacaacaacccagtaccgcctccagtgtagacgctcccgtgcaaacccatcagattatgtatgtgcctccaggtggccctgtaccaacaaaggttaaagattatgcatggcaaacttccacaaaccccagcgttttctggacagaaggaaacgccccaccaaggatgtccatcccattcattagcattggtaatgcgtatagttgtttttatgatgggtggacgcagttttcaagaaacggggtctatggcatcaataccctcaacaacatgggcacgttgtatatgaggcatgtcaatgaagcggggcagggtccaatcaaaagtactgttagaatatatttcaaacccaagcacgtgaaggcgtgggtgccacgtccgccaagattgtgtcaatatgagaagcagaaaaatgtcaattttagccctataggagtaaccacaagtagaacggacattataacaacaggcacttttggtcagcagtccggagcgatatatgtgggcaattacaggatagtcaataggcacctggcaacacactttgactggcaaaattgcatatgggaggattacaacagggacctcctagtatgcactacaacggcgcacggttgtgacaatattgcaagatgccagtgcacagcaggtgtgtattattgtgcttctaggaacaagcattacccggtgcattttgaaggaccaggcttggtggaggtccaggaaagtgaatattatccaaaaaggtatcaatcacacgtgcttcttgccgcagggttttctgagccgggggattgtggtggtattctcaggtgtgaacatggtgttataggcatcgtgaccatgggtggtgagggtgttgtcggctttgccgatgtgcgcgatctcttatggctggaagacgacgcaatggaacagggagtcagggactacgtggaacagcttggaaacgcctttggttctgggtttaccaaccagatttgcgagcaagtcaatctgttaaaagagtcactaataggccaagactccatcttggaaaaatcactcaaggcattagtgaaaatcatatcagcactggttatcgtggtgagaaaccacgatgacctcataacagtcactgccaccttagccctgattggctgtaccacttcaccgtggcggtggctaaagaaaaaagtgactcaatactatgggattcccatggccgagagacagaacaatggctggctcaagaaattcacagaaatgaccaatgcatgcaagggcatggaatggatcgccatcaaaatccaaaagttcattgagtggctcaaaattaagatcttgccagaagtaaaggaaaaacacgagtttcttaccagacttaagcaactcccactcttagaaagccaaattgccaccatcgagcagagtgcaccatcacagagtgatcaggaacaactgttctccaatatacagtacttcgcgcactattgcagaaagtacgccccactgtacgcagctgaggcaaaaagagtgttctctctagaaaagaaaatgagcaactacatacagttcaagtccaaatgccgtattgaaccagtctgtttattgctccatggtagccctggagccggaaaatccgtggctaccaatttgattggacgctcacttgcagagaagctcaacagttcagtgtattcattgccaccagacccagaccattttgacggctataaacaacaagctgtcgtaatcatggatgacctgtgccagaacccagatgggaaagacgtgtccttgttctgccagatggtttcaagtgtagactttgttccaccaatggcagcacttgaggagaaaggcatactcttcacatcgcctttcgtgctcgcctctaccaacgcaggttccatcaacgctcccactgtgtcagacagcagagcccttgctagaaggttccacttcgacttgaacattgaggtcatctcaatgtacagccagaatggcaaaatcaacatgccaatgtcagtgaaggcttgtgatgatgagtgttgtccagttaatttcaagaggtgttgtccattagtgtgcggtaaggccattcagttcattgacagaagaacacaaatgaggtattcactagacatgcttgtaactgaaatgttcagggagtacaaccacagacatagtgtgggggcaacactcgaggcactcttccaaggcccacccgtgtacaaagagatcaagatcagtgttgcgccagaaacccctcccccaccggcaatagcagatctgctgaaatctgtggacagtgaagcagttagagagtactgcaaggaaaagggatggttggtgcctgagatcaattccactctacaaattgaaaagcacgtgagcagggccttcatttgcctgcaagcgctcaccacgtttgtttccgtggcggggataatttacatcatatacaaattgtttgctggcttccagggggcctacaccggtatgccaaaccaaaaacctaaagtaccaactttgaggcaggctaaggtgcagggaccggcattcgaattcgctgtggcaatgatgaaaagaaatgctagcacagtgaagaccgagtacggtgagttcacaatgcttggaatctatgacaggtgggcagtgctacctcgtcacgctagaccagggcctaccatcctaatgaatgaccaggaagtgggtgtggtggatgccaaggagttagtggacaaggatggcaccaatttagaattaacactcttgaagctcaatagaaatgagaaattcagagacatccgaggcttcttaacacgtgaggaggccgaagtcaacgaggctgtgcttgctattaacaccagcaagttcccaaatatgtacattccagttgggcaagtaacagattatggcttcctcaacttgggtggcacaccaactaagcgcatgctgatgtacaacttcccaacacgtgcaggccaatgtgggggagttctaatgtctacaggtaaagtgctcggaatacacgtcggtggtaatggacaccaagggttttcggcagccctgttacgccactatttcaatgacgagcagggagagattgagttcatcgagagctctaaggatgcaggctttccagtcattaacacacctagcaaaacaaagctcgagcccagtgttttccatcacgtgtttgagggaaacaaagaaccagcagtgttgaggaatggagacccaagactgaaagccaattttgaggaagccattttctccaagtacatcgggaatgtgaacacacatgtagacgagtacatgatggaagccgtcgaccattatgcaggacagttagctactttagatatcagtacagaacctatgaagcttgaggatgctgtctacggtactgagggcttggaagctctggacttgaccactagtgctgggtatccctatgtcgcccttggtataaaaaagagagacatcttatccaaaaagtctaaggacctgtctaaactgagagagtgtatggacaagtatgggttaaacctccctatggtcacctatgtgaaggatgaattgaggtcagcagagaaagtggctaagggcaaatccagactaatcgaggcgtccagtttaaatgactcagtggcaatgagacagactttcggtaacctgtataagacattccatctgaacccgggcatagtgactggcagtgcagttggatgcgatcccgacttgttctggagcaagattccagtcatgctcgatggtcacctcatcgcctttgactacagtggatatgatgccagcctgagtccggtgtggtttgcttgcttgaaactgttgttggaaaagctggggtactcacacaaggaaactaattacatagattacttgtgcaactcccaccatttgtacagagacaagcactactttgtgaggggaggaatgccatccgggtgttctggaaccagcattttcaattcaatgattaataacataatcatcagaacactgatgttgaaggtgtacaaagggatagatctagaccaatttaggatgattgcatatggtgacgacgtcattgcttcgtacccgtatccaattgatgcgtcattgctggctgaggctggcaaggggtatggtctgattatgacaccagcagacaaaggagagtgtttcaatgaagtaacgtggaccaacgtcactttcctgaaaagatacttcagagcggatgagcaatatcccttcctcgttcacccagtgatgccaatgaaggacattcatgagtcaattaggtggactaaagaccccaaaaacacccaagatcacgtgcgatccttgtgcttattggcctggcacaatggagagcaagaatatgagaattttgtatccaaaatcagaagcgtcccagttgggcgctgcttgacattgcctgcgttctcgactctgcgcaggaagtggttggattccttctaaaattgaagtacaatgtgatttaactcaattggcttaaccctaccacacttaccgaactagataacggtgtggtaggggtaaattc

SEQ ID NO: 2 - RNA, 507 nt, Artificial Sequence
  RNA sequence of internal ribosome entry site sequence of HRV2
aacuuagaaguuuuucacaaagaccaauagccgguaaucagccagauuacugaaggucaagcacuucuguuuccccggucaauguugauaugcuccaacagggcaaaaacaacugcgaucguuaaccgcaaagcgccuacgcaaagcuuaguagcaucuuugaaaucguuuggcuggucgauccgccauuuccccugguagaccuggcagaugaggcuagaaauaccccacuggcgacaguguucuagccugcguggcugccugcacacccuaugggugugaagccaaacaauggacaaggugugaagagccccgugugcucgcuuugaguccuccggccccugaauguggcuaaccuuaacccugcagcuagagcacguaacccaauguguaucuagucguaaugagcaauugcgggaugggaccaacuacuuuggguguccguguuucacuuuuuccuuuauauuugcuuauggugacaauauauacaauauauauauuggcacc

SEQ ID NO: 3 - RNA, 22 nt, Artificial Sequence
  RNA sequence of miR-133 target sequence
acagcugguugaaggggaccaa

SEQ ID NO: 4 - RNA, 22 nt, Artificial Sequence
  RNA sequence of miR-206 target sequence
ccacacacuuccuuacauucca

SEQ ID NO: 5 - RNA, 102 nt, Artificial Sequence
  RNA sequence of tandem sequence of miR-133 target sequence and miR-206 target sequence
acagcugguugaaggggaccaacgauacagcugguugaaggggaccaaaccgguccacacacuuccuuacauuccaucacccacacacuuccuuacauucca

SEQ ID NO: 6 - DNA, 435 bp, Artificial Sequence
  DNA sequence of GM-CSF gene
atgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtga

SEQ ID NO: 7 - DNA, 795 bp, Artificial Sequence
  DNA sequence of Anti-PD-1 scFv
atgaagcacctgtggttcttcctgctgctggtggccgctcctaggtgggtgctgtcccaggtgcagctggtgcagagcggcgtggaggtgaagaagcccggcgcttccgtgaaggtgtcctgcaaggcctccggctacaccttcaccaactactacatgtactgggtgaggcaggcccctggacagggactggagtggatgggcggcatcaacccttccaacggcggcaccaacttcaacgagaagttcaagaaccgggtgaccctgaccaccgactcctccaccaccaccgcctacatggagctgaagtccctgcagtttgacgacaccgccgtgtactactgcgccaggagggactaccggttcgacatgggcttcgactactggggccagggcacaaccgtgaccgtgtccagcggaggtggcggatctggagggggtggtagcggtggaggcgggagtgagatcgtgctgacccagtcccctgctacactgtccctgtcccccggcgagagggctacactgagctgcagggcctccaagggcgtgtccacctccggctactcctacctgcactggtaccagcagaagcctggacaggctcccaggctgctgatctacctggcctcctacctggagtccggcgtgcctgctaggttttccggcagcggcagcggcaccgatttcaccctgaccatctcctccctggagcccgaggacttcgccgtgtactactgccagcactccagggatctgcctctgaccttcggcggcggcaccaaggtggagatcaag

SEQ ID NO: 8 - DNA, 7251 bp, Artificial Sequence
  cDNA sequence of CVB1-HRV2
tcctgtgggttgatcccacccacagggcccactgggcgctagcacactggtattccggtacctttgtgcgcctgttttatacacccttcccaagtgtaacttagaagaacttagaagtttttcacaaagaccaatagccggtaatcagccagattactgaaggtcaagcacttctgtttccccggtcaatgttgatatgctccaacagggcaaaaacaactgcgatcgttaaccgcaaagcgcctacgcaaagcttagtagcatctttgaaatcgtttggctggtcgatccgccatttcccctggtagacctggcagatgaggctagaaataccccactggcgacagtgttctagcctgcgtggctgcctgcacaccctatgggtgtgaagccaaacaatggacaaggtgtgaagagccccgtgtgctcgctttgagtcctccggcccctgaatgtggctaaccttaaccctgcagctagagcacgtaacccaatgtgtatctagtcgtaatgagcaattgcgggatgggaccaactactttgggtgtccgtgtttcactttttcctttatatttgcttatggtgacaatatatacaatatatatattggcaccatgggagctcaggtgtctacacaaaagacgggtgcacatgagactggcttgaatgccagcggcaattctgtcatccattacaccaacatcaattactacaaggatgctgcatccaactccgcaaataggcaagacttcacacaagacccaggtaagttcactgaacctgtaaaggacatcatggtgaaaactatgcctgcgttgaactcaccctctgctgaggaatgcggctacagtgacagagtgcgttccatcacattaggtaattccactatcaccacccaagagtgtgccaacgtggtcgtcgggtacggagtgtggccagagtatctaaaagacaacgaggccacagccgaagatcagcccacacaacctgatgtggccacctgccgcttctatactttagaatcagtgcagtggatgaaaaattcggccgggtggtggtggaagctgcctgatgcactgtcacagatgggcttgtttggtcaaaacatgcagtaccactacttgggaagaacagggtatactatacacgtacaatgcaatgcctcaaagttccatcaaggatgcctgcttgtagtgtgtgtgcctgaagctgagatggggtgctctaacttgaacaacacgcccgaattcagtgagttatccggaggagacagtgccagaatgttcaccgatacacaggtcggggagtcaaacgccaagaaagtacagacagcggtgtggaacgccggaatgggcgttggagtgggtaatctcactattttccctcaccaatggatcaacctgaggaccaacaacagtgcaacgctagtgatgccttacataaacagcgtccccatggataacatgttcaggcacaataacttaacactaatgattatcccatttgtaccgcttaattacagcgagggatcctcaccgtacgtgcccataacagtcaccatcgctcctatgtgtgcagagtacaacgggctacggctggccagcaaccagggtctaccagttatgacaacacctgggagcacgcaatttctaacttcagatgacttccagtcaccgtcagcgatgccacagtttgatgtcaccccagagatgcaaataccaggccgcgtaaataatttgatggagattgccgaggtggactcagtggtgcctgtgaataacacagaggacaacgtgagtagccttaaggcataccagattccagtacagtccaacagtgacaatggtaagcaggtttttggttttcccttacaaccgggtgccaacaacgttctgaatagaaccctcttgggtgaaattctaaactattacacccattggagtggcagcataaaacttacattcatgttctgtgggtctgccatggccactggcaagttccttctggcatactcaccacctggagcaggagtcccgaaaaaccgaaaagatgctatgctgggcacccacgtcatatgggatgttgggttacaatcaagctgcgtgttgtgcgtaccgtggattagccaaacacactacagatacgtggttgaggatgaatacacagcagccggatatgttacatgctggtatcaaacaaatatcgtggtgccagctgatgtgcaaagctcatgtgacatcttgtgctttgtttcagcctgcaacgatttctctgtgcggatgctgaaagatacgccctttataagacaggacacattttatcacggcccagtggaggagtctgtggatcgtgcagtggcacgtgtggcggacacgattagttcacggcccactaactcggagtcaattccagcgctcaccgccgccgagactgggcatacctcccaagttgtgcctagcgacaccatgcaaacaagacatgtgaaaaactaccactcacggtccgaatcgtccattgaaaatttcctatgccggtctgcctgtgtatactatgccacatacaccaataactcaaaaaaagaggggtttgcagagtgggttatcaacactagacaggtggcacagctaagaagaaagttggagctcttcacatatctaaggtttgacttagaactaacatttgttataacgagcgcacaacaacccagtaccgcctccagtgtagacgctcccgtgcaaacccatcagattatgtatgtgcctccaggtggccctgtaccaacaaaggttaaagattatgcatggcaaacttccacaaaccccagcgttttctggacagaaggaaacgccccaccaaggatgtccatcccattcattagcattggtaatgcgtatagttgtttttatgatgggtggacgcagttttcaagaaacggggtctatggcatcaataccctcaacaacatgggcacgttgtatatgaggcatgtcaatgaagcggggcagggtccaatcaaaagtactgttagaatatatttcaaacccaagcacgtgaaggcgtgggtgccacgtccgccaagattgtgtcaatatgagaagcagaaaaatgtcaattttagccctataggagtaaccacaagtagaacggacattataacaacaggcacttttggtcagcagtccggagcgatatatgtgggcaattacaggatagtcaataggcacctggcaacacactttgactggcaaaattgcatatgggaggattacaacagggacctcctagtatgcactacaacggcgcacggttgtgacaatattgcaagatgccagtgcacagcaggtgtgtattattgtgcttctaggaacaagcattacccggtgcattttgaaggaccaggcttggtggaggtccaggaaagtgaatattatccaaaaaggtatcaatcacacgtgcttcttgccgcagggttttctgagccgggggattgtggtggtattctcaggtgtgaacatggtgttataggcatcgtgaccatgggtggtgagggtgttgtcggctttgccgatgtgcgcgatctcttatggctggaagacgacgcaatggaacagggagtcagggactacgtggaacagcttggaaacgcctttggttctgggtttaccaaccagatttgcgagcaagtcaatctgttaaaagagtcactaataggccaagactccatcttggaaaaatcactcaaggcattagtgaaaatcatatcagcactggttatcgtggtgagaaaccacgatgacctcataacagtcactgccaccttagccctgattggctgtaccacttcaccgtggcggtggctaaagaaaaaagtgactcaatactatgggattcccatggccgagagacagaacaatggctggctcaagaaattcacagaaatgaccaatgcatgcaagggcatggaatggatcgccatcaaaatccaaaagttcattgagtggctcaaaattaagatcttgccagaagtaaaggaaaaacacgagtttcttaccagacttaagcaactcccactcttagaaagccaaattgccaccatcgagcagagtgcaccatcacagagtgatcaggaacaactgttctccaatatacagtacttcgcgcactattgcagaaagtacgccccactgtacgcagctgaggcaaaaagagtgttctctctagaaaagaaaatgagcaactacatacagttcaagtccaaatgccgtattgaaccagtctgtttattgctccatggtagccctggagccggaaaatccgtggctaccaatttgattggacgctcacttgcagagaagctcaacagttcagtgtattcattgccaccagacccagaccattttgacggctataaacaacaagctgtcgtaatcatggatgacctgtgccagaacccagatgggaaagacgtgtccttgttctgccagatggtttcaagtgtagactttgttccaccaatggcagcacttgaggagaaaggcatactcttcacatcgcctttcgtgctcgcctctaccaacgcaggttccatcaacgctcccactgtgtcagacagcagagcccttgctagaaggttccacttcgacttgaacattgaggtcatctcaatgtacagccagaatggcaaaatcaacatgccaatgtcagtgaaggcttgtgatgatgagtgttgtccagttaatttcaagaggtgttgtccattagtgtgcggtaaggccattcagttcattgacagaagaacacaaatgaggtattcactagacatgcttgtaactgaaatgttcagggagtacaaccacagacatagtgtgggggcaacactcgaggcactcttccaaggcccacccgtgtacaaagagatcaagatcagtgttgcgccagaaacccctcccccaccggcaatagcagatctgctgaaatctgtggacagtgaagcagttagagagtactgcaaggaaaagggatggttggtgcctgagatcaattccactctacaaattgaaaagcacgtgagcagggccttcatttgcctgcaagcgctcaccacgtttgtttccgtggcggggataatttacatcatatacaaattgtttgctggcttccagggggcctacaccggtatgccaaaccaaaaacctaaagtaccaactttgaggcaggctaaggtgcagggaccggcattcgaattcgctgtggcaatgatgaaaagaaatgctagcacagtgaagaccgagtacggtgagttcacaatgcttggaatctatgacaggtgggcagtgctacctcgtcacgctagaccagggcctaccatcctaatgaatgaccaggaagtgggtgtggtggatgccaaggagttagtggacaaggatggcaccaatttagaattaacactcttgaagctcaatagaaatgagaaattcagagacatccgaggcttcttaacacgtgaggaggccgaagtcaacgaggctgtgcttgctattaacaccagcaagttcccaaatatgtacattccagttgggcaagtaacagattatggcttcctcaacttgggtggcacaccaactaagcgcatgctgatgtacaacttcccaacacgtgcaggccaatgtgggggagttctaatgtctacaggtaaagtgctcggaatacacgtcggtggtaatggacaccaagggttttcggcagccctgttacgccactatttcaatgacgagcagggagagattgagttcatcgagagctctaaggatgcaggctttccagtcattaacacacctagcaaaacaaagctcgagcccagtgttttccatcacgtgtttgagggaaacaaagaaccagcagtgttgaggaatggagacccaagactgaaagccaattttgaggaagccattttctccaagtacatcgggaatgtgaacacacatgtagacgagtacatgatggaagccgtcgaccattatgcaggacagttagctactttagatatcagtacagaacctatgaagcttgaggatgctgtctacggtactgagggcttggaagctctggacttgaccactagtgctgggtatccctatgtcgcccttggtataaaaaagagagacatcttatccaaaaagtctaaggacctgtctaaactgagagagtgtatggacaagtatgggttaaacctccctatggtcacctatgtgaaggatgaattgaggtcagcagagaaagtggctaagggcaaatccagactaatcgaggcgtccagtttaaatgactcagtggcaatgagacagactttcggtaacctgtataagacattccatctgaacccgggcatagtgactggcagtgcagttggatgcgatcccgacttgttctggagcaagattccagtcatgctcgatggtcacctcatcgcctttgactacagtggatatgatgccagcctgagtccggtgtggtttgcttgcttgaaactgttgttggaaaagctggggtactcacacaaggaaactaattacatagattacttgtgcaactcccaccatttgtacagagacaagcactactttgtgaggggaggaatgccatccgggtgttctggaaccagcattttcaattcaatgattaataacataatcatcagaacactgatgttgaaggtgtacaaagggatagatctagaccaatttaggatgattgcatatggtgacgacgtcattgcttcgtacccgtatccaattgatgcgtcattgctggctgaggctggcaaggggtatggtctgattatgacaccagcagacaaaggagagtgtttcaatgaagtaacgtggaccaacgtcactttcctgaaaagatacttcagagcggatgagcaatatcccttcctcgttcacccagtgatgccaatgaaggacattcatgagtcaattaggtggactaaagaccccaaaaacacccaagatcacgtgcgatccttgtgcttattggcctggcacaatggagagcaagaatatgagaattttgtatccaaaatcagaagcgtcccagttgggcgctgcttgacattgcctgcgttctcgactctgcgcaggaagtggttggattccttctaaaattgaagtacaatgtgatttaactcaattggcttaaccctaccacacttaccgaactagataacggtgtggtaggggtaaattc

SEQ ID NO: 9 - DNA, 7473 bp, Artificial Sequence
  cDNA sequence of CVB1-miR133&206T
tcctgtgggttgatcccacccacagggcccactgggcgctagcacactggtattccggtacctttgtgcgcctgttttatacacccttcccaagtgtaacttagaagcttatcacacacggtcaacaggtgactcagtatgccaactgggtcttgatcaagcacttctgtttccccggactgagtatcaataagctgctcacgcggctgaaggagaaaacgttcgttaaccggccaattacttcgagaaacctagtaacaccatggaggttgcgcagtgtttcgctccacacaaccccagtgtagatcaggccgatgagtcaccgcactcctcacgggcgaccgtggcggtggctgcgctggcggcctgcccatgggataacccatgggacgcttcaatactgacatggtgcgaagagtctattgagctaattggtagtcctccggcccctgaatgcggctaatcctaactgcggagcagatacccacacgccagtgggcagtctgtcgtaatgggcaactctgcagcggaaccgactactttgggtgtccgtgtttccttttatttttatactggctgcttatggtgacaattgagagattgttaccatatagctattggattggccatccagtgacaaacagagcgattatttatttatttgttggttatatcccattaagccaaaaggccctagttactctcaactttatactattgcttaatacaacgaaatgggagctcaggtgtctacacaaaagacgggtgcacatgagactggcttgaatgccagcggcaattctgtcatccattacaccaacatcaattactacaaggatgctgcatccaactccgcaaataggcaagacttcacacaagacccaggtaagttcactgaacctgtaaaggacatcatggtgaaaactatgcctgcgttgaactcaccctctgctgaggaatgcggctacagtgacagagtgcgttccatcacattaggtaattccactatcaccacccaagagtgtgccaacgtggtcgtcgggtacggagtgtggccagagtatctaaaagacaacgaggccacagccgaagatcagcccacacaacctgatgtggccacctgccgcttctatactttagaatcagtgcagtggatgaaaaattcggccgggtggtggtggaagctgcctgatgcactgtcacagatgggcttgtttggtcaaaacatgcagtaccactacttgggaagaacagggtatactatacacgtacaatgcaatgcctcaaagttccatcaaggatgcctgcttgtagtgtgtgtgcctgaagctgagatggggtgctctaacttgaacaacacgcccgaattcagtgagttatccggaggagacagtgccagaatgttcaccgatacacaggtcggggagtcaaacgccaagaaagtacagacagcggtgtggaacgccggaatgggcgttggagtgggtaatctcactattttccctcaccaatggatcaacctgaggaccaacaacagtgcaacgctagtgatgccttacataaacagcgtccccatggataacatgttcaggcacaataacttaacactaatgattatcccatttgtaccgcttaattacagcgagggatcctcaccgtacgtgcccataacagtcaccatcgctcctatgtgtgcagagtacaacgggctacggctggccagcaaccagggtctaccagttatgacaacacctgggagcacgcaatttctaacttcagatgacttccagtcaccgtcagcgatgccacagtttgatgtcaccccagagatgcaaataccaggccgcgtaaataatttgatggagattgccgaggtggactcagtggtgcctgtgaataacacagaggacaacgtgagtagccttaaggcataccagattccagtacagtccaacagtgacaatggtaagcaggtttttggttttcccttacaaccgggtgccaacaacgttctgaatagaaccctcttgggtgaaattctaaactattacacccattggagtggcagcataaaacttacattcatgttctgtgggtctgccatggccactggcaagttccttctggcatactcaccacctggagcaggagtcccgaaaaaccgaaaagatgctatgctgggcacccacgtcatatgggatgttgggttacaatcaagctgcgtgttgtgcgtaccgtggattagccaaacacactacagatacgtggttgaggatgaatacacagcagccggatatgttacatgctggtatcaaacaaatatcgtggtgccagctgatgtgcaaagctcatgtgacatcttgtgctttgtttcagcctgcaacgatttctctgtgcggatgctgaaagatacgccctttataagacaggacacattttatcacggcccagtggaggagtctgtggatcgtgcagtggcacgtgtggcggacacgattagttcacggcccactaactcggagtcaattccagcgctcaccgccgccgagactgggcatacctcccaagttgtgcctagcgacaccatgcaaacaagacatgtgaaaaactaccactcacggtccgaatcgtccattgaaaatttcctatgccggtctgcctgtgtatactatgccacatacaccaataactcaaaaaaagaggggtttgcagagtgggttatcaacactagacaggtggcacagctaagaagaaagttggagctcttcacatatctaaggtttgacttagaactaacatttgttataacgagcgcacaacaacccagtaccgcctccagtgtagacgctcccgtgcaaacccatcagattatgtatgtgcctccaggtggccctgtaccaacaaaggttaaagattatgcatggcaaacttccacaaaccccagcgttttctggacagaaggaaacgccccaccaaggatgtccatcccattcattagcattggtaatgcgtatagttgtttttatgatgggtggacgcagttttcaagaaacggggtctatggcatcaataccctcaacaacatgggcacgttgtatatgaggcatgtcaatgaagcggggcagggtccaatcaaaagtactgttagaatatatttcaaacccaagcacgtgaaggcgtgggtgccacgtccgccaagattgtgtcaatatgagaagcagaaaaatgtcaattttagccctataggagtaaccacaagtagaacggacattataacaacaggcacttttggtcagcagtccggagcgatatatgtgggcaattacaggatagtcaataggcacctggcaacacactttgactggcaaaattgcatatgggaggattacaacagggacctcctagtatgcactacaacggcgcacggttgtgacaatattgcaagatgccagtgcacagcaggtgtgtattattgtgcttctaggaacaagcattacccggtgcattttgaaggaccaggcttggtggaggtccaggaaagtgaatattatccaaaaaggtatcaatcacacgtgcttcttgccgcagggttttctgagccgggggattgtggtggtattctcaggtgtgaacatggtgttataggcatcgtgaccatgggtggtgagggtgttgtcggctttgccgatgtgcgcgatctcttatggctggaagacgacgcaatggaacagggagtcagggactacgtggaacagcttggaaacgcctttggttctgggtttaccaaccagatttgcgagcaagtcaatctgttaaaagagtcactaataggccaagactccatcttggaaaaatcactcaaggcattagtgaaaatcatatcagcactggttatcgtggtgagaaaccacgatgacctcataacagtcactgccaccttagccctgattggctgtaccacttcaccgtggcggtggctaaagaaaaaagtgactcaatactatgggattcccatggccgagagacagaacaatggctggctcaagaaattcacagaaatgaccaatgcatgcaagggcatggaatggatcgccatcaaaatccaaaagttcattgagtggctcaaaattaagatcttgccagaagtaaaggaaaaacacgagtttcttaccagacttaagcaactcccactcttagaaagccaaattgccaccatcgagcagagtgcaccatcacagagtgatcaggaacaactgttctccaatatacagtacttcgcgcactattgcagaaagtacgccccactgtacgcagctgaggcaaaaagagtgttctctctagaaaagaaaatgagcaactacatacagttcaagtccaaatgccgtattgaaccagtctgtttattgctccatggtagccctggagccggaaaatccgtggctaccaatttgattggacgctcacttgcagagaagctcaacagttcagtgtattcattgccaccagacccagaccattttgacggctataaacaacaagctgtcgtaatcatggatgacctgtgccagaacccagatgggaaagacgtgtccttgttctgccagatggtttcaagtgtagactttgttccaccaatggcagcacttgaggagaaaggcatactcttcacatcgcctttcgtgctcgcctctaccaacgcaggttccatcaacgctcccactgtgtcagacagcagagcccttgctagaaggttccacttcgacttgaacattgaggtcatctcaatgtacagccagaatggcaaaatcaacatgccaatgtcagtgaaggcttgtgatgatgagtgttgtccagttaatttcaagaggtgttgtccattagtgtgcggtaaggccattcagttcattgacagaagaacacaaatgaggtattcactagacatgcttgtaactgaaatgttcagggagtacaaccacagacatagtgtgggggcaacactcgaggcactcttccaaggcccacccgtgtacaaagagatcaagatcagtgttgcgccagaaacccctcccccaccggcaatagcagatctgctgaaatctgtggacagtgaagcagttagagagtactgcaaggaaaagggatggttggtgcctgagatcaattccactctacaaattgaaaagcacgtgagcagggccttcatttgcctgcaagcgctcaccacgtttgtttccgtggcggggataatttacatcatatacaaattgtttgctggcttccagggggcctacaccggtatgccaaaccaaaaacctaaagtaccaactttgaggcaggctaaggtgcagggaccggcattcgaattcgctgtggcaatgatgaaaagaaatgctagcacagtgaagaccgagtacggtgagttcacaatgcttggaatctatgacaggtgggcagtgctacctcgtcacgctagaccagggcctaccatcctaatgaatgaccaggaagtgggtgtggtggatgccaaggagttagtggacaaggatggcaccaatttagaattaacactcttgaagctcaatagaaatgagaaattcagagacatccgaggcttcttaacacgtgaggaggccgaagtcaacgaggctgtgcttgctattaacaccagcaagttcccaaatatgtacattccagttgggcaagtaacagattatggcttcctcaacttgggtggcacaccaactaagcgcatgctgatgtacaacttcccaacacgtgcaggccaatgtgggggagttctaatgtctacaggtaaagtgctcggaatacacgtcggtggtaatggacaccaagggttttcggcagccctgttacgccactatttcaatgacgagcagggagagattgagttcatcgagagctctaaggatgcaggctttccagtcattaacacacctagcaaaacaaagctcgagcccagtgttttccatcacgtgtttgagggaaacaaagaaccagcagtgttgaggaatggagacccaagactgaaagccaattttgaggaagccattttctccaagtacatcgggaatgtgaacacacatgtagacgagtacatgatggaagccgtcgaccattatgcaggacagttagctactttagatatcagtacagaacctatgaagcttgaggatgctgtctacggtactgagggcttggaagctctggacttgaccactagtgctgggtatccctatgtcgcccttggtataaaaaagagagacatcttatccaaaaagtctaaggacctgtctaaactgagagagtgtatggacaagtatgggttaaacctccctatggtcacctatgtgaaggatgaattgaggtcagcagagaaagtggctaagggcaaatccagactaatcgaggcgtccagtttaaatgactcagtggcaatgagacagactttcggtaacctgtataagacattccatctgaacccgggcatagtgactggcagtgcagttggatgcgatcccgacttgttctggagcaagattccagtcatgctcgatggtcacctcatcgcctttgactacagtggatatgatgccagcctgagtccggtgtggtttgcttgcttgaaactgttgttggaaaagctggggtactcacacaaggaaactaattacatagattacttgtgcaactcccaccatttgtacagagacaagcactactttgtgaggggaggaatgccatccgggtgttctggaaccagcattttcaattcaatgattaataacataatcatcagaacactgatgttgaaggtgtacaaagggatagatctagaccaatttaggatgattgcatatggtgacgacgtcattgcttcgtacccgtatccaattgatgcgtcattgctggctgaggctggcaaggggtatggtctgattatgacaccagcagacaaaggagagtgtttcaatgaagtaacgtggaccaacgtcactttcctgaaaagatacttcagagcggatgagcaatatcccttcctcgttcacccagtgatgccaatgaaggacattcatgagtcaattaggtggactaaagaccccaaaaacacccaagatcacgtgcgatccttgtgcttattggcctggcacaatggagagcaagaatatgagaattttgtatccaaaatcagaagcgtcccagttgggcgctgcttgacattgcctgcgttctcgactctgcgcaggaagtggttggattccttctaaaatacagctggttgaaggggaccaacgatacagctggttgaaggggaccaaaccggtccacacacttccttacattccatcacccacacacttccttacattccatgaagtacaatgtgatttaactcaattggcttaaccctaccacacttaccgaactagataacggtgtggtaggggtaaattc

SEQ ID NO: 10 - DNA, 7848 bp, Artificial Sequence
  cDNA sequence of CVB1-GM-CSF
tcctgtgggttgatcccacccacagggcccactgggcgctagcacactggtattccggtacctttgtgcgcctgttttatacacccttcccaagtgtaacttagaagcttatcacacacggtcaacaggtgactcagtatgccaactgggtcttgatcaagcacttctgtttccccggactgagtatcaataagctgctcacgcggctgaaggagaaaacgttcgttaaccggccaattacttcgagaaacctagtaacaccatggaggttgcgcagtgtttcgctccacacaaccccagtgtagatcaggccgatgagtcaccgcactcctcacgggcgaccgtggcggtggctgcgctggcggcctgcccatgggataacccatgggacgcttcaatactgacatggtgcgaagagtctattgagctaattggtagtcctccggcccctgaatgcggctaatcctaactgcggagcagatacccacacgccagtgggcagtctgtcgtaatgggcaactctgcagcggaaccgactactttgggtgtccgtgtttccttttatttttatactggctgcttatggtgacaattgagagattgttaccatatagctattggattggccatccagtgacaaacagagcgattatttatttatttgttggttatatcccattaagccaaaaggccctagttactctcaactttatactattgcttaatacaacgaaatgggagctcaggtgtctacacaaaagacgggtgcacatgagactggcttgaatgccagcggcaattctgtcatccattacaccaacatcaattactacaaggatgctgcatccaactccgcaaataggcaagacttcacacaagacccaggtaagttcactgaacctgtaaaggacatcatggtgaaaactatgcctgcgttgaactcaccctctgctgaggaatgcggctacagtgacagagtgcgttccatcacattaggtaattccactatcaccacccaagagtgtgccaacgtggtcgtcgggtacggagtgtggccagagtatctaaaagacaacgaggccacagccgaagatcagcccacacaacctgatgtggccacctgccgcttctatactttagaatcagtgcagtggatgaaaaattcggccgggtggtggtggaagctgcctgatgcactgtcacagatgggcttgtttggtcaaaacatgcagtaccactacttgggaagaacagggtatactatacacgtacaatgcaatgcctcaaagttccatcaaggatgcctgcttgtagtgtgtgtgcctgaagctgagatggggtgctctaacttgaacaacacgcccgaattcagtgagttatccggaggagacagtgccagaatgttcaccgatacacaggtcggggagtcaaacgccaagaaagtacagacagcggtgtggaacgccggaatgggcgttggagtgggtaatctcactattttccctcaccaatggatcaacctgaggaccaacaacagtgcaacgctagtgatgccttacataaacagcgtccccatggataacatgttcaggcacaataacttaacactaatgattatcccatttgtaccgcttaattacagcgagggatcctcaccgtacgtgcccataacagtcaccatcgctcctatgtgtgcagagtacaacgggctacggctggccagcaaccagggtctaccagttatgacaacacctgggagcacgcaatttctaacttcagatgacttccagtcaccgtcagcgatgccacagtttgatgtcaccccagagatgcaaataccaggccgcgtaaataatttgatggagattgccgaggtggactcagtggtgcctgtgaataacacagaggacaacgtgagtagccttaaggcataccagattccagtacagtccaacagtgacaatggtaagcaggtttttggttttcccttacaaccgggtgccaacaacgttctgaatagaaccctcttgggtgaaattctaaactattacacccattggagtggcagcataaaacttacattcatgttctgtgggtctgccatggccactggcaagttccttctggcatactcaccacctggagcaggagtcccgaaaaaccgaaaagatgctatgctgggcacccacgtcatatgggatgttgggttacaatcaagctgcgtgttgtgcgtaccgtggattagccaaacacactacagatacgtggttgaggatgaatacacagcagccggatatgttacatgctggtatcaaacaaatatcgtggtgccagctgatgtgcaaagctcatgtgacatcttgtgctttgtttcagcctgcaacgatttctctgtgcggatgctgaaagatacgccctttataagacaggacacattttatcacggcccagtggaggagtctgtggatcgtgcagtggcacgtgtggcggacacgattagttcacggcccactaactcggagtcaattccagcgctcaccgccgccgagactgggcatacctcccaagttgtgcctagcgacaccatgcaaacaagacatgtgaaaaactaccactcacggtccgaatcgtccattgaaaatttcctatgccggtctgcctgtgtatactatgccacatacaccaataactcaaaaaaagaggggtttgcagagtgggttatcaacactagacaggtggcacagctaagaagaaagttggagctcttcacatatctaaggtttgacttagaactaacatttgttataacgagcgcacaacaacccagtaccgcctccagtgtagacgctcccgtgcaaacccatcagattatgtatgtgcctccaggtggccctgtaccaacaaaggttaaagattatgcatggcaaacttccacaaaccccagcgttttctggacagaaggaaacgccccaccaaggatgtccatcccattcattagcattggtaatgcgtatagttgtttttatgatgggtggacgcagttttcaagaaacggggtctatggcatcaataccctcaacaacatgggcacgttgtatatgaggcatgtcaatgaagcggggcagggtccaatcaaaagtactgttagaatatatttcaaacccaagcacgtgaaggcgtgggtgccacgtccgccaagattgtgtcaatatgagaagcagaaaaatgtcaattttagccctataggagtaaccacaagtagaacggacattataacaacaggcacttttggtcagcagtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagaccacaagtagaacggacattataacaacaggcacttttggtcagcagtccggagcgatatatgtgggcaattacaggatagtcaataggcacctggcaacacactttgactggcaaaattgcatatgggaggattacaacagggacctcctagtatgcactacaacggcgcacggttgtgacaatattgcaagatgccagtgcacagcaggtgtgtattattgtgcttctaggaacaagcattacccggtgcattttgaaggaccaggcttggtggaggtccaggaaagtgaatattatccaaaaaggtatcaatcacacgtgcttcttgccgcagggttttctgagccgggggattgtggtggtattctcaggtgtgaacatggtgttataggcatcgtgaccatgggtggtgagggtgttgtcggctttgccgatgtgcgcgatctcttatggctggaagacgacgcaatggaacagggagtcagggactacgtggaacagcttggaaacgcctttggttctgggtttaccaaccagatttgcgagcaagtcaatctgttaaaagagtcactaataggccaagactccatcttggaaaaatcactcaaggcattagtgaaaatcatatcagcactggttatcgtggtgagaaaccacgatgacctcataacagtcactgccaccttagccctgattggctgtaccacttcaccgtggcggtggctaaagaaaaaagtgactcaatactatgggattcccatggccgagagacagaacaatggctggctcaagaaattcacagaaatgaccaatgcatgcaagggcatggaatggatcgccatcaaaatccaaaagttcattgagtggctcaaaattaagatcttgccagaagtaaaggaaaaacacgagtttcttaccagacttaagcaactcccactcttagaaagccaaattgccaccatcgagcagagtgcaccatcacagagtgatcaggaacaactgttctccaatatacagtacttcgcgcactattgcagaaagtacgccccactgtacgcagctgaggcaaaaagagtgttctctctagaaaagaaaatgagcaactacatacagttcaagtccaaatgccgtattgaaccagtctgtttattgctccatggtagccctggagccggaaaatccgtggctaccaatttgattggacgctcacttgcagagaagctcaacagttcagtgtattcattgccaccagacccagaccattttgacggctataaacaacaagctgtcgtaatcatggatgacctgtgccagaacccagatgggaaagacgtgtccttgttctgccagatggtttcaagtgtagactttgttccaccaatggcagcacttgaggagaaaggcatactcttcacatcgcctttcgtgctcgcctctaccaacgcaggttccatcaacgctcccactgtgtcagacagcagagcccttgctagaaggttccacttcgacttgaacattgaggtcatctcaatgtacagccagaatggcaaaatcaacatgccaatgtcagtgaaggcttgtgatgatgagtgttgtccagttaatttcaagaggtgttgtccattagtgtgcggtaaggccattcagttcattgacagaagaacacaaatgaggtattcactagacatgcttgtaactgaaatgttcagggagtacaaccacagacatagtgtgggggcaacactcgaggcactcttccaaggcccacccgtgtacaaagagatcaagatcagtgttgcgccagaaacccctcccccaccggcaatagcagatctgctgaaatctgtggacagtgaagcagttagagagtactgcaaggaaaagggatggttggtgcctgagatcaattccactctacaaattgaaaagcacgtgagcagggccttcatttgcctgcaagcgctcaccacgtttgtttccgtggcggggataatttacatcatatacaaattgtttgctggcttccagggggcctacaccggtatgccaaaccaaaaacctaaagtaccaactttgaggcaggctaaggtgcagggaccggcattcgaattcgctgtggcaatgatgaaaagaaatgctagcacagtgaagaccgagtacggtgagttcacaatgcttggaatctatgacaggtgggcagtgctacctcgtcacgctagaccagggcctaccatcctaatgaatgaccaggaagtgggtgtggtggatgccaaggagttagtggacaaggatggcaccaatttagaattaacactcttgaagctcaatagaaatgagaaattcagagacatccgaggcttcttaacacgtgaggaggccgaagtcaacgaggctgtgcttgctattaacaccagcaagttcccaaatatgtacattccagttgggcaagtaacagattatggcttcctcaacttgggtggcacaccaactaagcgcatgctgatgtacaacttcccaacacgtgcaggccaatgtgggggagttctaatgtctacaggtaaagtgctcggaatacacgtcggtggtaatggacaccaagggttttcggcagccctgttacgccactatttcaatgacgagcagggagagattgagttcatcgagagctctaaggatgcaggctttccagtcattaacacacctagcaaaacaaagctcgagcccagtgttttccatcacgtgtttgagggaaacaaagaaccagcagtgttgaggaatggagacccaagactgaaagccaattttgaggaagccattttctccaagtacatcgggaatgtgaacacacatgtagacgagtacatgatggaagccgtcgaccattatgcaggacagttagctactttagatatcagtacagaacctatgaagcttgaggatgctgtctacggtactgagggcttggaagctctggacttgaccactagtgctgggtatccctatgtcgcccttggtataaaaaagagagacatcttatccaaaaagtctaaggacctgtctaaactgagagagtgtatggacaagtatgggttaaacctccctatggtcacctatgtgaaggatgaattgaggtcagcagagaaagtggctaagggcaaatccagactaatcgaggcgtccagtttaaatgactcagtggcaatgagacagactttcggtaacctgtataagacattccatctgaacccgggcatagtgactggcagtgcagttggatgcgatcccgacttgttctggagcaagattccagtcatgctcgatggtcacctcatcgcctttgactacagtggatatgatgccagcctgagtccggtgtggtttgcttgcttgaaactgttgttggaaaagctggggtactcacacaaggaaactaattacatagattacttgtgcaactcccaccatttgtacagagacaagcactactttgtgaggggaggaatgccatccgggtgttctggaaccagcattttcaattcaatgattaataacataatcatcagaacactgatgttgaaggtgtacaaagggatagatctagaccaatttaggatgattgcatatggtgacgacgtcattgcttcgtacccgtatccaattgatgcgtcattgctggctgaggctggcaaggggtatggtctgattatgacaccagcagacaaaggagagtgtttcaatgaagtaacgtggaccaacgtcactttcctgaaaagatacttcagagcggatgagcaatatcccttcctcgttcacccagtgatgccaatgaaggacattcatgagtcaattaggtggactaaagaccccaaaaacacccaagatcacgtgcgatccttgtgcttattggcctggcacaatggagagcaagaatatgagaattttgtatccaaaatcagaagcgtcccagttgggcgctgcttgacattgcctgcgttctcgactctgcgcaggaagtggttggattccttctaaaattgaagtacaatgtgatttaactcaattggcttaaccctaccacacttaccgaactagataacggtgtggtaggggtaaattc

SEQ ID NO: 11 - DNA, 8214 bp, Artificial Sequence
  cDNA sequence of CVB1-Anti-PD1
tcctgtgggttgatcccacccacagggcccactgggcgctagcacactggtattccggtacctttgtgcgcctgttttatacacccttcccaagtgtaacttagaagcttatcacacacggtcaacaggtgactcagtatgccaactgggtcttgatcaagcacttctgtttccccggactgagtatcaataagctgctcacgcggctgaaggagaaaacgttcgttaaccggccaattacttcgagaaacctagtaacaccatggaggttgcgcagtgtttcgctccacacaaccccagtgtagatcaggccgatgagtcaccgcactcctcacgggcgaccgtggcggtggctgcgctggcggcctgcccatgggataacccatgggacgcttcaatactgacatggtgcgaagagtctattgagctaattggtagtcctccggcccctgaatgcggctaatcctaactgcggagcagatacccacacgccagtgggcagtctgtcgtaatgggcaactctgcagcggaaccgactactttgggtgtccgtgtttccttttatttttatactggctgcttatggtgacaattgagagattgttaccatatagctattggattggccatccagtgacaaacagagcgattatttatttatttgttggttatatcccattaagccaaaaggccctagttactctcaactttatactattgcttaatacaacgaaatgggagctcaggtgtctacacaaaagacgggtgcacatgagactggcttgaatgccagcggcaattctgtcatccattacaccaacatcaattactacaaggatgctgcatccaactccgcaaataggcaagacttcacacaagacccaggtaagttcactgaacctgtaaaggacatcatggtgaaaactatgcctgcgttgaactcaccctctgctgaggaatgcggctacagtgacagagtgcgttccatcacattaggtaattccactatcaccacccaagagtgtgccaacgtggtcgtcgggtacggagtgtggccagagtatctaaaagacaacgaggccacagccgaagatcagcccacacaacctgatgtggccacctgccgcttctatactttagaatcagtgcagtggatgaaaaattcggccgggtggtggtggaagctgcctgatgcactgtcacagatgggcttgtttggtcaaaacatgcagtaccactacttgggaagaacagggtatactatacacgtacaatgcaatgcctcaaagttccatcaaggatgcctgcttgtagtgtgtgtgcctgaagctgagatggggtgctctaacttgaacaacacgcccgaattcagtgagttatccggaggagacagtgccagaatgttcaccgatacacaggtcggggagtcaaacgccaagaaagtacagacagcggtgtggaacgccggaatgggcgttggagtgggtaatctcactattttccctcaccaatggatcaacctgaggaccaacaacagtgcaacgctagtgatgccttacataaacagcgtccccatggataacatgttcaggcacaataacttaacactaatgattatcccatttgtaccgcttaattacagcgagggatcctcaccgtacgtgcccataacagtcaccatcgctcctatgtgtgcagagtacaacgggctacggctggccagcaaccagggtctaccagttatgacaacacctgggagcacgcaatttctaacttcagatgacttccagtcaccgtcagcgatgccacagtttgatgtcaccccagagatgcaaataccaggccgcgtaaataatttgatggagattgccgaggtggactcagtggtgcctgtgaataacacagaggacaacgtgagtagccttaaggcataccagattccagtacagtccaacagtgacaatggtaagcaggtttttggttttcccttacaaccgggtgccaacaacgttctgaatagaaccctcttgggtgaaattctaaactattacacccattggagtggcagcataaaacttacattcatgttctgtgggtctgccatggccactggcaagttccttctggcatactcaccacctggagcaggagtcccgaaaaaccgaaaagatgctatgctgggcacccacgtcatatgggatgttgggttacaatcaagctgcgtgttgtgcgtaccgtggattagccaaacacactacagatacgtggttgaggatgaatacacagcagccggatatgttacatgctggtatcaaacaaatatcgtggtgccagctgatgtgcaaagctcatgtgacatcttgtgctttgtttcagcctgcaacgatttctctgtgcggatgctgaaagatacgccctttataagacaggacacattttatcacggcccagtggaggagtctgtggatcgtgcagtggcacgtgtggcggacacgattagttcacggcccactaactcggagtcaattccagcgctcaccgccgccgagactgggcatacctcccaagttgtgcctagcgacaccatgcaaacaagacatgtgaaaaactaccactcacggtccgaatcgtccattgaaaatttcctatgccggtctgcctgtgtatactatgccacatacaccaataactcaaaaaaagaggggtttgcagagtgggttatcaacactagacaggtggcacagctaagaagaaagttggagctcttcacatatctaaggtttgacttagaactaacatttgttataacgagcgcacaacaacccagtaccgcctccagtgtagacgctcccgtgcaaacccatcagattatgtatgtgcctccaggtggccctgtaccaacaaaggttaaagattatgcatggcaaacttccacaaaccccagcgttttctggacagaaggaaacgccccaccaaggatgtccatcccattcattagcattggtaatgcgtatagttgtttttatgatgggtggacgcagttttcaagaaacggggtctatggcatcaataccctcaacaacatgggcacgttgtatatgaggcatgtcaatgaagcggggcagggtccaatcaaaagtactgttagaatatatttcaaacccaagcacgtgaaggcgtgggtgccacgtccgccaagattgtgtcaatatgagaagcagaaaaatgtcaattttagccctataggagtaaccacaagtagaacggacattataacaacaggcacttttggtcagcagatgaagcacctgtggttcttcctgctgctggtggccgctcctaggtgggtgctgtcccaggtgcagctggtgcagagcggcgtggaggtgaagaagcccggcgcttccgtgaaggtgtcctgcaaggcctccggctacaccttcaccaactactacatgtactgggtgaggcaggcccctggacagggactggagtggatgggcggcatcaacccttccaacggcggcaccaacttcaacgagaagttcaagaaccgggtgaccctgaccaccgactcctccaccaccaccgcctacatggagctgaagtccctgcagtttgacgacaccgccgtgtactactgcgccaggagggactaccggttcgacatgggcttcgactactggggccagggcacaaccgtgaccgtgtccagcggaggtggcggatctggagggggtggtagcggtggaggcgggagtgagatcgtgctgacccagtcccctgctacactgtccctgtcccccggcgagagggctacactgagctgcagggcctccaagggcgtgtccacctccggctactcctacctgcactggtaccagcagaagcctggacaggctcccaggctgctgatctacctggcctcctacctggagtccggcgtgcctgctaggttttccggcagcggcagcggcaccgatttcaccctgaccatctcctccctggagcccgaggacttcgccgtgtactactgccagcactccagggatctgcctctgaccttcggcggcggcaccaaggtggagatcaagaccacaagtagaacggacattataacaacaggcacttttggtcagcagtccggagcgatatatgtgggcaattacaggatagtcaataggcacctggcaacacactttgactggcaaaattgcatatgggaggattacaacagggacctcctagtatgcactacaacggcgcacggttgtgacaatattgcaagatgccagtgcacagcaggtgtgtattattgtgcttctaggaacaagcattacccggtgcattttgaaggaccaggcttggtggaggtccaggaaagtgaatattatccaaaaaggtatcaatcacacgtgcttcttgccgcagggttttctgagccgggggattgtggtggtattctcaggtgtgaacatggtgttataggcatcgtgaccatgggtggtgagggtgttgtcggctttgccgatgtgcgcgatctcttatggctggaagacgacgcaatggaacagggagtcagggactacgtggaacagcttggaaacgcctttggttctgggtttaccaaccagatttgcgagcaagtcaatctgttaaaagagtcactaataggccaagactccatcttggaaaaatcactcaaggcattagtgaaaatcatatcagcactggttatcgtggtgagaaaccacgatgacctcataacagtcactgccaccttagccctgattggctgtaccacttcaccgtggcggtggctaaagaaaaaagtgactcaatactatgggattcccatggccgagagacagaacaatggctggctcaagaaattcacagaaatgaccaatgcatgcaagggcatggaatggatcgccatcaaaatccaaaagttcattgagtggctcaaaattaagatcttgccagaagtaaaggaaaaacacgagtttcttaccagacttaagcaactcccactcttagaaagccaaattgccaccatcgagcagagtgcaccatcacagagtgatcaggaacaactgttctccaatatacagtacttcgcgcactattgcagaaagtacgccccactgtacgcagctgaggcaaaaagagtgttctctctagaaaagaaaatgagcaactacatacagttcaagtccaaatgccgtattgaaccagtctgtttattgctccatggtagccctggagccggaaaatccgtggctaccaatttgattggacgctcacttgcagagaagctcaacagttcagtgtattcattgccaccagacccagaccattttgacggctataaacaacaagctgtcgtaatcatggatgacctgtgccagaacccagatgggaaagacgtgtccttgttctgccagatggtttcaagtgtagactttgttccaccaatggcagcacttgaggagaaaggcatactcttcacatcgcctttcgtgctcgcctctaccaacgcaggttccatcaacgctcccactgtgtcagacagcagagcccttgctagaaggttccacttcgacttgaacattgaggtcatctcaatgtacagccagaatggcaaaatcaacatgccaatgtcagtgaaggcttgtgatgatgagtgttgtccagttaatttcaagaggtgttgtccattagtgtgcggtaaggccattcagttcattgacagaagaacacaaatgaggtattcactagacatgcttgtaactgaaatgttcagggagtacaaccacagacatagtgtgggggcaacactcgaggcactcttccaaggcccacccgtgtacaaagagatcaagatcagtgttgcgccagaaacccctcccccaccggcaatagcagatctgctgaaatctgtggacagtgaagcagttagagagtactgcaaggaaaagggatggttggtgcctgagatcaattccactctacaaattgaaaagcacgtgagcagggccttcatttgcctgcaagcgctcaccacgtttgtttccgtggcggggataatttacatcatatacaaattgtttgctggcttccagggggcctacaccggtatgccaaaccaaaaacctaaagtaccaactttgaggcaggctaaggtgcagggaccggcattcgaattcgctgtggcaatgatgaaaagaaatgctagcacagtgaagaccgagtacggtgagttcacaatgcttggaatctatgacaggtgggcagtgctacctcgtcacgctagaccagggcctaccatcctaatgaatgaccaggaagtgggtgtggtggatgccaaggagttagtggacaaggatggcaccaatttagaattaacactcttgaagctcaatagaaatgagaaattcagagacatccgaggcttcttaacacgtgaggaggccgaagtcaacgaggctgtgcttgctattaacaccagcaagttcccaaatatgtacattccagttgggcaagtaacagattatggcttcctcaacttgggtggcacaccaactaagcgcatgctgatgtacaacttcccaacacgtgcaggccaatgtgggggagttctaatgtctacaggtaaagtgctcggaatacacgtcggtggtaatggacaccaagggttttcggcagccctgttacgccactatttcaatgacgagcagggagagattgagttcatcgagagctctaaggatgcaggctttccagtcattaacacacctagcaaaacaaagctcgagcccagtgttttccatcacgtgtttgagggaaacaaagaaccagcagtgttgaggaatggagacccaagactgaaagccaattttgaggaagccattttctccaagtacatcgggaatgtgaacacacatgtagacgagtacatgatggaagccgtcgaccattatgcaggacagttagctactttagatatcagtacagaacctatgaagcttgaggatgctgtctacggtactgagggcttggaagctctggacttgaccactagtgctgggtatccctatgtcgcccttggtataaaaaagagagacatcttatccaaaaagtctaaggacctgtctaaactgagagagtgtatggacaagtatgggttaaacctccctatggtcacctatgtgaaggatgaattgaggtcagcagagaaagtggctaagggcaaatccagactaatcgaggcgtccagtttaaatgactcagtggcaatgagacagactttcggtaacctgtataagacattccatctgaacccgggcatagtgactggcagtgcagttggatgcgatcccgacttgttctggagcaagattccagtcatgctcgatggtcacctcatcgcctttgactacagtggatatgatgccagcctgagtccggtgtggtttgcttgcttgaaactgttgttggaaaagctggggtactcacacaaggaaactaattacatagattacttgtgcaactcccaccatttgtacagagacaagcactactttgtgaggggaggaatgccatccgggtgttctggaaccagcattttcaattcaatgattaataacataatcatcagaacactgatgttgaaggtgtacaaagggatagatctagaccaatttaggatgattgcatatggtgacgacgtcattgcttcgtacccgtatccaattgatgcgtcattgctggctgaggctggcaaggggtatggtctgattatgacaccagcagacaaaggagagtgtttcaatgaagtaacgtggaccaacgtcactttcctgaaaagatacttcagagcggatgagcaatatcccttcctcgttcacccagtgatgccaatgaaggacattcatgagtcaattaggtggactaaagaccccaaaaacacccaagatcacgtgcgatccttgtgcttattggcctggcacaatggagagcaagaatatgagaattttgtatccaaaatcagaagcgtcccagttgggcgctgcttgacattgcctgcgttctcgactctgcgcaggaagtggttggattccttctaaaattgaagtacaatgtgatttaactcaattggcttaaccctaccacacttaccgaactagataacggtgtggtaggggtaaattc

SEQ ID NO: 12 - RNA, 7371 nt, Artificial Sequence
  Genomic sequence of CVB1-WT
uccuguggguugaucccacccacagggcccacugggcgcuagcacacugguauuccgguaccuuugugcgccuguuuuauacacccuucccaaguguaacuuagaagcuuaucacacacggucaacaggugacucaguaugccaacugggucuugaucaagcacuucuguuuccccggacugaguaucaauaagcugcucacgcggcugaaggagaaaacguucguuaaccggccaauuacuucgagaaaccuaguaacaccauggagguugcgcaguguuucgcuccacacaaccccaguguagaucaggccgaugagucaccgcacuccucacgggcgaccguggcgguggcugcgcuggcggccugcccaugggauaacccaugggacgcuucaauacugacauggugcgaagagucuauugagcuaauugguaguccuccggccccugaaugcggcuaauccuaacugcggagcagauacccacacgccagugggcagucugucguaaugggcaacucugcagcggaaccgacuacuuuggguguccguguuuccuuuuauuuuuauacuggcugcuuauggugacaauugagagauuguuaccauauagcuauuggauuggccauccagugacaaacagagcgauuauuuauuuauuuguugguuauaucccauuaagccaaaaggcccuaguuacucucaacuuuauacuauugcuuaauacaacgaaaugggagcucaggugucuacacaaaagacgggugcacaugagacuggcuugaaugccagcggcaauucugucauccauuacaccaacaucaauuacuacaaggaugcugcauccaacuccgcaaauaggcaagacuucacacaagacccagguaaguucacugaaccuguaaaggacaucauggugaaaacuaugccugcguugaacucacccucugcugaggaaugcggcuacagugacagagugcguuccaucacauuagguaauuccacuaucaccacccaagagugugccaacguggucgucggguacggaguguggccagaguaucuaaaagacaacgaggccacagccgaagaucagcccacacaaccugauguggccaccugccgcuucuauacuuuagaaucagugcaguggaugaaaaauucggccggguggugguggaagcugccugaugcacugucacagaugggcuuguuuggucaaaacaugcaguaccacuacuugggaagaacaggguauacuauacacguacaaugcaaugccucaaaguuccaucaaggaugccugcuuguagugugugugccugaagcugagauggggugcucuaacuugaacaacacgcccgaauucagugaguuauccggaggagacagugccagaauguucaccgauacacaggucggggagucaaacgccaagaaaguacagacagcgguguggaacgccggaaugggcguuggaguggguaaucucacuauuuucccucaccaauggaucaaccugaggaccaacaacagugcaacgcuagugaugccuuacauaaacagcguccccauggauaacauguucaggcacaauaacuuaacacuaaugauuaucccauuuguaccgcuuaauuacagcgagggauccucaccguacgugcccauaacagucaccaucgcuccuaugugugcagaguacaacgggcuacggcuggccagcaaccagggucuaccaguuaugacaacaccugggagcacgcaauuucuaacuucagaugacuuccagucaccgucagcgaugccacaguuugaugucaccccagagaugcaaauaccaggccgcguaaauaauuugauggagauugccgagguggacucaguggugccugugaauaacacagaggacaacgugaguagccuuaaggcauaccagauuccaguacaguccaacagugacaaugguaagcagguuuuugguuuucccuuacaaccgggugccaacaacguucugaauagaacccucuugggugaaauucuaaacuauuacacccauuggaguggcagcauaaaacuuacauucauguucugugggucugccauggccacuggcaaguuccuucuggcauacucaccaccuggagcaggagucccgaaaaaccgaaaagaugcuaugcugggcacccacgucauaugggauguuggguuacaaucaagcugcguguugugcguaccguggauuagccaaacacacuacagauacgugguugaggaugaauacacagcagccggauauguuacaugcugguaucaaacaaauaucguggugccagcugaugugcaaagcucaugugacaucuugugcuuuguuucagccugcaacgauuucucugugcggaugcugaaagauacgcccuuuauaagacaggacacauuuuaucacggcccaguggaggagucuguggaucgugcaguggcacguguggcggacacgauuaguucacggcccacuaacucggagucaauuccagcgcucaccgccgccgagacugggcauaccucccaaguugugccuagcgacaccaugcaaacaagacaugugaaaaacuaccacucacgguccgaaucguccauugaaaauuuccuaugccggucugccuguguauacuaugccacauacaccaauaacucaaaaaaagagggguuugcagaguggguuaucaacacuagacagguggcacagcuaagaagaaaguuggagcucuucacauaucuaagguuugacuuagaacuaacauuuguuauaacgagcgcacaacaacccaguaccgccuccaguguagacgcucccgugcaaacccaucagauuauguaugugccuccagguggcccuguaccaacaaagguuaaagauuaugcauggcaaacuuccacaaaccccagcguuuucuggacagaaggaaacgccccaccaaggauguccaucccauucauuagcauugguaaugcguauaguuguuuuuaugauggguggacgcaguuuucaagaaacggggucuauggcaucaauacccucaacaacaugggcacguuguauaugaggcaugucaaugaagcggggcaggguccaaucaaaaguacuguuagaauauauuucaaacccaagcacgugaaggcgugggugccacguccgccaagauugugucaauaugagaagcagaaaaaugucaauuuuagcccuauaggaguaaccacaaguagaacggacauuauaacaacaggcacuuuuggucagcaguccggagcgauauaugugggcaauuacaggauagucaauaggcaccuggcaacacacuuugacuggcaaaauugcauaugggaggauuacaacagggaccuccuaguaugcacuacaacggcgcacgguugugacaauauugcaagaugccagugcacagcagguguguauuauugugcuucuaggaacaagcauuacccggugcauuuugaaggaccaggcuugguggagguccaggaaagugaauauuauccaaaaagguaucaaucacacgugcuucuugccgcaggguuuucugagccgggggauuguggugguauucucaggugugaacaugguguuauaggcaucgugaccauggguggugaggguguugucggcuuugccgaugugcgcgaucucuuauggcuggaagacgacgcaauggaacagggagucagggacuacguggaacagcuuggaaacgccuuugguucuggguuuaccaaccagauuugcgagcaagucaaucuguuaaaagagucacuaauaggccaagacuccaucuuggaaaaaucacucaaggcauuagugaaaaucauaucagcacugguuaucguggugagaaaccacgaugaccucauaacagucacugccaccuuagcccugauuggcuguaccacuucaccguggcgguggcuaaagaaaaaagugacucaauacuaugggauucccauggccgagagacagaacaauggcuggcucaagaaauucacagaaaugaccaaugcaugcaagggcauggaauggaucgccaucaaaauccaaaaguucauugaguggcucaaaauuaagaucuugccagaaguaaaggaaaaacacgaguuucuuaccagacuuaagcaacucccacucuuagaaagccaaauugccaccaucgagcagagugcaccaucacagagugaucaggaacaacuguucuccaauauacaguacuucgcgcacuauugcagaaaguacgccccacuguacgcagcugaggcaaaaagaguguucucucuagaaaagaaaaugagcaacuacauacaguucaaguccaaaugccguauugaaccagucuguuuauugcuccaugguagcccuggagccggaaaauccguggcuaccaauuugauuggacgcucacuugcagagaagcucaacaguucaguguauucauugccaccagacccagaccauuuugacggcuauaaacaacaagcugucguaaucauggaugaccugugccagaacccagaugggaaagacguguccuuguucugccagaugguuucaaguguagacuuuguuccaccaauggcagcacuugaggagaaaggcauacucuucacaucgccuuucgugcucgccucuaccaacgcagguuccaucaacgcucccacugugucagacagcagagcccuugcuagaagguuccacuucgacuugaacauugaggucaucucaauguacagccagaauggcaaaaucaacaugccaaugucagugaaggcuugugaugaugaguguuguccaguuaauuucaagagguguuguccauuagugugcgguaaggccauucaguucauugacagaagaacacaaaugagguauucacuagacaugcuuguaacugaaauguucagggaguacaaccacagacauagugugggggcaacacucgaggcacucuuccaaggcccacccguguacaaagagaucaagaucaguguugcgccagaaaccccucccccaccggcaauagcagaucugcugaaaucuguggacagugaagcaguuagagaguacugcaaggaaaagggaugguuggugccugagaucaauuccacucuacaaauugaaaagcacgugagcagggccuucauuugccugcaagcgcucaccacguuuguuuccguggcggggauaauuuacaucauauacaaauuguuugcuggcuuccagggggccuacaccgguaugccaaaccaaaaaccuaaaguaccaacuuugaggcaggcuaaggugcagggaccggcauucgaauucgcuguggcaaugaugaaaagaaaugcuagcacagugaagaccgaguacggugaguucacaaugcuuggaaucuaugacaggugggcagugcuaccucgucacgcuagaccagggccuaccauccuaaugaaugaccaggaagugggugugguggaugccaaggaguuaguggacaaggauggcaccaauuuagaauuaacacucuugaagcucaauagaaaugagaaauucagagacauccgaggcuucuuaacacgugaggaggccgaagucaacgaggcugugcuugcuauuaacaccagcaaguucccaaauauguacauuccaguugggcaaguaacagauuauggcuuccucaacuuggguggcacaccaacuaagcgcaugcugauguacaacuucccaacacgugcaggccaaugugggggaguucuaaugucuacagguaaagugcucggaauacacgucggugguaauggacaccaaggguuuucggcagcccuguuacgccacuauuucaaugacgagcagggagagauugaguucaucgagagcucuaaggaugcaggcuuuccagucauuaacacaccuagcaaaacaaagcucgagcccaguguuuuccaucacguguuugagggaaacaaagaaccagcaguguugaggaauggagacccaagacugaaagccaauuuugaggaagccauuuucuccaaguacaucgggaaugugaacacacauguagacgaguacaugauggaagccgucgaccauuaugcaggacaguuagcuacuuuagauaucaguacagaaccuaugaagcuugaggaugcugucuacgguacugagggcuuggaagcucuggacuugaccacuagugcuggguaucccuaugucgcccuugguauaaaaaagagagacaucuuauccaaaaagucuaaggaccugucuaaacugagagaguguauggacaaguauggguuaaaccucccuauggucaccuaugugaaggaugaauugaggucagcagagaaaguggcuaagggcaaauccagacuaaucgaggcguccaguuuaaaugacucaguggcaaugagacagacuuucgguaaccuguauaagacauuccaucugaacccgggcauagugacuggcagugcaguuggaugcgaucccgacuuguucuggagcaagauuccagucaugcucgauggucaccucaucgccuuugacuacaguggauaugaugccagccugaguccggugugguuugcuugcuugaaacuguuguuggaaaagcugggguacucacacaaggaaacuaauuacauagauuacuugugcaacucccaccauuuguacagagacaagcacuacuuugugaggggaggaaugccauccggguguucuggaaccagcauuuucaauucaaugauuaauaacauaaucaucagaacacugauguugaagguguacaaagggauagaucuagaccaauuuaggaugauugcauauggugacgacgucauugcuucguacccguauccaauugaugcgucauugcuggcugaggcuggcaagggguauggucugauuaugacaccagcagacaaaggagaguguuucaaugaaguaacguggaccaacgucacuuuccugaaaagauacuucagagcggaugagcaauaucccuuccucguucacccagugaugccaaugaaggacauucaugagucaauuagguggacuaaagaccccaaaaacacccaagaucacgugcgauccuugugcuuauuggccuggcacaauggagagcaagaauaugagaauuuuguauccaaaaucagaagcgucccaguugggcgcugcuugacauugccugcguucucgacucugcgcaggaagugguuggauuccuucuaaaauugaaguacaaugugauuuaacucaauuggcuuaacccuaccacacuuaccgaacuagauaacggugugguagggguaaauuc

SEQ ID NO: 13 - RNA, 7251 nt, Artificial Sequence
  Genomic sequence of CVB1-HRV2
uccuguggguugaucccacccacagggcccacugggcgcuagcacacugguauuccgguaccuuugugcgccuguuuuauacacccuucccaaguguaacuuagaagaacuuagaaguuuuucacaaagaccaauagccgguaaucagccagauuacugaaggucaagcacuucuguuuccccggucaauguugauaugcuccaacagggcaaaaacaacugcgaucguuaaccgcaaagcgccuacgcaaagcuuaguagcaucuuugaaaucguuuggcuggucgauccgccauuuccccugguagaccuggcagaugaggcuagaaauaccccacuggcgacaguguucuagccugcguggcugccugcacacccuaugggugugaagccaaacaauggacaaggugugaagagccccgugugcucgcuuugaguccuccggccccugaauguggcuaaccuuaacccugcagcuagagcacguaacccaauguguaucuagucguaaugagcaauugcgggaugggaccaacuacuuuggguguccguguuucacuuuuuccuuuauauuugcuuauggugacaauauauacaauauauauauuggcaccaugggagcucaggugucuacacaaaagacgggugcacaugagacuggcuugaaugccagcggcaauucugucauccauuacaccaacaucaauuacuacaaggaugcugcauccaacuccgcaaauaggcaagacuucacacaagacccagguaaguucacugaaccuguaaaggacaucauggugaaaacuaugccugcguugaacucacccucugcugaggaaugcggcuacagugacagagugcguuccaucacauuagguaauuccacuaucaccacccaagagugugccaacguggucgucggguacggaguguggccagaguaucuaaaagacaacgaggccacagccgaagaucagcccacacaaccugauguggccaccugccgcuucuauacuuuagaaucagugcaguggaugaaaaauucggccggguggugguggaagcugccugaugcacugucacagaugggcuuguuuggucaaaacaugcaguaccacuacuugggaagaacaggguauacuauacacguacaaugcaaugccucaaaguuccaucaaggaugccugcuuguagugugugugccugaagcugagauggggugcucuaacuugaacaacacgcccgaauucagugaguuauccggaggagacagugccagaauguucaccgauacacaggucggggagucaaacgccaagaaaguacagacagcgguguggaacgccggaaugggcguuggaguggguaaucucacuauuuucccucaccaauggaucaaccugaggaccaacaacagugcaacgcuagugaugccuuacauaaacagcguccccauggauaacauguucaggcacaauaacuuaacacuaaugauuaucccauuuguaccgcuuaauuacagcgagggauccucaccguacgugcccauaacagucaccaucgcuccuaugugugcagaguacaacgggcuacggcuggccagcaaccagggucuaccaguuaugacaacaccugggagcacgcaauuucuaacuucagaugacuuccagucaccgucagcgaugccacaguuugaugucaccccagagaugcaaauaccaggccgcguaaauaauuugauggagauugccgagguggacucaguggugccugugaauaacacagaggacaacgugaguagccuuaaggcauaccagauuccaguacaguccaacagugacaaugguaagcagguuuuugguuuucccuuacaaccgggugccaacaacguucugaauagaacccucuugggugaaauucuaaacuauuacacccauuggaguggcagcauaaaacuuacauucauguucugugggucugccauggccacuggcaaguuccuucuggcauacucaccaccuggagcaggagucccgaaaaaccgaaaagaugcuaugcugggcacccacgucauaugggauguuggguuacaaucaagcugcguguugugcguaccguggauuagccaaacacacuacagauacgugguugaggaugaauacacagcagccggauauguuacaugcugguaucaaacaaauaucguggugccagcugaugugcaaagcucaugugacaucuugugcuuuguuucagccugcaacgauuucucugugcggaugcugaaagauacgcccuuuauaagacaggacacauuuuaucacggcccaguggaggagucuguggaucgugcaguggcacguguggcggacacgauuaguucacggcccacuaacucggagucaauuccagcgcucaccgccgccgagacugggcauaccucccaaguugugccuagcgacaccaugcaaacaagacaugugaaaaacuaccacucacgguccgaaucguccauugaaaauuuccuaugccggucugccuguguauacuaugccacauacaccaauaacucaaaaaaagagggguuugcagaguggguuaucaacacuagacagguggcacagcuaagaagaaaguuggagcucuucacauaucuaagguuugacuuagaacuaacauuuguuauaacgagcgcacaacaacccaguaccgccuccaguguagacgcucccgugcaaacccaucagauuauguaugugccuccagguggcccuguaccaacaaagguuaaagauuaugcauggcaaacuuccacaaaccccagcguuuucuggacagaaggaaacgccccaccaaggauguccaucccauucauuagcauugguaaugcguauaguuguuuuuaugauggguggacgcaguuuucaagaaacggggucuauggcaucaauacccucaacaacaugggcacguuguauaugaggcaugucaaugaagcggggcaggguccaaucaaaaguacuguuagaauauauuucaaacccaagcacgugaaggcgugggugccacguccgccaagauugugucaauaugagaagcagaaaaaugucaauuuuagcccuauaggaguaaccacaaguagaacggacauuauaacaacaggcacuuuuggucagcaguccggagcgauauaugugggcaauuacaggauagucaauaggcaccuggcaacacacuuugacuggcaaaauugcauaugggaggauuacaacagggaccuccuaguaugcacuacaacggcgcacgguugugacaauauugcaagaugccagugcacagcagguguguauuauugugcuucuaggaacaagcauuacccggugcauuuugaaggaccaggcuugguggagguccaggaaagugaauauuauccaaaaagguaucaaucacacgugcuucuugccgcaggguuuucugagccgggggauuguggugguauucucaggugugaacaugguguuauaggcaucgugaccauggguggugaggguguugucggcuuugccgaugugcgcgaucucuuauggcuggaagacgacgcaauggaacagggagucagggacuacguggaacagcuuggaaacgccuuugguucuggguuuaccaaccagauuugcgagcaagucaaucuguuaaaagagucacuaauaggccaagacuccaucuuggaaaaaucacucaaggcauuagugaaaaucauaucagcacugguuaucguggugagaaaccacgaugaccucauaacagucacugccaccuuagcccugauuggcuguaccacuucaccguggcgguggcuaaagaaaaaagugacucaauacuaugggauucccauggccgagagacagaacaauggcuggcucaagaaauucacagaaaugaccaaugcaugcaagggcauggaauggaucgccaucaaaauccaaaaguucauugaguggcucaaaauuaagaucuugccagaaguaaaggaaaaacacgaguuucuuaccagacuuaagcaacucccacucuuagaaagccaaauugccaccaucgagcagagugcaccaucacagagugaucaggaacaacuguucuccaauauacaguacuucgcgcacuauugcagaaaguacgccccacuguacgcagcugaggcaaaaagaguguucucucuagaaaagaaaaugagcaacuacauacaguucaaguccaaaugccguauugaaccagucuguuuauugcuccaugguagcccuggagccggaaaauccguggcuaccaauuugauuggacgcucacuugcagagaagcucaacaguucaguguauucauugccaccagacccagaccauuuugacggcuauaaacaacaagcugucguaaucauggaugaccugugccagaacccagaugggaaagacguguccuuguucugccagaugguuucaaguguagacuuuguuccaccaauggcagcacuugaggagaaaggcauacucuucacaucgccuuucgugcucgccucuaccaacgcagguuccaucaacgcucccacugugucagacagcagagcccuugcuagaagguuccacuucgacuugaacauugaggucaucucaauguacagccagaauggcaaaaucaacaugccaaugucagugaaggcuugugaugaugaguguuguccaguuaauuucaagagguguuguccauuagugugcgguaaggccauucaguucauugacagaagaacacaaaugagguauucacuagacaugcuuguaacugaaauguucagggaguacaaccacagacauagugugggggcaacacucgaggcacucuuccaaggcccacccguguacaaagagaucaagaucaguguugcgccagaaaccccucccccaccggcaauagcagaucugcugaaaucuguggacagugaagcaguuagagaguacugcaaggaaaagggaugguuggugccugagaucaauuccacucuacaaauugaaaagcacgugagcagggccuucauuugccugcaagcgcucaccacguuuguuuccguggcggggauaauuuacaucauauacaaauuguuugcuggcuuccagggggccuacaccgguaugccaaaccaaaaaccuaaaguaccaacuuugaggcaggcuaaggugcagggaccggcauucgaauucgcuguggcaaugaugaaaagaaaugcuagcacagugaagaccgaguacggugaguucacaaugcuuggaaucuaugacaggugggcagugcuaccucgucacgcuagaccagggccuaccauccuaaugaaugaccaggaagugggugugguggaugccaaggaguuaguggacaaggauggcaccaauuuagaauuaacacucuugaagcucaauagaaaugagaaauucagagacauccgaggcuucuuaacacgugaggaggccgaagucaacgaggcugugcuugcuauuaacaccagcaaguucccaaauauguacauuccaguugggcaaguaacagauuauggcuuccucaacuuggguggcacaccaacuaagcgcaugcugauguacaacuucccaacacgugcaggccaaugugggggaguucuaaugucuacagguaaagugcucggaauacacgucggugguaauggacaccaaggguuuucggcagcccuguuacgccacuauuucaaugacgagcagggagagauugaguucaucgagagcucuaaggaugcaggcuuuccagucauuaacacaccuagcaaaacaaagcucgagcccaguguuuuccaucacguguuugagggaaacaaagaaccagcaguguugaggaauggagacccaagacugaaagccaauuuugaggaagccauuuucuccaaguacaucgggaaugugaacacacauguagacgaguacaugauggaagccgucgaccauuaugcaggacaguuagcuacuuuagauaucaguacagaaccuaugaagcuugaggaugcugucuacgguacugagggcuuggaagcucuggacuugaccacuagugcuggguaucccuaugucgcccuugguauaaaaaagagagacaucuuauccaaaaagucuaaggaccugucuaaacugagagaguguauggacaaguauggguuaaaccucccuauggucaccuaugugaaggaugaauugaggucagcagagaaaguggcuaagggcaaauccagacuaaucgaggcguccaguuuaaaugacucaguggcaaugagacagacuuucgguaaccuguauaagacauuccaucugaacccgggcauagugacuggcagugcaguuggaugcgaucccgacuuguucuggagcaagauuccagucaugcucgauggucaccucaucgccuuugacuacaguggauaugaugccagccugaguccggugugguuugcuugcuugaaacuguuguuggaaaagcugggguacucacacaaggaaacuaauuacauagauuacuugugcaacucccaccauuuguacagagacaagcacuacuuugugaggggaggaaugccauccggguguucuggaaccagcauuuucaauucaaugauuaauaacauaaucaucagaacacugauguugaagguguacaaagggauagaucuagaccaauuuaggaugauugcauauggugacgacgucauugcuucguacccguauccaauugaugcgucauugcuggcugaggcuggcaagggguauggucugauuaugacaccagcagacaaaggagaguguuucaaugaaguaacguggaccaacgucacuuuccugaaaagauacuucagagcggaugagcaauaucccuuccucguucacccagugaugccaaugaaggacauucaugagucaauuagguggacuaaagaccccaaaaacacccaagaucacgugcgauccuugugcuuauuggccuggcacaauggagagcaagaauaugagaauuuuguauccaaaaucagaagcgucccaguugggcgcugcuugacauugccugcguucucgacucugcgcaggaagugguuggauuccuucuaaaauugaaguacaaugugauuuaacucaauuggcuuaacccuaccacacuuaccgaacuagauaacggugugguagggguaaauuc

SEQ ID NO: 14 - RNA, 7473 nt, Artificial Sequence
  Genomic sequence of CVB1-miR133&206T
uccuguggguugaucccacccacagggcccacugggcgcuagcacacugguauuccgguaccuuugugcgccuguuuuauacacccuucccaaguguaacuuagaagcuuaucacacacggucaacaggugacucaguaugccaacugggucuugaucaagcacuucuguuuccccggacugaguaucaauaagcugcucacgcggcugaaggagaaaacguucguuaaccggccaauuacuucgagaaaccuaguaacaccauggagguugcgcaguguuucgcuccacacaaccccaguguagaucaggccgaugagucaccgcacuccucacgggcgaccguggcgguggcugcgcuggcggccugcccaugggauaacccaugggacgcuucaauacugacauggugcgaagagucuauugagcuaauugguaguccuccggccccugaaugcggcuaauccuaacugcggagcagauacccacacgccagugggcagucugucguaaugggcaacucugcagcggaaccgacuacuuuggguguccguguuuccuuuuauuuuuauacuggcugcuuauggugacaauugagagauuguuaccauauagcuauuggauuggccauccagugacaaacagagcgauuauuuauuuauuuguugguuauaucccauuaagccaaaaggcccuaguuacucucaacuuuauacuauugcuuaauacaacgaaaugggagcucaggugucuacacaaaagacgggugcacaugagacuggcuugaaugccagcggcaauucugucauccauuacaccaacaucaauuacuacaaggaugcugcauccaacuccgcaaauaggcaagacuucacacaagacccagguaaguucacugaaccuguaaaggacaucauggugaaaacuaugccugcguugaacucacccucugcugaggaaugcggcuacagugacagagugcguuccaucacauuagguaauuccacuaucaccacccaagagugugccaacguggucgucggguacggaguguggccagaguaucuaaaagacaacgaggccacagccgaagaucagcccacacaaccugauguggccaccugccgcuucuauacuuuagaaucagugcaguggaugaaaaauucggccggguggugguggaagcugccugaugcacugucacagaugggcuuguuuggucaaaacaugcaguaccacuacuugggaagaacaggguauacuauacacguacaaugcaaugccucaaaguuccaucaaggaugccugcuuguagugugugugccugaagcugagauggggugcucuaacuugaacaacacgcccgaauucagugaguuauccggaggagacagugccagaauguucaccgauacacaggucggggagucaaacgccaagaaaguacagacagcgguguggaacgccggaaugggcguuggaguggguaaucucacuauuuucccucaccaauggaucaaccugaggaccaacaacagugcaacgcuagugaugccuuacauaaacagcguccccauggauaacauguucaggcacaauaacuuaacacuaaugauuaucccauuuguaccgcuuaauuacagcgagggauccucaccguacgugcccauaacagucaccaucgcuccuaugugugcagaguacaacgggcuacggcuggccagcaaccagggucuaccaguuaugacaacaccugggagcacgcaauuucuaacuucagaugacuuccagucaccgucagcgaugccacaguuugaugucaccccagagaugcaaauaccaggccgcguaaauaauuugauggagauugccgagguggacucaguggugccugugaauaacacagaggacaacgugaguagccuuaaggcauaccagauuccaguacaguccaacagugacaaugguaagcagguuuuugguuuucccuuacaaccgggugccaacaacguucugaauagaacccucuugggugaaauucuaaacuauuacacccauuggaguggcagcauaaaacuuacauucauguucugugggucugccauggccacuggcaaguuccuucuggcauacucaccaccuggagcaggagucccgaaaaaccgaaaagaugcuaugcugggcacccacgucauaugggauguuggguuacaaucaagcugcguguugugcguaccguggauuagccaaacacacuacagauacgugguugaggaugaauacacagcagccggauauguuacaugcugguaucaaacaaauaucguggugccagcugaugugcaaagcucaugugacaucuugugcuuuguuucagccugcaacgauuucucugugcggaugcugaaagauacgcccuuuauaagacaggacacauuuuaucacggcccaguggaggagucuguggaucgugcaguggcacguguggcggacacgauuaguucacggcccacuaacucggagucaauuccagcgcucaccgccgccgagacugggcauaccucccaaguugugccuagcgacaccaugcaaacaagacaugugaaaaacuaccacucacgguccgaaucguccauugaaaauuuccuaugccggucugccuguguauacuaugccacauacaccaauaacucaaaaaaagagggguuugcagaguggguuaucaacacuagacagguggcacagcuaagaagaaaguuggagcucuucacauaucuaagguuugacuuagaacuaacauuuguuauaacgagcgcacaacaacccaguaccgccuccaguguagacgcucccgugcaaacccaucagauuauguaugugccuccagguggcccuguaccaacaaagguuaaagauuaugcauggcaaacuuccacaaaccccagcguuuucuggacagaaggaaacgccccaccaaggauguccaucccauucauuagcauugguaaugcguauaguuguuuuuaugauggguggacgcaguuuucaagaaacggggucuauggcaucaauacccucaacaacaugggcacguuguauaugaggcaugucaaugaagcggggcaggguccaaucaaaaguacuguuagaauauauuucaaacccaagcacgugaaggcgugggugccacguccgccaagauugugucaauaugagaagcagaaaaaugucaauuuuagcccuauaggaguaaccacaaguagaacggacauuauaacaacaggcacuuuuggucagcaguccggagcgauauaugugggcaauuacaggauagucaauaggcaccuggcaacacacuuugacuggcaaaauugcauaugggaggauuacaacagggaccuccuaguaugcacuacaacggcgcacgguugugacaauauugcaagaugccagugcacagcagguguguauuauugugcuucuaggaacaagcauuacccggugcauuuugaaggaccaggcuugguggagguccaggaaagugaauauuauccaaaaagguaucaaucacacgugcuucuugccgcaggguuuucugagccgggggauuguggugguauucucaggugugaacaugguguuauaggcaucgugaccauggguggugaggguguugucggcuuugccgaugugcgcgaucucuuauggcuggaagacgacgcaauggaacagggagucagggacuacguggaacagcuuggaaacgccuuugguucuggguuuaccaaccagauuugcgagcaagucaaucuguuaaaagagucacuaauaggccaagacuccaucuuggaaaaaucacucaaggcauuagugaaaaucauaucagcacugguuaucguggugagaaaccacgaugaccucauaacagucacugccaccuuagcccugauuggcuguaccacuucaccguggcgguggcuaaagaaaaaagugacucaauacuaugggauucccauggccgagagacagaacaauggcuggcucaagaaauucacagaaaugaccaaugcaugcaagggcauggaauggaucgccaucaaaauccaaaaguucauugaguggcucaaaauuaagaucuugccagaaguaaaggaaaaacacgaguuucuuaccagacuuaagcaacucccacucuuagaaagccaaauugccaccaucgagcagagugcaccaucacagagugaucaggaacaacuguucuccaauauacaguacuucgcgcacuauugcagaaaguacgccccacuguacgcagcugaggcaaaaagaguguucucucuagaaaagaaaaugagcaacuacauacaguucaaguccaaaugccguauugaaccagucuguuuauugcuccaugguagcccuggagccggaaaauccguggcuaccaauuugauuggacgcucacuugcagagaagcucaacaguucaguguauucauugccaccagacccagaccauuuugacggcuauaaacaacaagcugucguaaucauggaugaccugugccagaacccagaugggaaagacguguccuuguucugccagaugguuucaaguguagacuuuguuccaccaauggcagcacuugaggagaaaggcauacucuucacaucgccuuucgugcucgccucuaccaacgcagguuccaucaacgcucccacugugucagacagcagagcccuugcuagaagguuccacuucgacuugaacauugaggucaucucaauguacagccagaauggcaaaaucaacaugccaaugucagugaaggcuugugaugaugaguguuguccaguuaauuucaagagguguuguccauuagugugcgguaaggccauucaguucauugacagaagaacacaaaugagguauucacuagacaugcuuguaacugaaauguucagggaguacaaccacagacauagugugggggcaacacucgaggcacucuuccaaggcccacccguguacaaagagaucaagaucaguguugcgccagaaaccccucccccaccggcaauagcagaucugcugaaaucuguggacagugaagcaguuagagaguacugcaaggaaaagggaugguuggugccugagaucaauuccacucuacaaauugaaaagcacgugagcagggccuucauuugccugcaagcgcucaccacguuuguuuccguggcggggauaauuuacaucauauacaaauuguuugcuggcuuccagggggccuacaccgguaugccaaaccaaaaaccuaaaguaccaacuuugaggcaggcuaaggugcagggaccggcauucgaauucgcuguggcaaugaugaaaagaaaugcuagcacagugaagaccgaguacggugaguucacaaugcuuggaaucuaugacaggugggcagugcuaccucgucacgcuagaccagggccuaccauccuaaugaaugaccaggaagugggugugguggaugccaaggaguuaguggacaaggauggcaccaauuuagaauuaacacucuugaagcucaauagaaaugagaaauucagagacauccgaggcuucuuaacacgugaggaggccgaagucaacgaggcugugcuugcuauuaacaccagcaaguucccaaauauguacauuccaguugggcaaguaacagauuauggcuuccucaacuuggguggcacaccaacuaagcgcaugcugauguacaacuucccaacacgugcaggccaaugugggggaguucuaaugucuacagguaaagugcucggaauacacgucggugguaauggacaccaaggguuuucggcagcccuguuacgccacuauuucaaugacgagcagggagagauugaguucaucgagagcucuaaggaugcaggcuuuccagucauuaacacaccuagcaaaacaaagcucgagcccaguguuuuccaucacguguuugagggaaacaaagaaccagcaguguugaggaauggagacccaagacugaaagccaauuuugaggaagccauuuucuccaaguacaucgggaaugugaacacacauguagacgaguacaugauggaagccgucgaccauuaugcaggacaguuagcuacuuuagauaucaguacagaaccuaugaagcuugaggaugcugucuacgguacugagggcuuggaagcucuggacuugaccacuagugcuggguaucccuaugucgcccuugguauaaaaaagagagacaucuuauccaaaaagucuaaggaccugucuaaacugagagaguguauggacaaguauggguuaaaccucccuauggucaccuaugugaaggaugaauugaggucagcagagaaaguggcuaagggcaaauccagacuaaucgaggcguccaguuuaaaugacucaguggcaaugagacagacuuucgguaaccuguauaagacauuccaucugaacccgggcauagugacuggcagugcaguuggaugcgaucccgacuuguucuggagcaagauuccagucaugcucgauggucaccucaucgccuuugacuacaguggauaugaugccagccugaguccggugugguuugcuugcuugaaacuguuguuggaaaagcugggguacucacacaaggaaacuaauuacauagauuacuugugcaacucccaccauuuguacagagacaagcacuacuuugugaggggaggaaugccauccggguguucuggaaccagcauuuucaauucaaugauuaauaacauaaucaucagaacacugauguugaagguguacaaagggauagaucuagaccaauuuaggaugauugcauauggugacgacgucauugcuucguacccguauccaauugaugcgucauugcuggcugaggcuggcaagggguauggucugauuaugacaccagcagacaaaggagaguguuucaaugaaguaacguggaccaacgucacuuuccugaaaagauacuucagagcggaugagcaauaucccuuccucguucacccagugaugccaaugaaggacauucaugagucaauuagguggacuaaagaccccaaaaacacccaagaucacgugcgauccuugugcuuauuggccuggcacaauggagagcaagaauaugagaauuuuguauccaaaaucagaagcgucccaguugggcgcugcuugacauugccugcguucucgacucugcgcaggaagugguuggauuccuucuaaaauacagcugguugaaggggaccaacgauacagcugguugaaggggaccaaaccgguccacacacuuccuuacauuccaucacccacacacuuccuuacauuccaugaaguacaaugugauuuaacucaauuggcuuaacccuaccacacuuaccgaacuagauaacggugugguagggguaaauuc

SEQ ID NO: 15 - RNA, 7848 nt, Artificial Sequence
  Genomic sequence of CVB1-GM-CSF
uccuguggguugaucccacccacagggcccacugggcgcuagcacacugguauuccgguaccuuugugcgccuguuuuauacacccuucccaaguguaacuuagaagcuuaucacacacggucaacaggugacucaguaugccaacugggucuugaucaagcacuucuguuuccccggacugaguaucaauaagcugcucacgcggcugaaggagaaaacguucguuaaccggccaauuacuucgagaaaccuaguaacaccauggagguugcgcaguguuucgcuccacacaaccccaguguagaucaggccgaugagucaccgcacuccucacgggcgaccguggcgguggcugcgcuggcggccugcccaugggauaacccaugggacgcuucaauacugacauggugcgaagagucuauugagcuaauugguaguccuccggccccugaaugcggcuaauccuaacugcggagcagauacccacacgccagugggcagucugucguaaugggcaacucugcagcggaaccgacuacuuuggguguccguguuuccuuuuauuuuuauacuggcugcuuauggugacaauugagagauuguuaccauauagcuauuggauuggccauccagugacaaacagagcgauuauuuauuuauuuguugguuauaucccauuaagccaaaaggcccuaguuacucucaacuuuauacuauugcuuaauacaacgaaaugggagcucaggugucuacacaaaagacgggugcacaugagacuggcuugaaugccagcggcaauucugucauccauuacaccaacaucaauuacuacaaggaugcugcauccaacuccgcaaauaggcaagacuucacacaagacccagguaaguucacugaaccuguaaaggacaucauggugaaaacuaugccugcguugaacucacccucugcugaggaaugcggcuacagugacagagugcguuccaucacauuagguaauuccacuaucaccacccaagagugugccaacguggucgucggguacggaguguggccagaguaucuaaaagacaacgaggccacagccgaagaucagcccacacaaccugauguggccaccugccgcuucuauacuuuagaaucagugcaguggaugaaaaauucggccggguggugguggaagcugccugaugcacugucacagaugggcuuguuuggucaaaacaugcaguaccacuacuugggaagaacaggguauacuauacacguacaaugcaaugccucaaaguuccaucaaggaugccugcuuguagugugugugccugaagcugagauggggugcucuaacuugaacaacacgcccgaauucagugaguuauccggaggagacagugccagaauguucaccgauacacaggucggggagucaaacgccaagaaaguacagacagcgguguggaacgccggaaugggcguuggaguggguaaucucacuauuuucccucaccaauggaucaaccugaggaccaacaacagugcaacgcuagugaugccuuacauaaacagcguccccauggauaacauguucaggcacaauaacuuaacacuaaugauuaucccauuuguaccgcuuaauuacagcgagggauccucaccguacgugcccauaacagucaccaucgcuccuaugugugcagaguacaacgggcuacggcuggccagcaaccagggucuaccaguuaugacaacaccugggagcacgcaauuucuaacuucagaugacuuccagucaccgucagcgaugccacaguuugaugucaccccagagaugcaaauaccaggccgcguaaauaauuugauggagauugccgagguggacucaguggugccugugaauaacacagaggacaacgugaguagccuuaaggcauaccagauuccaguacaguccaacagugacaaugguaagcagguuuuugguuuucccuuacaaccgggugccaacaacguucugaauagaacccucuugggugaaauucuaaacuauuacacccauuggaguggcagcauaaaacuuacauucauguucugugggucugccauggccacuggcaaguuccuucuggcauacucaccaccuggagcaggagucccgaaaaaccgaaaagaugcuaugcugggcacccacgucauaugggauguuggguuacaaucaagcugcguguugugcguaccguggauuagccaaacacacuacagauacgugguugaggaugaauacacagcagccggauauguuacaugcugguaucaaacaaauaucguggugccagcugaugugcaaagcucaugugacaucuugugcuuuguuucagccugcaacgauuucucugugcggaugcugaaagauacgcccuuuauaagacaggacacauuuuaucacggcccaguggaggagucuguggaucgugcaguggcacguguggcggacacgauuaguucacggcccacuaacucggagucaauuccagcgcucaccgccgccgagacugggcauaccucccaaguugugccuagcgacaccaugcaaacaagacaugugaaaaacuaccacucacgguccgaaucguccauugaaaauuuccuaugccggucugccuguguauacuaugccacauacaccaauaacucaaaaaaagagggguuugcagaguggguuaucaacacuagacagguggcacagcuaagaagaaaguuggagcucuucacauaucuaagguuugacuuagaacuaacauuuguuauaacgagcgcacaacaacccaguaccgccuccaguguagacgcucccgugcaaacccaucagauuauguaugugccuccagguggcccuguaccaacaaagguuaaagauuaugcauggcaaacuuccacaaaccccagcguuuucuggacagaaggaaacgccccaccaaggauguccaucccauucauuagcauugguaaugcguauaguuguuuuuaugauggguggacgcaguuuucaagaaacggggucuauggcaucaauacccucaacaacaugggcacguuguauaugaggcaugucaaugaagcggggcaggguccaaucaaaaguacuguuagaauauauuucaaacccaagcacgugaaggcgugggugccacguccgccaagauugugucaauaugagaagcagaaaaaugucaauuuuagcccuauaggaguaaccacaaguagaacggacauuauaacaacaggcacuuuuggucagcaguggcugcagagccugcugcucuugggcacuguggccugcagcaucucugcacccgcccgcucgcccagccccagcacgcagcccugggagcaugugaaugccauccaggaggcccggcgucuccugaaccugaguagagacacugcugcugagaugaaugaaacaguagaagucaucucagaaauguuugaccuccaggagccgaccugccuacagacccgccuggagcuguacaagcagggccugcggggcagccucaccaagcucaagggccccuugaccaugauggccagccacuacaagcagcacugcccuccaaccccggaaacuuccugugcaacccagauuaucaccuuugaaaguuucaaagagaaccugaaggacuuucugcuugucauccccuuugacugcugggagccaguccaggagaccacaaguagaacggacauuauaacaacaggcacuuuuggucagcaguccggagcgauauaugugggcaauuacaggauagucaauaggcaccuggcaacacacuuugacuggcaaaauugcauaugggaggauuacaacagggaccuccuaguaugcacuacaacggcgcacgguugugacaauauugcaagaugccagugcacagcagguguguauuauugugcuucuaggaacaagcauuacccggugcauuuugaaggaccaggcuugguggagguccaggaaagugaauauuauccaaaaagguaucaaucacacgugcuucuugccgcaggguuuucugagccgggggauuguggugguauucucaggugugaacaugguguuauaggcaucgugaccauggguggugaggguguugucggcuuugccgaugugcgcgaucucuuauggcuggaagacgacgcaauggaacagggagucagggacuacguggaacagcuuggaaacgccuuugguucuggguuuaccaaccagauuugcgagcaagucaaucuguuaaaagagucacuaauaggccaagacuccaucuuggaaaaaucacucaaggcauuagugaaaaucauaucagcacugguuaucguggugagaaaccacgaugaccucauaacagucacugccaccuuagcccugauuggcuguaccacuucaccguggcgguggcuaaagaaaaaagugacucaauacuaugggauucccauggccgagagacagaacaauggcuggcucaagaaauucacagaaaugaccaaugcaugcaagggcauggaauggaucgccaucaaaauccaaaaguucauugaguggcucaaaauuaagaucuugccagaaguaaaggaaaaacacgaguuucuuaccagacuuaagcaacucccacucuuagaaagccaaauugccaccaucgagcagagugcaccaucacagagugaucaggaacaacuguucuccaauauacaguacuucgcgcacuauugcagaaaguacgccccacuguacgcagcugaggcaaaaagaguguucucucuagaaaagaaaaugagcaacuacauacaguucaaguccaaaugccguauugaaccagucuguuuauugcuccaugguagcccuggagccggaaaauccguggcuaccaauuugauuggacgcucacuugcagagaagcucaacaguucaguguauucauugccaccagacccagaccauuuugacggcuauaaacaacaagcugucguaaucauggaugaccugugccagaacccagaugggaaagacguguccuuguucugccagaugguuucaaguguagacuuuguuccaccaauggcagcacuugaggagaaaggcauacucuucacaucgccuuucgugcucgccucuaccaacgcagguuccaucaacgcucccacugugucagacagcagagcccuugcuagaagguuccacuucgacuugaacauugaggucaucucaauguacagccagaauggcaaaaucaacaugccaaugucagugaaggcuugugaugaugaguguuguccaguuaauuucaagagguguuguccauuagugugcgguaaggccauucaguucauugacagaagaacacaaaugagguauucacuagacaugcuuguaacugaaauguucagggaguacaaccacagacauagugugggggcaacacucgaggcacucuuccaaggcccacccguguacaaagagaucaagaucaguguugcgccagaaaccccucccccaccggcaauagcagaucugcugaaaucuguggacagugaagcaguuagagaguacugcaaggaaaagggaugguuggugccugagaucaauuccacucuacaaauugaaaagcacgugagcagggccuucauuugccugcaagcgcucaccacguuuguuuccguggcggggauaauuuacaucauauacaaauuguuugcuggcuuccagggggccuacaccgguaugccaaaccaaaaaccuaaaguaccaacuuugaggcaggcuaaggugcagggaccggcauucgaauucgcuguggcaaugaugaaaagaaaugcuagcacagugaagaccgaguacggugaguucacaaugcuuggaaucuaugacaggugggcagugcuaccucgucacgcuagaccagggccuaccauccuaaugaaugaccaggaagugggugugguggaugccaaggaguuaguggacaaggauggcaccaauuuagaauuaacacucuugaagcucaauagaaaugagaaauucagagacauccgaggcuucuuaacacgugaggaggccgaagucaacgaggcugugcuugcuauuaacaccagcaaguucccaaauauguacauuccaguugggcaaguaacagauuauggcuuccucaacuuggguggcacaccaacuaagcgcaugcugauguacaacuucccaacacgugcaggccaaugugggggaguucuaaugucuacagguaaagugcucggaauacacgucggugguaauggacaccaaggguuuucggcagcccuguuacgccacuauuucaaugacgagcagggagagauugaguucaucgagagcucuaaggaugcaggcuuuccagucauuaacacaccuagcaaaacaaagcucgagcccaguguuuuccaucacguguuugagggaaacaaagaaccagcaguguugaggaauggagacccaagacugaaagccaauuuugaggaagccauuuucuccaaguacaucgggaaugugaacacacauguagacgaguacaugauggaagccgucgaccauuaugcaggacaguuagcuacuuuagauaucaguacagaaccuaugaagcuugaggaugcugucuacgguacugagggcuuggaagcucuggacuugaccacuagugcuggguaucccuaugucgcccuugguauaaaaaagagagacaucuuauccaaaaagucuaaggaccugucuaaacugagagaguguauggacaaguauggguuaaaccucccuauggucaccuaugugaaggaugaauugaggucagcagagaaaguggcuaagggcaaauccagacuaaucgaggcguccaguuuaaaugacucaguggcaaugagacagacuuucgguaaccuguauaagacauuccaucugaacccgggcauagugacuggcagugcaguuggaugcgaucccgacuuguucuggagcaagauuccagucaugcucgauggucaccucaucgccuuugacuacaguggauaugaugccagccugaguccggugugguuugcuugcuugaaacuguuguuggaaaagcugggguacucacacaaggaaacuaauuacauagauuacuugugcaacucccaccauuuguacagagacaagcacuacuuugugaggggaggaaugccauccggguguucuggaaccagcauuuucaauucaaugauuaauaacauaaucaucagaacacugauguugaagguguacaaagggauagaucuagaccaauuuaggaugauugcauauggugacgacgucauugcuucguacccguauccaauugaugcgucauugcuggcugaggcuggcaagggguauggucugauuaugacaccagcagacaaaggagaguguuucaaugaaguaacguggaccaacgucacuuuccugaaaagauacuucagagcggaugagcaauaucccuuccucguucacccagugaugccaaugaaggacauucaugagucaauuagguggacuaaagaccccaaaaacacccaagaucacgugcgauccuugugcuuauuggccuggcacaauggagagcaagaauaugagaauuuuguauccaaaaucagaagcgucccaguugggcgcugcuugacauugccugcguucucgacucugcgcaggaagugguuggauuccuucuaaaauugaaguacaaugugauuuaacucaauuggcuuaacccuaccacacuuaccgaacuagauaacggugugguagggguaaauuc

SEQ ID NO: 16 - RNA, 8214 nt, Artificial Sequence
  Genomic sequence of CVB1-Anti-PD1
uccuguggguugaucccacccacagggcccacugggcgcuagcacacugguauuccgguaccuuugugcgccuguuuuauacacccuucccaaguguaacuuagaagcuuaucacacacggucaacaggugacucaguaugccaacugggucuugaucaagcacuucuguuuccccggacugaguaucaauaagcugcucacgcggcugaaggagaaaacguucguuaaccggccaauuacuucgagaaaccuaguaacaccauggagguugcgcaguguuucgcuccacacaaccccaguguagaucaggccgaugagucaccgcacuccucacgggcgaccguggcgguggcugcgcuggcggccugcccaugggauaacccaugggacgcuucaauacugacauggugcgaagagucuauugagcuaauugguaguccuccggccccugaaugcggcuaauccuaacugcggagcagauacccacacgccagugggcagucugucguaaugggcaacucugcagcggaaccgacuacuuuggguguccguguuuccuuuuauuuuuauacuggcugcuuauggugacaauugagagauuguuaccauauagcuauuggauuggccauccagugacaaacagagcgauuauuuauuuauuuguugguuauaucccauuaagccaaaaggcccuaguuacucucaacuuuauacuauugcuuaauacaacgaaaugggagcucaggugucuacacaaaagacgggugcacaugagacuggcuugaaugccagcggcaauucugucauccauuacaccaacaucaauuacuacaaggaugcugcauccaacuccgcaaauaggcaagacuucacacaagacccagguaaguucacugaaccuguaaaggacaucauggugaaaacuaugccugcguugaacucacccucugcugaggaaugcggcuacagugacagagugcguuccaucacauuagguaauuccacuaucaccacccaagagugugccaacguggucgucggguacggaguguggccagaguaucuaaaagacaacgaggccacagccgaagaucagcccacacaaccugauguggccaccugccgcuucuauacuuuagaaucagugcaguggaugaaaaauucggccggguggugguggaagcugccugaugcacugucacagaugggcuuguuuggucaaaacaugcaguaccacuacuugggaagaacaggguauacuauacacguacaaugcaaugccucaaaguuccaucaaggaugccugcuuguagugugugugccugaagcugagauggggugcucuaacuugaacaacacgcccgaauucagugaguuauccggaggagacagugccagaauguucaccgauacacaggucggggagucaaacgccaagaaaguacagacagcgguguggaacgccggaaugggcguuggaguggguaaucucacuauuuucccucaccaauggaucaaccugaggaccaacaacagugcaacgcuagugaugccuuacauaaacagcguccccauggauaacauguucaggcacaauaacuuaacacuaaugauuaucccauuuguaccgcuuaauuacagcgagggauccucaccguacgugcccauaacagucaccaucgcuccuaugugugcagaguacaacgggcuacggcuggccagcaaccagggucuaccaguuaugacaacaccugggagcacgcaauuucuaacuucagaugacuuccagucaccgucagcgaugccacaguuugaugucaccccagagaugcaaauaccaggccgcguaaauaauuugauggagauugccgagguggacucaguggugccugugaauaacacagaggacaacgugaguagccuuaaggcauaccagauuccaguacaguccaacagugacaaugguaagcagguuuuugguuuucccuuacaaccgggugccaacaacguucugaauagaacccucuugggugaaauucuaaacuauuacacccauuggaguggcagcauaaaacuuacauucauguucugugggucugccauggccacuggcaaguuccuucuggcauacucaccaccuggagcaggagucccgaaaaaccgaaaagaugcuaugcugggcacccacgucauaugggauguuggguuacaaucaagcugcguguugugcguaccguggauuagccaaacacacuacagauacgugguugaggaugaauacacagcagccggauauguuacaugcugguaucaaacaaauaucguggugccagcugaugugcaaagcucaugugacaucuugugcuuuguuucagccugcaacgauuucucugugcggaugcugaaagauacgcccuuuauaagacaggacacauuuuaucacggcccaguggaggagucuguggaucgugcaguggcacguguggcggacacgauuaguucacggcccacuaacucggagucaauuccagcgcucaccgccgccgagacugggcauaccucccaaguugugccuagcgacaccaugcaaacaagacaugugaaaaacuaccacucacgguccgaaucguccauugaaaauuuccuaugccggucugccuguguauacuaugccacauacaccaauaacucaaaaaaagagggguuugcagaguggguuaucaacacuagacagguggcacagcuaagaagaaaguuggagcucuucacauaucuaagguuugacuuagaacuaacauuuguuauaacgagcgcacaacaacccaguaccgccuccaguguagacgcucccgugcaaacccaucagauuauguaugugccuccagguggcccuguaccaacaaagguuaaagauuaugcauggcaaacuuccacaaaccccagcguuuucuggacagaaggaaacgccccaccaaggauguccaucccauucauuagcauugguaaugcguauaguuguuuuuaugauggguggacgcaguuuucaagaaacggggucuauggcaucaauacccucaacaacaugggcacguuguauaugaggcaugucaaugaagcggggcaggguccaaucaaaaguacuguuagaauauauuucaaacccaagcacgugaaggcgugggugccacguccgccaagauugugucaauaugagaagcagaaaaaugucaauuuuagcccuauaggaguaaccacaaguagaacggacauuauaacaacaggcacuuuuggucagcagaugaagcaccugugguucuuccugcugcugguggccgcuccuaggugggugcugucccaggugcagcuggugcagagcggcguggaggugaagaagcccggcgcuuccgugaagguguccugcaaggccuccggcuacaccuucaccaacuacuacauguacugggugaggcaggccccuggacagggacuggaguggaugggcggcaucaacccuuccaacggcggcaccaacuucaacgagaaguucaagaaccgggugacccugaccaccgacuccuccaccaccaccgccuacauggagcugaagucccugcaguuugacgacaccgccguguacuacugcgccaggagggacuaccgguucgacaugggcuucgacuacuggggccagggcacaaccgugaccguguccagcggagguggcggaucuggagggggugguagcgguggaggcgggagugagaucgugcugacccaguccccugcuacacugucccugucccccggcgagagggcuacacugagcugcagggccuccaagggcguguccaccuccggcuacuccuaccugcacugguaccagcagaagccuggacaggcucccaggcugcugaucuaccuggccuccuaccuggaguccggcgugccugcuagguuuuccggcagcggcagcggcaccgauuucacccugaccaucuccucccuggagcccgaggacuucgccguguacuacugccagcacuccagggaucugccucugaccuucggcggcggcaccaagguggagaucaagaccacaaguagaacggacauuauaacaacaggcacuuuuggucagcaguccggagcgauauaugugggcaauuacaggauagucaauaggcaccuggcaacacacuuugacuggcaaaauugcauaugggaggauuacaacagggaccuccuaguaugcacuacaacggcgcacgguugugacaauauugcaagaugccagugcacagcagguguguauuauugugcuucuaggaacaagcauuacccggugcauuuugaaggaccaggcuugguggagguccaggaaagugaauauuauccaaaaagguaucaaucacacgugcuucuugccgcaggguuuucugagccgggggauuguggugguauucucaggugugaacaugguguuauaggcaucgugaccauggguggugaggguguugucggcuuugccgaugugcgcgaucucuuauggcuggaagacgacgcaauggaacagggagucagggacuacguggaacagcuuggaaacgccuuugguucuggguuuaccaaccagauuugcgagcaagucaaucuguuaaaagagucacuaauaggccaagacuccaucuuggaaaaaucacucaaggcauuagugaaaaucauaucagcacugguuaucguggugagaaaccacgaugaccucauaacagucacugccaccuuagcccugauuggcuguaccacuucaccguggcgguggcuaaagaaaaaagugacucaauacuaugggauucccauggccgagagacagaacaauggcuggcucaagaaauucacagaaaugaccaaugcaugcaagggcauggaauggaucgccaucaaaauccaaaaguucauugaguggcucaaaauuaagaucuugccagaaguaaaggaaaaacacgaguuucuuaccagacuuaagcaacucccacucuuagaaagccaaauugccaccaucgagcagagugcaccaucacagagugaucaggaacaacuguucuccaauauacaguacuucgcgcacuauugcagaaaguacgccccacuguacgcagcugaggcaaaaagaguguucucucuagaaaagaaaaugagcaacuacauacaguucaaguccaaaugccguauugaaccagucuguuuauugcuccaugguagcccuggagccggaaaauccguggcuaccaauuugauuggacgcucacuugcagagaagcucaacaguucaguguauucauugccaccagacccagaccauuuugacggcuauaaacaacaagcugucguaaucauggaugaccugugccagaacccagaugggaaagacguguccuuguucugccagaugguuucaaguguagacuuuguuccaccaauggcagcacuugaggagaaaggcauacucuucacaucgccuuucgugcucgccucuaccaacgcagguuccaucaacgcucccacugugucagacagcagagcccuugcuagaagguuccacuucgacuugaacauugaggucaucucaauguacagccagaauggcaaaaucaacaugccaaugucagugaaggcuugugaugaugaguguuguccaguuaauuucaagagguguuguccauuagugugcgguaaggccauucaguucauugacagaagaacacaaaugagguauucacuagacaugcuuguaacugaaauguucagggaguacaaccacagacauagugugggggcaacacucgaggcacucuuccaaggcccacccguguacaaagagaucaagaucaguguugcgccagaaaccccucccccaccggcaauagcagaucugcugaaaucuguggacagugaagcaguuagagaguacugcaaggaaaagggaugguuggugccugagaucaauuccacucuacaaauugaaaagcacgugagcagggccuucauuugccugcaagcgcucaccacguuuguuuccguggcggggauaauuuacaucauauacaaauuguuugcuggcuuccagggggccuacaccgguaugccaaaccaaaaaccuaaaguaccaacuuugaggcaggcuaaggugcagggaccggcauucgaauucgcuguggcaaugaugaaaagaaaugcuagcacagugaagaccgaguacggugaguucacaaugcuuggaaucuaugacaggugggcagugcuaccucgucacgcuagaccagggccuaccauccuaaugaaugaccaggaagugggugugguggaugccaaggaguuaguggacaaggauggcaccaauuuagaauuaacacucuugaagcucaauagaaaugagaaauucagagacauccgaggcuucuuaacacgugaggaggccgaagucaacgaggcugugcuugcuauuaacaccagcaaguucccaaauauguacauuccaguugggcaaguaacagauuauggcuuccucaacuuggguggcacaccaacuaagcgcaugcugauguacaacuucccaacacgugcaggccaaugugggggaguucuaaugucuacagguaaagugcucggaauacacgucggugguaauggacaccaaggguuuucggcagcccuguuacgccacuauuucaaugacgagcagggagagauugaguucaucgagagcucuaaggaugcaggcuuuccagucauuaacacaccuagcaaaacaaagcucgagcccaguguuuuccaucacguguuugagggaaacaaagaaccagcaguguugaggaauggagacccaagacugaaagccaauuuugaggaagccauuuucuccaaguacaucgggaaugugaacacacauguagacgaguacaugauggaagccgucgaccauuaugcaggacaguuagcuacuuuagauaucaguacagaaccuaugaagcuugaggaugcugucuacgguacugagggcuuggaagcucuggacuugaccacuagugcuggguaucccuaugucgcccuugguauaaaaaagagagacaucuuauccaaaaagucuaaggaccugucuaaacugagagaguguauggacaaguauggguuaaaccucccuauggucaccuaugugaaggaugaauugaggucagcagagaaaguggcuaagggcaaauccagacuaaucgaggcguccaguuuaaaugacucaguggcaaugagacagacuuucgguaaccuguauaagacauuccaucugaacccgggcauagugacuggcagugcaguuggaugcgaucccgacuuguucuggagcaagauuccagucaugcucgauggucaccucaucgccuuugacuacaguggauaugaugccagccugaguccggugugguuugcuugcuugaaacuguuguuggaaaagcugggguacucacacaaggaaacuaauuacauagauuacuugugcaacucccaccauuuguacagagacaagcacuacuuugugaggggaggaaugccauccggguguucuggaaccagcauuuucaauucaaugauuaauaacauaaucaucagaacacugauguugaagguguacaaagggauagaucuagaccaauuuaggaugauugcauauggugacgacgucauugcuucguacccguauccaauugaugcgucauugcuggcugaggcuggcaagggguauggucugauuaugacaccagcagacaaaggagaguguuucaaugaaguaacguggaccaacgucacuuuccugaaaagauacuucagagcggaugagcaauaucccuuccucguucacccagugaugccaaugaaggacauucaugagucaauuagguggacuaaagaccccaaaaacacccaagaucacgugcgauccuugugcuuauuggccuggcacaauggagagcaagaauaugagaauuuuguauccaaaaucagaagcgucccaguugggcgcugcuugacauugccugcguucucgacucugcgcaggaagugguuggauuccuucuaaaauugaaguacaaugugauuuaacucaauuggcuuaacccuaccacacuuaccgaacuagauaacggugugguagggguaaauuc

SEQ ID NO: 17 - DNA, 22 bp, Artificial Sequence
  DNA sequence of miR-133 target sequence
acagctggttgaaggggaccaa

SEQ ID NO: 18 - DNA, 22 bp, Artificial Sequence
  DNA sequence of miR-206 target sequence
ccacacacttccttacattcca

SEQ ID NO: 19 - DNA, 102 bp, Artificial Sequence
  DNA sequence of tandem sequence of miR-133 target sequence and miR-206 target sequence
acagctggttgaaggggaccaacgatacagctggttgaaggggaccaaaccggtccacacacttccttacattccatcacccacacacttccttacattcca

SEQ ID NO: 20 - DNA, 507 bp, Artificial Sequence
  DNA sequence of internal ribosome entry site sequence of HRV2
aacttagaagtttttcacaaagaccaatagccggtaatcagccagattactgaaggtcaagcacttctgtttccccggtcaatgttgatatgctccaacagggcaaaaacaactgcgatcgttaaccgcaaagcgcctacgcaaagcttagtagcatctttgaaatcgtttggctggtcgatccgccatttcccctggtagacctggcagatgaggctagaaataccccactggcgacagtgttctagcctgcgtggctgcctgcacaccctatgggtgtgaagccaaacaatggacaaggtgtgaagagccccgtgtgctcgctttgagtcctccggcccctgaatgtggctaaccttaaccctgcagctagagcacgtaacccaatgtgtatctagtcgtaatgagcaattgcgggatgggaccaactactttgggtgtccgtgtttcactttttcctttatatttgcttatggtgacaatatatacaatatatatattggcacc